(19) United States
(12) Patent Application Publication
(10) Pub. No.: US 2024/0263002 A1
(43) Pub. Date: Aug. 8, 2024

(54) ELECTROCONDUCTIVE HYDROGEL AND DEVICES WITH CONDUCTING POLYMERS ASSEMBLED AROUND A 3D NANOFIBER FRAMEWORK

(71) Applicant: The University of Hong Kong, Hong Kong (CN)

(72) Inventors: Lizhi XU, Changsha (CN); Huimin HE, Kaihua (CN); Hao LI, Zunhua (CN)

(73) Assignee: The University of Hong Kong, Hong Kong (CN)

(21) Appl. No.: 18/431,202

(22) Filed: Feb. 2, 2024

Related U.S. Application Data
(60) Provisional application No. 63/443,628, filed on Feb. 6, 2023.

(51) Int. Cl. C08L 29/04; A61B 5/251; A61B 5/268; A61B 5/28; A61B 5/296; A61L 31/04; A61L 31/14; C08J 3/075; F24S 70/14

(52) U.S. Cl. CPC C08L 29/04; A61B 5/251; A61B 5/268; A61B 5/28; A61B 5/296; A61L 31/041; A61L 31/145; C08J 3/075; F24S 70/14; A61B 2562/0209; A61L 2400/12; C08J 2329/04; C08J 2439/04; C08J 2477/00; C08L 2203/02; C08L 2205/03; C08L 2205/16

(57) ABSTRACT

An electroconductive hydrogel is formed by hybrid assembly of polymeric nanofiber networks of conducting polymers that self-organize into highly connected 3D nanostructures with an ultralow threshold (˜1 wt %) for electrical percolation. A method for forming the electroconductive hydrogel comprises the steps of: dispersing aramid nanofibers (ANFs) in dimethyl sulfoxide (DMSO); conducting a solvent exchange with water to generate hydrogels with connective 3D fibrillar networks that serve as templates for the assembly of conducting polymers; incorporating polyvinyl alcohol (PVA) during the processing of the hydrogels to weld the fibrillar joints via hydrogen bonding; infiltrating monomers into the nano-porous hydrogels in an aqueous media; and polymerizing the hydrogels with added oxidants.

### CROSS-REFERENCE TO RELATED PATENT APPLICATIONS

This application claims the benefit of priority under 35 U.S.C. Section 119(e) of U.S. Application No. 63/443,628, filed Feb. 6, 2023, which is incorporated herein by reference in its entirety.

### TECHNICAL FIELD

The present invention relates to electroconductive hydrogels and, more specifically, to electroconductive hydrogels based on a hybrid assembly of polymeric nanofiber networks.

### BACKGROUND

Electroconductive hydrogels exhibit great potential for applications in implantable bioelectronics, tissue engineering platforms, soft actuators, and other emerging technologies. In particular, hydrogels are promising candidate materials for the construction of soft electronics and biomedical devices due to their mechanical flexibility, structural permeability and biocompatibility.

However, achieving high conductivity and mechanical robustness in hydrogels remains challenging. Most traditional hydrogels are electrically inactive. However, recent research has begun to explore conductive hydrogels, creating new possibilities for implantable bioelectrodes [1,2], soft actuators [3,4], tissue engineering platforms [5,6], solar-powered water treatment [7,8], and other advanced technologies [9-12]. Nevertheless, despite extensive research efforts, achieving high electrical conductivity and mechanical robustness in hydrogels still remains challenging, which limits their practical applications [13]. For instance, hydrogels loaded with mobile ions usually exhibit a conductivity at the level of ˜0.1 S/cm [14]. This value is several orders of magnitude lower than those of electronic conductors, partly because of the low mobility of ionic charge carriers [15,16]. Incorporating electronic conductors such as metal nanowires [5,17], carbon nanotubes [18,19], or conducting polymers [11,20] into hydrogels does not result in a high conductivity of the hybrid composites that is comparable to those of the fillers. This discrepancy is due to the fact that the conductive fillers are randomly distributed in the hydrogel matrix, and it is difficult to achieve electrical percolation with low volume fraction (e.g., <10%) of the conductive phase [21-23]. On the other hand, increasing the concentration of the conductive fillers may compromise the mechanical properties, water content, or other attributes of the hydrogels which are essential for their functional applications.

Recently, organizing conducting polymers into a nanoscale network was exploited for imparting high electrical conductivity to hydrogels. For example, porous structures of polyaniline (PANi) induced by freezing have led to a significant increase of conductivity for hydrogels as compared with those with randomly distributed PANI particles [24]. In another scheme, aggregation of poly(3,4-ethylenedioxythiophene):polystyrene sulfonate (PEDOT:PSS) by solvent treatment and/or dry annealing resulted in hydrogels with excellent conductivity on the order of 10-47 S/cm [1,25,26]. However, these hydrogels usually exhibit low mechanical strengths (e.g., ˜0.2 MPa) partly due to the intrinsic brittleness of the constituent conducting polymers. It is difficult to incorporate toughening components into these hydrogels without interfering with the electroconductive networks [27]. Furthermore, the dramatic volume change or high temperature annealing involved in the materials processing raises challenges for their patterning and fabrication into hybrid devices.

U.S. Pat. No. 11,111,343 B2 describes pure aramid nanofibers (ANFs) without electronic functions and without compositions and processing methods. Thus, they lack high conductivity and mechanical robustness. An article by Yuk et al., “Hydrogel Bioelectronics,” Chem. Soc. Rev., 2019, 48, 1642 (2019) reviews the state-of-the-art of hydrogels in bioelectronic applications including those with electroconductivity. However, none of them achieved high conductivity, mechanical robustness, and manufacturability.

Flexible devices have drawn gaining attention due to their critical role in applications for energy harvesting and storage [37,38], human-machine interactions [39,40], and personalized healthcare[41,42] in modern society. For instance, stretchable supercapacitors have undergone rapid progress and are becoming a necessary element for energy storage and supply in wearable electronic devices due to their high-power density and long-term stability [43,44]. Soft bioelectronics in close contact with biological systems have been extensively explored for applications involving drug delivery, diagnosis, electrical modulation of tissues and organs, etc. [49-51]. To guarantee the reliable functioning of flexible devices in such applications, high-performance electrodes are required to possess superior electrical/electrochemical, mechanical, and stability properties simultaneously in one single materials system [52,53]. Traditional electrodes made from carbon and metal materials have high electrical conductivity, but their limited capacitive properties result in compromised electrode performance at low-frequency alternating currents [54]. Surface modification with nanoarchitectures provides a promising way to improve the capacitive performance of electrodes. For instance, conventional electrode materials, such as carbon, platinum, and titanium nitride, elaborated with nanostructured surfaces exhibit markedly enhanced capacitance and charge injection limits [55]. However, nano-structuring processes may interfere with the structural integrity and create ‘dead’ volumes, leading to the deterioration of electrode performance and the emergence of other complications [44].

Electrically conductive hydrogels are highly hydrated materials with inherent porosity, simultaneously allowing efficient mass and charge transfer within the materials. Recently, electrodes modified from conducting polymer hydrogels (i.e., nonconductive hydrogel matrices incorporated with conducting polymers) have been extensively explored for device applications due to their favorable electrochemical and mechanical properties, stability, and biocompatibility. However, conducting polymer hydrogels produced by simply mixing or polymerizing conducting polymers within hydrogel matrices typically show compromised electrical conductivity below 0.2 S cm⁻¹, as well as high electrical percolation threshold, due to arbitrary and low-connectivity conductive pathways built by conducting polymers [54]. Notably, nano-structuring conducting polymer into nanoscale networks allows conductive hydrogels with excellent electrical properties, as exemplified by the bi-continuous nanonetworks (11 S cm⁻¹) of poly(3,4-ethylenedioxythiophene):polystyrene sulfonate (PEDOT:PSS) and hydrophilic polyurethane prepared by phase separation [49]. Although PEDOT:PSS-based conductive hydrogels have achieved significant progress on their electrical conductivity, capacitance, and mechanical properties, efforts are still needed to make their manufacturing process more adaptable and controllable, and ensure the polymer electrodes electrochemically more stable under long-term operation at extreme conditions (e.g., repeated swelling or saline solution).

However, flexible devices, such as stretchable supercapacitors and soft bioelectronics, have their practical performance limited by electrodes which are desired to be highly conductive, mechanically flexible and strong, porous, electrochemically stable, and biocompatible.

Solar evaporation is a promising approach to desalination due to its simple system configuration and high energy efficiency. [76,77] Extensive research efforts were recently invested into novel materials and structures for enhanced performance in solar desalination. [78-84] Among the variety of designs, hydrogels attracted wide attention due to their 3D hydrophilic networks that facilitate water replenishment and accelerate vapor generation via interactions with water molecules. [85-88] In particular, hydrogel-based solar evaporators (HSEs) have emerged as energy-efficient designs for water purification due to the reduced vaporization enthalpy in the hydrated polymeric network. However, most of the reported hydrogel-based solar evaporators (HSEs) can stably operate only with low salinity liquid (<10%), which does not satisfy the requirements for industrial application or prolonged use. [89,90]. It remains challenging for HSEs to achieve stable performance in desalination, partly due to the tradeoff between desired evaporation dynamics and salt tolerance.

Salt accumulation in HSEs often occurs during continuous evaporation especially in strong brine, which compromises the photothermal evaporation process by blocking water transport and solar reception. [91-94] Existing strategies for enhancing salt tolerance in HSEs include local crystallization, [93,95] ion rejection, [92,96] Janus design, [94,97] and back diffusion. [98-100] Among these approaches, promoting back diffusion of ions into the bulk water is advantageous since it aims to eliminate salt accumulation in HSEs with feasible structures. Engineered channels with low diffusive tortuosity were used for enhancing the required mass transfer. For instance, vertically aligned vessels were constructed in HSEs to accelerate back diffusion of ions via directional confinement of the water path. [101,102] However, heat loss to the bulk water is also aggravated with the enhancement of water flux, which compromises evaporation efficiency. Vertical radiant structures were developed to address the above tradeoff, [103] but the complicated fabrication renders it suboptimal for practical implementation. Alternative approaches aim to build open porous structures by porosigen, [104,105] gas-blowing, [106,107] and foaming polymerization, [108,109] which also create highly continuous water channels. Nevertheless, sophisticated preparation process and relatively low porosity of these fabricated porous hydrogels may limit their practical utilization.

Indeed, achieving optimum photothermal evaporation requires engineering of various physical processes that are mutually dependent. Although considerable efforts have been devoted to decoupling the water transfer and heat conduction in HSEs by regulating pore structures, the associated variation of vaporization enthalpy is usually overlooked, which actually affect the evaporation performance of HSEs. [100,110] Generally, the boosted water transfer by increasing porosity or pore diameter also results in an increase of vaporization enthalpy due to the weakened interaction between hydrophilic functional groups and water molecules, which is adverse to solar evaporation. [104] A labyrinth of nexus between microstructures, thermodynamics, and mass/heat transfer create difficulties for the optimization of HSEs. Careful designs regarding the complex interplay between physical processes are essential for achieving desired performance in solar evaporation.

Three-dimensional evaporators represent an energy efficient design for solar desalination to address the global water crisis. However, the lack of scalable fabrication strategies and inability to adapt the dynamic sunlight hinder their practical applications. Desalination via interfacial solar evaporators (ISEs) is regarded as a cost-effective and carbon-zero approach to the worldwide clean water shortage. [118,119] Lots of efforts have been devoted to design ISEs with enhanced evaporation efficiency. Recent advances include regulating water supply, [120,121] minimizing heat loss, [122,123] reducing vaporization enthalpy, [124,125] capturing ambient energy, [126,127] and so on. [128-130] Three-dimensional (3D) ISEs provides a versatile platform for incorporating diverse materials and macroscopic architectures to enhance evaporation efficiency by increasing evaporating surface and harvesting ambient energy. [131] Another benefit of 3D ISEs is their capability to perform localized crystallization by architectural designs, which facilitates solar desalination in high-salinity brine. [132-134] However, existing fabrications involve 3D printing techniques and complicated structures, hindering their practical applications due to the low productivity. Additionally, though efficient evaporation has been achieved in laboratory settings, the evaluations are conducted under ideal static solar illumination. The static nature of conventional ISEs misaligns with the dynamic solar trajectory, leading to the decreased evaporation efficiency in real-world utilizations. [135] Several works have begun to integrate ISEs with solar tracking platforms that usually used in photovoltaics. [136.137] Nevertheless, high installation cost and low compatibility to the existing evaporation system make it contrary to the original intention of ISEs. Exploring strategies for producing scalable and solar-trackable 3D ISEs are crucial for promoting their practical applications.

Kirigami designs endow static and rigid substrate with reconfigurability by rational cuts, [138] achieving various advances in soft robots, [139,140] wearable electronics, [141,142] energy harvesters [143] and so on. [144-146] The out-of-plane deformations by kirigami structure under tension yields various tunable 3D architectures, offering opportunities for engineering reconfigurable 3D ISEs. Indeed, kirigami-designs for solar trackable photovoltaics were explored to overcome underutilization of solar. [147,148] However, these designs are not feasible for solar evaporation system. On the one hand, the base material ought to be highly hydrophilic for efficient water transfer to the evaporating interface. Enough stiffness is also required to afford gravity of water during out-of-plane deformation. On the other hand, the kirigami-design must enable unilateral out-of-plane deformation for more evaporation area above water. Therefore, it is necessary to screen appropriate base materials and propose optimal structure design for kirigami-based evaporation system.

### SUMMARY OF THE INVENTION

The present invention involves a different material system for creating robust and electronically conductive hydrogels with desired manufacturability for device applications. This approach to forming electroconductive hydrogels is based on the hybrid assembly of polymeric nanofiber networks. In these hydrogels, conducting polymers self-organize into highly connected 3D nanostructures with an ultralow threshold (˜1 wt %) for electrical percolation, which is assisted by templating effects from aramid nanofibers (ANFs). In effect, this scheme uses the templating effect from a 3D hyper-connective network of nanofibers for guiding the assembly of conducting polymers. The resultant hybrid network of conductive nanofiber hydrogels (DNHs) exhibit a combination of high electronic conductivity and structural robustness without sacrificing porosity or water content.

A representative conductive nanofiber hydrogel (CNH) involving polypyrrole (PPy), ANFs and polyvinyl alcohol (PVA). The resultant CNHs exhibit a combination of high mechanical strength and electronic conductivity originating from the hybrid polymeric nanofiber network. In particular they achieve a combination of conductivity of ˜8,000 S m-1, mechanical strength of ˜9.4 MPa and stretchability of ˜36%, which is unachievable by other hydrogel materials.

Furthermore, the simple processing techniques for CNHs afford patterning of device arrays including electrodes and interconnects for enabling high-performance bioelectronic devices, showing favorable electrochemical impedance and charge injection capacity for electrophysiological applications on cells, tissues and organs. In addition, cardiomyocytes cultured on soft and conductive CNH substrates exhibit spontaneous and synchronous beating, suggesting opportunities for the development of advanced implantable devices and tissue engineering technologies. The outstanding electronic and mechanical properties of these CNHs, in conjunction with their manufacturability and biocompatibility, indicate new opportunities for the development of advanced soft bioelectronics and tissue engineering platforms.

An embodiment of the present invention provides an efficient one-step-synthesis method to create a conductive hydrogel satisfying all these necessary criteria for flexible high-performance electrochemical electrodes. Specifically, leveraging a hyper-connective nanofibrous network from aramid hydrogels as a template, as indicated above, the conducting polymer, polypyrrole, assembles conformally onto nanofibers through in-situ synthesis within the nanofibrous network, generating continuous nanostructured conductive pathways. As a result, the composite hydrogel has superior conductivity (72 S cm-1), fracture strength (27.2 MPa), and ductility (22.4%), as well as excellent electrical stability under extreme environments. Depending on theses merits, supercapacitors utilizing this hydrogel exhibit high specific capacitance (240 F g-1) and cyclic stability in acidic electrolytes. Furthermore, bioelectrodes of the patterned hydrogels provide favorable bioelectronic interfaces in physiological environments, allowing high-quality electrophysiological recording and stimulation. The new strategy to generate this all-polymer conductive hydrogel is readily scalable to electrodes for other flexible devices.

Composite hydrogels with tunable self-assembled nanofiber networks are exploited by another embodiment of the present invention for the engineering of solar evaporators with both high evaporation performance and resistance to salt accumulation. The nanofibrous hydrogel solar evaporators (NHSEs) present an intrinsic open network with high porosity, above 90%, enabling continuous water channels for efficient mass transfer. Theoretical modeling captures the complex nexus between microstructures and evaporation performance by coupling water transfer, thermal conduction, and vaporization enthalpy during evaporation. The mechanistic understanding and engineering tuning of the composites lead to an optimum configuration of NHSEs, which demonstrate a stable evaporation rate of 2.85 kg m-2 h-1 during continuous desalination in 20% brine. The outstanding performance of NHSEs and the underlying design principles facilitate further development of practical desalination systems.

As a still further embodiment, the present invention uses a Kirigami-engineered hydrogel membrane that enables scalable and solar-trackable 3D evaporator arrays. The robust hydrogel membrane incorporating hydrated polymeric network offers an ideal base material for kirigami evaporators. In this design, periodic triangular notches on the substrate provide reconfigurability that generates 3D conical arrays under uniaxial tension. An additional advantage of 3D conical arrays is their capability for localized crystallization for stable solar desalination, achieving an ultrahigh evaporation rate of 3.4 kg m-2 h-1 in saturated brine. Furthermore, the reconfigurable kirigami evaporator achieves dynamic solar tracking by coupling the tilt angle with solar trajectory, providing a readily available design for enhancing solar energy efficiency. The versatile kirigami based hydrogel evaporator may accelerate the implementation of solar evaporators and provide inspiration for other kirigami-enabled devices based on hydrogel.

### BRIEF SUMMARY OF THE DRAWINGS

This patent or application file contains at least one drawing executed in color. Copies of this patent or patent application publication with color drawing(s) will be provided by the Office upon request and payment of the necessary fee.

The foregoing and other objects and advantages of the present invention will become more apparent when considered in connection with the following detailed description and appended drawings in which like designations denote like elements in the various views, and wherein:

FIG. 1A is a schematic drawing of the synthesis of a percolated polypyrrole (PPy) network guided by the nanofiber template, FIG. 1B is an SEM image of PPy particles polymerized in aqueous solution without a nanofiber template, FIG. 1C is an SEM image with an aramid-polyvinyl alcohol (ANF-PVA) template, FIG. 1D is an SEM image with an aramid-polyvinyl alcohol-polypyrrole (ANF-PVA-PPy) network, FIG. 1E shows the electrical conductance of CNHs demonstrated by powering an array of LEDs and FIG. 1F shows photographs of serpentine CNH patterns integrated on ANF-PVA substrate before (left) and after (right) 80% elongation;

FIG. 2A shows graphs of the conductivity of conductive nanofiber hydrogels (CNHs) as a function of PPy content for pure ANF templates, FIG. 2B shows graphs of conductivity for ANF-PVA with constant solid fraction of the template, FIG. 2C shows graphs of conductivity for templates with a constant ratio between ANF and PVA, FIG. 2D shows that the electrical percolation threshold for PPy is dependent on the various configurations of the templates, FIG. 2E shows that high conductivity of CNHs is achieved as the PPy content is increased beyond the threshold of electrical percolation, FIG. 2F is a comparison of electrical conductivities between CNHs and other PPy-incorporated hydrogels prepared under similar polymerization conditions, showing the unique templating effects from ANFs;

FIG. 3A illustrates stress-strain curves of samples with various PPy content based on a pure ANF template, FIG. 3B shows stress-strain curves of samples based on an ANF-PVA template, FIG. 3C are bar graphs of the statistics of maximum elongation and conductivity for various CNHs with 1.9% ANF and 9.5% PVA, FIG. 3D is a graph of resistance changes as a function of tensile strain for CNHs with various PPy content, FIG. 3E is a schematic drawing illustrating the orientation of the nanofiber network in response to applied strain and FIG. 3F is an SEM image showing the orientation of the nanofiber network under stretching;

FIG. 4A is a plot of the capacitances of ANF-PPy normalized for area, FIG. 4B shows the capacitances normalized for volume, FIG. 4C is a graph of electrochemical impedance spectroscopy (EIS) of CNH and an Au electrode, FIG. 4D is a graph of electrochemical cyclic voltammetry (CV), FIG. 4E shows pulsed current injection curves for a CNH electrode (3.1% ANF, 15.5% PVA, 25% PPy) and an Au electrode with the voltage switching between −0.5 and 0.5 V, FIG. 4F shows a graph of cyclic electrochemical current pulse injection curves of electrodes based on ANF-PPY and an Au foil with a square wave, FIG. 4G is areal impedance of the ANF-PPy and an Au foil, FIG. 4H is a schematic illustration of the patterning process for CNH, FIG. 41 shows photographs of patterned CNH based on ANF-PVA films, FIG. 4J shows photographs of four-channel CNH electrodes and those electrodes mounted across a human forearm and FIG. 4K is a series of photographs of distinct hand actions and the corresponding EMG patterns recorded by CNH electrodes;

FIG. 5A shows CNHs interfaced with cardiomyocytes and immunofluorescent staining of cardiac-specific proteins, FIG. 5B shows imaging of calcium transients in cardiomyocytes cultured on TCP and temporal variations recorded at separate sites, showing distinct patterns of excitation, FIG. 5C shows imaging of calcium transients in cardiomyocytes cultured on CNH and temporal variations recorded at separate sites, showing distinct patterns of excitation, FIG. 5D shows cytocompatibility of hydrogels with live-dead staining of cardiomyocytes cultured on ANF and FIG. 5E shows staining on ANF-PPy hydrogels after 5 days;

FIG. 6A depicts graphs of Fourier-transform infrared (FTIR) spectra (I) of ANF, PVA, PPy, ANF-PPy, ANF-PVA, and ANF-PVA-PPy and a magnified plot (II) showing spectra of aramid C═O stretching vibration in ANF, ANF-PVA, ANF-PPy and ANF-PVA-PPy, and FIG. 6B shows graphs of Raman spectra of ANF-PVA, PPy, and ANF-PVA-PPy that indicate the incorporation of PPy in the composites;

FIG. 7A depicts C is XPS spectra of ANF-PVA and ANF-PVA-PPy and FIG. 7B is N is spectra of ANF-PVA and ANF-PVA-PPy;

FIG. 8A is a schematic of a circuit for measuring conductivity by the two-probe method, FIG. 8B is a graph of current measured with a bias voltage from −0.5 to 0.5 V by the two-probe method showing linear responses of CNHs, FIG. 8C is a schematic of a circuit for measuring conductivity by a four-point probe and FIG. 8D are bar graphs showing that the conductivities measured by the two different methods are consistent, based on various ANF-PVA-PPy samples with different levels of conductivity;

FIG. 9A provides photographs of a strip sample under tension (I) and hollow tubes which can withstand bending (II), FIG. 9B shows photographs of films with various shapes (I) and patterned by infrared laser machining (II), FIG. 9C shows photographs of ANF-PPy under substantial bending and FIG. 9D shows substantial twisting;

FIG. 10A shows stress-strain curves for 1.9% ANF-9.5% PVA and 3.1% ANF-15.5% PVA hydrogels, FIG. 10B is a graph showing that the PPy content in ANF-PVA-PPy hydrogels is tunable with various pyrrole concentrations during polymerization and FIG. 10C is a graph showing the water content of conductive hydrogels;

FIG. 11A is an illustration of slices of a bulk ANF-PVA-PPy hydrogel with 100 μm-thick layers, FIG. 11B is a graph of the PPy content of each layer as a function of the depth from the surface and FIG. 11C is a graph of the electrical conductivity as a function of PPy content for CNHs with 3.1% ANF-15.5% PVA matrix;

FIG. 12 illustrates the effects of PPy contents on the conductivity of ANF-PPy hydrogels;

FIG. 13 shows a comparation of conductivities between CNHs and other reported conductive hydrogels;

FIG. 14 is a schematic illustration of possible intermolecular interactions in ANF-PVA-PPy;

FIG. 15A shows SEM images of ANF-PPy in fibrillar networks while PPy forms as spherical particles randomly distributed in the PVA matrix and FIG. 15B shows SEM images based on cross-sections of hydrogel samples;

FIG. 16A is stress-strain curves showing the tensile behaviors of CNHs with various PPy contents based on matrices with 3.1% ANF-15.5% PVA and FIG. 16B is bar graphs of the maximum elongation and conductivity of various samples;

FIG. 17 shows the tensile stress-strain curve for a representative CNH;

FIG. 18A shows cyclic stress-strain curves for CNHs based on 1.9% ANF, 9.5% PVA, 10% PPy and FIG. 18B shows similar cyclic curves for CNHs based on 3.1% ANF, 15.5% PVA, 15% PPy;

FIG. 19A shows graphs of mechanical and electrical behaviors of CNHs under tensile deformation where the samples are based on 1.9% ANF 9.5%-PVA matrices incorporated with a PPy content of 25 wt % (I), 20 wt % (II), 15 wt % (III), 10 wt % (IV), and 5 wt % (V) and FIG. 19B is a bar graph comparison of electrical responses of CNHs and PVA-PPy on tensile deformation of 10%;

FIG. 20A shows a schematic of a setup for measurement of ECG using CNH electrodes and FIG. 20B shows ECG signals captured by the CNH electrodes, showing details of QRS, T and P waves;

FIG. 21A illustrates immunostaining of troponin (red) and nuclei (blue) of neonatal rat cardiomyocytes cultured on TCPs, ANF-PVA and ANF-PVA-PPy respectively, and FIG. 21B shows immunofluorescent staining of cardiomyocytes cultured on ANF-PPy after 5 days;

FIG. 22A shows photos of water droplets (3 μl) on the surfaces of ANF-PVA-PPy (3.1% ANF, 15.5% PVA) with various PPy contents after 10s contact showing the wettability of ANF-PVA-PPy with different PPy contents and a bar graph of water contact angles as a function of ANF and ANF-PPy, FIG. 22B is a bar graph of water contact angles as a function of PPy content, FIG. 22C is a bar graph showing a correlation between the solid content of ANF hydrogels and the conductivity of resulting ANF-PPy hydrogels and FIG. 22D shows the differences in the QCM frequency change as a function of time for ANF and ANF-PPy hydrogels after contact with fetal bovine serum (FBS);

FIG. 23A is a schematic illustrating the architecture of ANF-PPy, FIG. 23B shows SEM images of the nanostructures of PPy polymerized in water without templates (left), ANF nanofibrous network (middle), and ANF-PPy with PPy polymerized within ANF hydrogel network (right), FIG. 23C is an illustration of a thin ANF-PPy film of 100 m thickness loaded with 500 g weight, FIG. 23D is image of an electrical circuit with LEDs interconnected by laser-patterned ANF-PPy leads, FIG. 23E is an image of an ANF-PPy of Peano curve printed on a soft PDMS and FIG. 23F is a soft electronic circuit built with a serpentine ANF-PPy to link an LED light on a soft PDMS (2 cm×2 cm);

FIG. 24A shows the chemical composition characterized by FTIR spectroscopy of ANF, PPy, and ANF-PPy and FIG. 24B shows magnified plots showing spectra of aramid C═O stretching vibration;

FIG. 25A shows molecular interactions of ANF hydrogel in Py solution for binding free energy between ANF, H₂O, and Py molecules and FIG. 25B shows the distribution of H₂O and Py molecules in a localized area near ANF surface;

FIG. 26A shows noncovalent interactions between ANF and PPy as a reduced density gradient (RDG) function isosurface map of ANF and PPy and FIG. 26B is a magnified display of the corresponding area in RDG function isosurface map, showing the existence of hydrogen boding and π-π interaction between ANF and PPy;

FIG. 27A is a curve showing a correlation between conductivity and PPy content for ANF-PPy, FIG. 27B is a bar graph showing the conductivity of ANF-PPy under various chemical conditions, FIG. 27C shows the stability of ANF-PPy conductivity upon mechanical bending, FIG. 27D is a schematic of a three-electrode configuration for capacitance characterization, FIG. 27E shows the capacitance of ANF-PPy determined by cyclic voltammetry (CV) and galvanostatic charge/discharge (GCD), FIG. 27F shows curves for different scan rates, FIG. 27G shows capacitance retention during 10,000 charging/discharging cycles, FIG. 27H is a schematic of a two-electrode configuration for capacitance characterization, FIG. 27I shows the capacitance of ANF-PPy characterized by GCD at different current density, FIG. 27J shows gravimetric capacitance at different current density for ANF-PPy with varying thickness and FIG. 27K is a comparison of conductivity and gravimetric capacitance between ANF-PPy and other reported supercapacitor electrodes where the data points were categorized based on Table 1

FIG. 28A shows stress-strain curves for ANF hydrogels with or without PPy composition, FIG. 28B shows the morphology of ANF-PPy network visualized by TEM (left) and a schematic (right) illustrating PPy-reinforced bonding between neighbor ANFs, FIG. 28C shows resistance change under tension for ANF-PPy and PVA-PPy hydrogels, FIG. 28D is a schematic of a kirigami ANF-PPy (1 cm×5 cm) with 17 periodic laser-cut slits where the FEM snapshot shows stress distribution of the kirigami ANF-PPy at 40% tensile strain, FIG. 28E is a typical stress-strain curve for the kirigami ANF-PPy, involving an initial elastic region (I), buckling region (II), and pattern-collapse region (III) and FIG. 28F is a graph of resistance change under tension for the kirigami ANF-PPy;

FIG. 29A is a schematics of the printing process for patterned ANF-PPy on a soft PDMS substrate, FIG. 29B is shows optical images of a representative ANF-PPy pattern with right-angle and curve corners, FIG. 29C shows capacitive bioelectrodes based on two parallel ANF-PPy electrodes of spiral (I) or liner (II) assembly respectively, FIG. 29D is an immunofluorescent staining of cardiomyocytes cultured on ANF-PPy for 5 days. Cardiac-specific proteins of troponin T (red) and connexin 43 (green), and nucleus (blue) were fluorescently labeled, FIG. 29E is an image of calcium transients for cardiomyocytes cultured on PDMS with electrical stimulation and FIG. 29F is without electrical stimulation where traces of fluorescence intensity at random sites were recorded to show the pacing state of cardiomyocytes;

FIG. 30A-D show the steps of solar desalination based on NHSE where FIG. 30A shows that solar desalination based on NHSE eliminates salt accumulation by enhancing mass transfer via highly continuous water channels, FIG. 30B is a schematic of solar desalination using NHSE, FIG. 30C is a scanning electron microscope (SEM) image showing the open porous network of NHSE, FIG. 30D is a schematic illustrating the components of the hybrid nanofiber network with hydrophilic groups for water activation and PPy for photothermal conversion and FIG. 30E is a diagram illustrating the nexus between microstructure and vaporization performance, indicating the coupling of microstructure, water supply, heat transfer, and vaporization enthalpy;

FIG. 31A shows a schematic depiction of the structural characteristics of an NHSE nanofiber network with high ANF content, FIGS. 31B and 31C are SEM images of NHSEs with 2.5% ANF and 5% PVA at scale bar: 5 m and 1 m, respectively, FIG. 31D is a schematic illustration of a hierarchically porous network of NHSEs with low ANF content, FIGS. 31E and 31F are SEM images of NHSEs with 0.5% ANF and 3.4% PVA at scale bars 5 m and 1 m, respectively, FIG. 31G is a sample of NHSE with and without a compressive load of 10 N at a scale bar: 1 cm. and FIG. 31H is a photograph of NHSE samples molded into various shapes at a scale bar: 1 cm.

FIG. 32A shows pore size distribution of NHSEs, FIG. 32B shows the half swollen time and calculated water transport rate of NHSEs, FIG. 32C is a comparison of half swollen time and water transport rate of NHSEs with other reported HSEs, FIG. 32D shows simulations of water pressure in microchannels, FIG. 32E shows water velocity variation in microchannels, FIG. 32F illustrates the simulated average water velocity in NHSEs with different pore sizes, FIG. 32G illustrates the infrared image of NHSEs for the measurement of thermal conductivity, FIG. 32H and FIG. 32I show calculation of thermal conductivity of different NHSEs;

FIG. 33A is a diagram of solar desalination with energy balance and mass transport in a solar desalination system, FIG. 33B is a plot of experimental data (closed symbols) and fitted lines (dotted lines) of pore size, thermal conductivity, and vaporization enthalpy as a function of porosity, FIG. 33C is a plot of experimental and simulated evaporation rates of NHSEs with various porosities, FIG. 33D is a plot of evaporated water mass with various HSEs over 1 h under 1 sun illumination, with a 20% salt solution without evaporator as a control, FIG. 33E shows bar graphs of evaporation rates of different evaporators in various salt solutions under 1 sun, FIG. 33F is a plot of solar evaporation rates of NHSE in 20% salt solution as compared with previous results reported in the literature, FIG. 33G is a plot of stability of different evaporators in 20% brine over 8 h of continuous evaporation where the evaporator based on PVA hydrogels exhibited an obvious decline in evaporation rate, FIG. 33H is a group of photographs of PVA hydrogel and NHSE with continuous evaporation in 20% brine for 12 showing severe salt accumulation on PVA hydrogel while no salt crystal appears on NHSE at a scale bar of 1 cm. and FIG. 33I shows a simulation of salt distribution in NHSEs during desalination in 20% brine under steady state;

FIG. 34A shows the electrical resistance of various salt solutions before and after purification, FIG. 34B shows change in the pH value of acid and alkaline solutions before and after purification, FIG. 34C shows concentrations of four primary ions (Na+, Mg2+, K+, Ca2+) in seawater, before and after desalination where the dotted line indicates the WHO standard for drinking water, FIG. 34D is a UV-vis absorption spectra of dye-contaminated water and purified water, where the inset presents photographs of water samples before and after purification and FIG. 34E shows a proliferation of NIH 3T3 fibroblasts cultured with water purified by NHSEs at scale bars: 200 μm.

FIG. 35A is a schematic of a large-scale KHSE with localized crystallization and dynamic solar tracking, FIG. 35B is an schematic of solar tracking via coupling tilt angles with incident sunlight by tuning the imposed strain at a scale bar of 1 cm., FIG. 35C shows a hydrogel membrane with periodic triangular notches enabling a 3D evaporator array, FIG. 35D shows devices under strain at scale bars of 1 cm and FIG. 35E shows localized crystallization by KHSE with a. scale bar equal to 1 cm;

FIG. 36 is UV-vis-NIR spectrum of ANF-PVA-PPy hydrogel;

FIG. 37A is a schematic of the triangular cuts, including notch length (1), joint width (w), and angle (0) with a ratio between 1 and w between 10:1, FIG. 37B shows stress-strain curves of hydrogel membranes with and without PPy, FIG. 37C shows fracture energy of hydrogel membranes with and without PPy evaluated by tearing test, FIG. 37D is a scanning electron microscopy (SEM) image of the hydrogel, showing an interconnected nanofiber network with a scale bar of 5 m, FIG. 37E shows a stretched KHSE and strain distribution in the equal-scale modelled membrane at 80% strain from FEA simulation with a scale bar: 1 cm, FIG. 37F shows a variation of tilt angle with strain in KHSEs with different θ(the insert image indicates the define of tilt angle), FIG. 37G shows variation of tilt angle in KHSEs with varying modulus at 20% strain. (the insert images demonstrate simulated out-of-plane deformation of samples with modulus of 0.4 MPa (left) and 1 MPa (right)) and FIG. 37H shows a variation of maximum tilt angle of KHSEs with different 1/t where the dotted lines indicate the optimal range;

FIG. 38 is a schematic display of the fabrication process for NHSE;

FIG. 39 shows experimental and FEA images of stretched KHSE with different cut angles from 100 to 40°;

FIG. 40 illustrates stress-strain curves of KHSE with different cut angles from 10° to 40;

FIG. 41A shows the repeatability of deformation of KHSE with a tilt angle scaling of three KHSEs each cut at an angle of 10°, FIG. 41B shows the same at an angle of 20°, FIG. 41C shows an angle of 300 and FIG. 41D shows an angle of 40°;

FIG. 42 shows the effect of modulus on the deformation performance of KHSE (l=10 mm, t=0.2 mm, and θ=200 for KHSEs with a modulus below 0.45 MPa, which are unable to undergo out-of-plane deformation, indicating the modulus threshold;

FIG. 43A shows stress-strain curves for ANF-PVA hydrogels with various during tuning of the ANF/PVA ratio; FIG. 43B shows the effect on the modulus, FIG. 43C shows the effect on solid content of ANF-PVA hydrogels with various component fractions and FIG. 43D shows the deformation of KHSEs based on ANF-PVA hydrogels with 1.2 MPa and 0.2 MPa, respectively, where the KHSE with 0.2 MPa cannot perform out-of-plane due to the low stiffness;

FIG. 44A shows graphs of the effect of the thickness (t) and cut length (l) on the deformation performance of KHSE (θ=20°) where the tilt angle of KHSE (l=10 mm) with varied t and FIG. 44B shows the same parameter where the tilt angle of KHSE has the same l/t;

FIG. 45A illustrates a stretch cycle of KHSE showing negligible hysteresis of tilt angle and FIG. 45B shows high deforming stability of KHSE under 1,000 cycles;

FIG. 46A shows a photograph of KHSE arrays with a scale bar: 1 cm, FIG. 46B is an optical image of a micropatterns on KHSE in the swollen state with a scale bar: 0.5 mm, FIG. 46C shows SEM images of top and side views of micropatterns at a scale bar of 500 m, FIG. 46D shows efficient water transfer on a hydrogel membrane with micropatterns at a scale bar of 1 cm, FIG. 46E is a schematical illustration indicating the mechanism of localized crystallization by KHSE, FIG. 46F shows temperature distribution on KHSE under one sun illumination, FIG. 46G shows sequential photographs displaying a localized crystallization process of KHSE at a scale bar: 1 cm, FIG. 46H is a photograph of localized crystallization on KHSE arrays at a scale bar: 1 cm, FIG. 46I is a graph of mass loss of the KHSE in saturated brine under one sun illumination, with flat evaporator and pure saturated salt solution as controls, FIG. 46J shows long-term evaporation test with saturated brine of KHSE and flat evaporator, and FIG. 46K illustrates the stability of KHSE for treating saturated brine in seven days during evaporation that lasted 8 h per day.

FIG. 47 shows the structural characteristic of a patterned ratchet whose height, width, and pitch are 250 μm and whose tilt angle is 45°;

FIG. 48 shows water transfer on smooth hydrogel membrane;

FIG. 49A shows four temperature measuring positions of KHSE under 1 sun illumination, FIG. 49B shows an infrared image of KHSE and FIG. 49C shows a graph of variation of temperature at different points showing the temperature gradient along KHSE;

FIG. 50 the structure of a test device in which foam floats on a saturated salt solution supported by a floater where KHSE is placed on the foam for the evaporation test;

FIG. 51 shows a series of photographs displaying the dynamic salt removed by reconfiguring the KHSE where the red circle indicates the dropped salt crystal;

FIG. 52 is a Raman spectra indicating free water (FW) and intermediate water (IW) in KHS;

FIG. 53 is a series of photographs of salt accumulation on a flat evaporator;

FIG. 54A shows the effect of solar altitude on evaporation for variations of projected area of a flat evaporator with solar altitude (the insert indicating the projected area, which scales with the sine of solar altitude) and FIG. 54B is a plot of experimental and theoretical evaporation rates varied with solar altitude;

FIG. 55A is a schematic of solar tracking with KHSE by tuning the imposed strain, FIG. 55B is a comparison of projected area using KHSE and flat evaporator (the insert schematic exhibiting the definition of the projected area). FIG. 55C shows photographs of the projected area under different strains, FIG. 55D is a plot of evaporation performance of a flat evaporator, dynamic KHSE for solar tracking, and static KHSE with a tilt angle of 30°, FIG. 55E is a color map showing the variation of tilt angles in a 1×8 KHSE under 40% strain and FIG. 55F shows uniformity of KHSE under strain as the function of column number and row number;

FIG. 56 is a plot of variation of solar altitude in Hong Kong collected between 8 am and 16 μm on the 15th of each month from the Hong Kong Observatory;

FIG. 57 shows optical photographs captured in the direction perpendicular to the tilt angle to measure the projected area showing that as the stretching increases, the projected area remains constant in the longitudinal direction while contracting laterally.

FIG. 58 illustrates the coupling between the tilt angle of KHSE and the solar altitude for maximum evaporation;

FIG. 59. shows that the uniformity decreases with the increase of column number and that some cuts do not deform or deform nonuniformly at both ends of KHSE;

FIG. 60 is a color map showing the distribution of tilt angle in 2×8 and 3×8 of KHSE;

FIG. 61 shows the calculation of reduced projected area with angular deviation;

FIG. 62A to FIG. 62C show ambient conditions of outdoor tests, solar altitude and solar azimuth between 8:00 to 16:00, FIG. 62D to FIG. 62F show tilt angle and imposed strain of KHSE during the evaporation. and FIG. 62G to FIG. 621 show solar intensity, temperature, and humidity change during outdoor test; and

FIG. 63A is a schematic of solar trajectory where a and ρ are solar altitude and solar azimuth, respectively, FIG. 63B is a photograph of the prototype including an evaporation system, a single-chip microcomputer (SCM), two steering engines, and a battery (the insert photograph shows the KHSE), FIG. 63C is a time sequence of photographs displaying the solar tracking system from 8:00 to 16:00 (the insert photographs indicate the state of KHSE), FIG. 63D shows a variation of solar altitude and solar azimuth with time, FIG. 63E shows changes of solar intensity, temperature, and humidity with time and FIG. 63F is a bar graph of water yield of dynamic KHSE, static KHSE, and flat evaporator.

### DETAILED DESCRIPTION

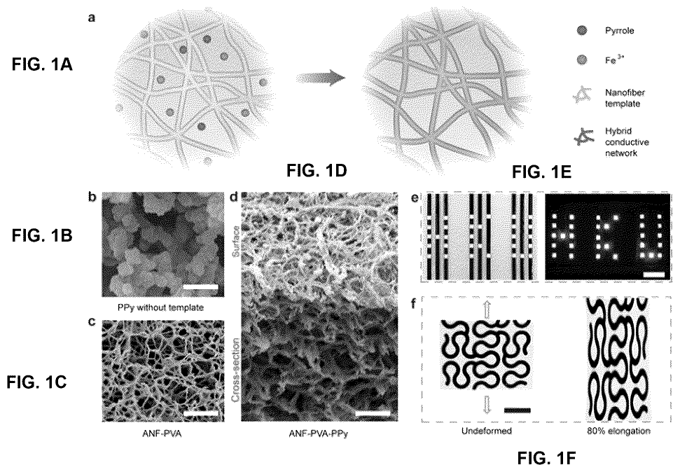

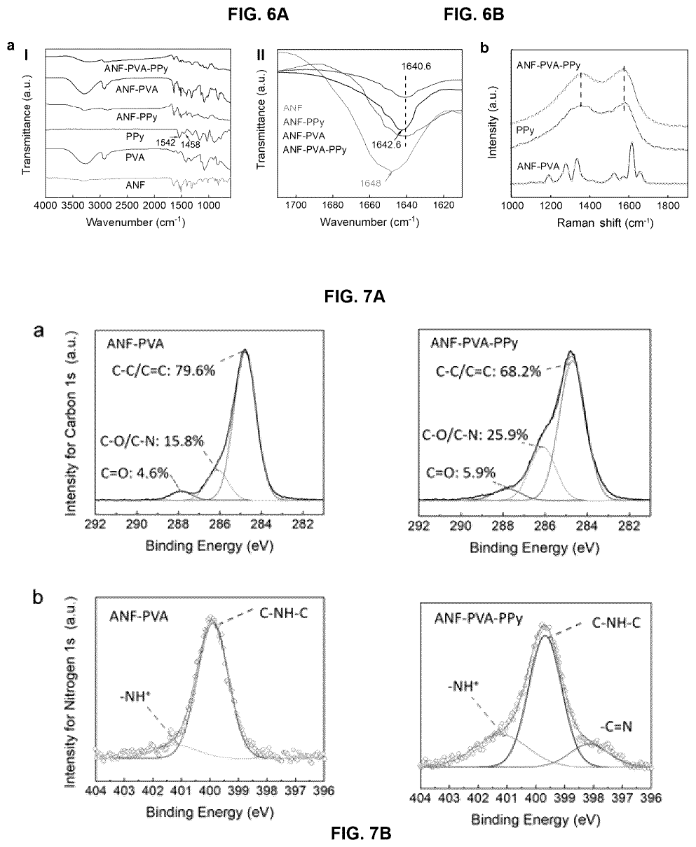

As disclosed by the present invention, the fabrication of conductive nanofiber hydrogels (CNHs) exploits self-assembled 3D networks involving aramid nanofibers (ANFs) [28]. Dispersing ANFs in dimethyl sulfoxide (DMSO) followed by solvent exchange with water generates hydrogels with highly connective 3D fibrillar networks, serving as templates for the assembly of conducting polymers. Incorporating polyvinyl alcohol (PVA) during the processing of hydrogels helps to weld the fibrillar joints via extensive hydrogen bonding, providing enhanced mechanical strength for the nanofiber network [29,30]. Next, monomers (e.g., pyrrole, PPy) are infiltrated into the nano-porous hydrogels in an aqueous media (FIG. 1A), followed by polymerization with added oxidants (e.g., FeCl₃). Interestingly, this facile process leads to the fabrication of hybrid nanofiber network with very efficient electrical percolation for the synthesized conducting polymers (i.e., polypyrrole, PPy). As indicated by scanning electron microscopy (SEM), PPy tends to form randomly distributed particles in an aqueous media without the nanofiber template (FIG. 1B). On the other hand, in the presence of ANF-PVA (FIG. 1C), the synthesized PPy conforms to the nanofiber framework with identical network topology (FIG. 1D where the scale bars equal 1 m. Successful integration of PPy on the nanofibers was also confirmed with Fourier transform infrared spectroscopy (FTIR), Raman spectroscopy, and X-ray photoelectron spectroscopy (XPS) (FIGS. 6A to 7B). Indeed, the distinct percolation process for PPy imparts high electrical conductance to the CNHs (FIG. 1E). As shown in FIG. 6, for ANF-PPy and ANF-PVA-PPy, characteristic peaks for C═C and ═C—N symmetric and asymmetric ring stretching were measured at 1542 and 1458 cm⁻¹ respectively, demonstrating the successful integration of PPy. For pure ANF, the aramid C═O stretching band peaked at 1648 cm⁻¹. ANF-PVA has a red-shift of this peak to 1642.6 cm⁻¹, reflecting the effects of hydrogen bonding between ANF and PVA. A larger scale of red-shift to 1640.6 cm⁻¹ was found in ANF-PPy composites, which indicates hydrogen bonding between ANF and PPy as well.

In FIG. 7 the C is spectra have 3 peaks at binding energies of 284.8, 286.1, and 287.9 eV, corresponding to C—C/C═C, C—O/C—N, and C═O, respectively. The presence of PPy is indicated by the increase of peak intensity at 286.1 eV. For N is spectra, 3 peaks at 398.1, 399.6, and 401.2 eV represent —N═C, C—NH—C, and —NH+ respectively. The emerging peak at 398.1 eV corresponding to —N═C further confirms the incorporation of PPy into ANF-PVA-PPy. The significant increase of the peak intensity at 401.2 eV also demonstrates peroxidation of PPy in the presence of excess oxidant of FeCl₃.

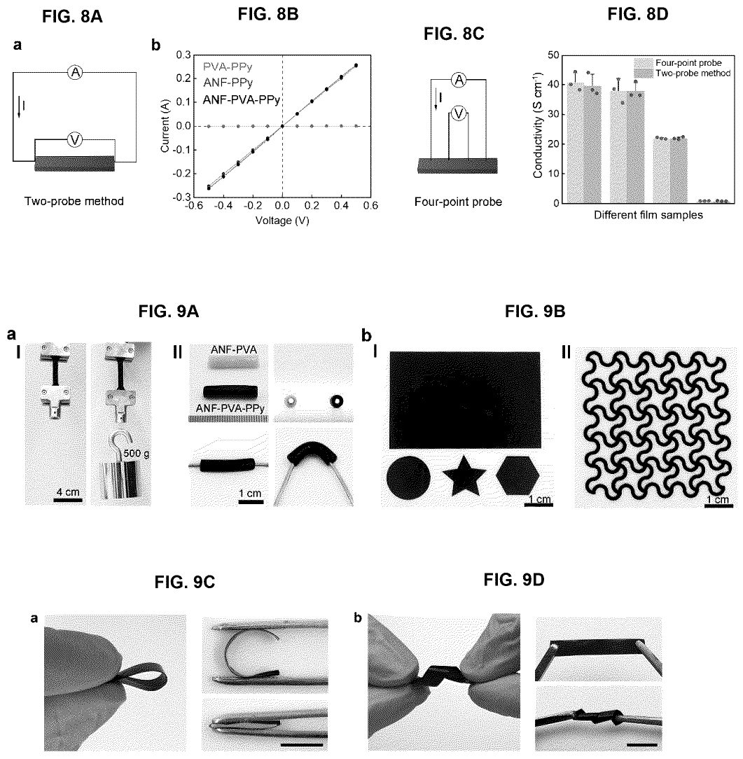

The effective transport of electrons in CNHs is also indicated by its linear I-V characteristics (FIG. 8A to 8D). Furthermore, patterning of CNHs was made possible via spatially selective synthesis of PPy in the ANF-PVA hydrogel matrix, which can generate electrodes and interconnects for soft electronic devices. The robustness of the 3D fibrillar network was translated into the macroscopic behaviors of CNHs, as exemplified by the high mechanical strength of bulk samples (FIG. 9A & 9B) and stretchability of hybrid serpentine structures (FIG. 1F where the scale bars are 1 cm). FIG. 9C shows photographs of ANF-PPy under substantial bending and FIG. 9D shows substantial twisting (b). Scale bar: 1 cm.

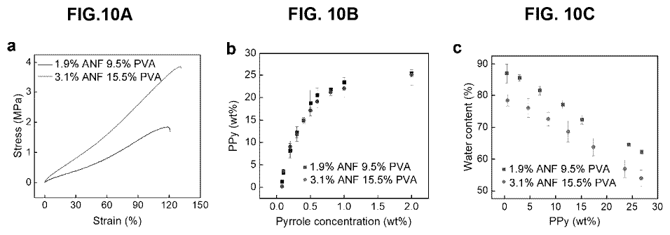

The effect of ANF networks on the electrical percolation behaviors of PPy was investigated. This provided quantitative guidance for the tuning of conductivity for CNHs. Specifically, a series of CNHs with various PPy contents were synthesized by varying the concentration of the monomer (Py) during the polymerization. As shown in FIG. 10 the PPy content increased with the increase of pyrrole concentration until 1 wt % and then reached a plateau. Both the CNHs prepared from 1.9% ANF, 9.5% PVA and 3.1% with ANF 15.5%-PVA matrices demonstrate linear decrease in water content with increasing PPy content.

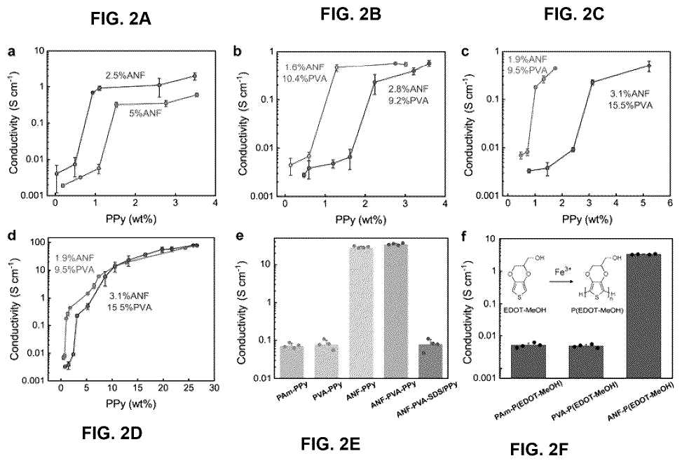

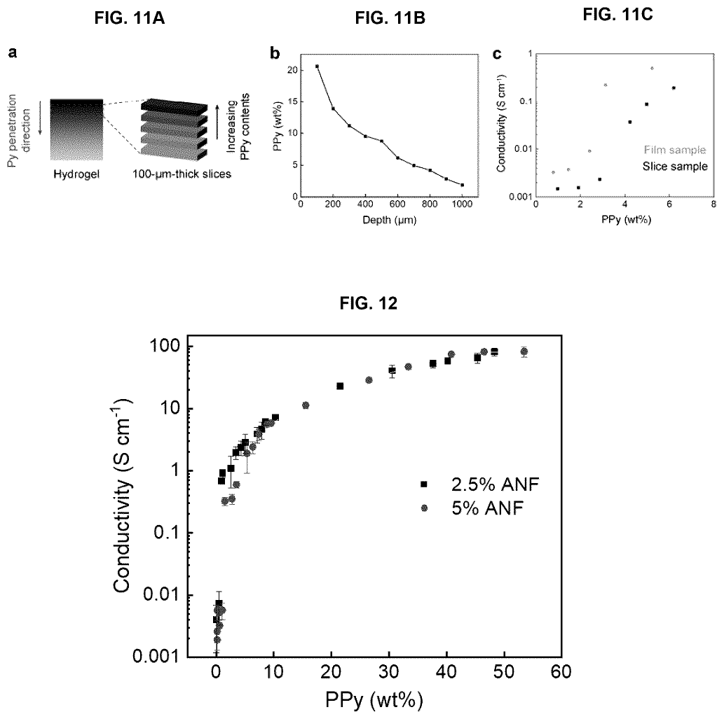

The electrical conductivity of CNHs is closely related to the content of PPy, showing an abrupt increase at the threshold fraction of PPy corresponding to its electrical percolation (FIG. 2A). The conductivity of CNHs and the percolation threshold for PPy are also dependent on the various configurations of the nanofiber templates (FIG. 2A-2C), providing further information about the assembly of the microscopic materials. To minimize artifacts induced by the non-uniformity of PPy distribution within the sample, hydrogel films were used with a ˜150 μm thickness for the synthesis of PPy and the characterization of CNHs. For these thin-film samples, the concentration gradients for monomers and oxidants during the synthesis only led to small spatial variation of the PPy content, which agreed with the results from precise characterization on sections of bulk CNH samples (FIG. 11A-11C). The graphs in FIG. 11 was made from a sample obtained from Py (0.4 M), FeCl₃ (0.48 M), and 3.1% ANF-15.5% PVA matrix (18 mm in diameter and 8 mm in height) during the synthesis. FIG. 11C is a graph of the electrical conductivity as a function of PPy content for CNHs with 3.1% ANF-15.5% PVA matrix. The information of FIG. 11C is obtained from both slice samples and film samples. The results show consistency of measured threshold for electrical percolation. The lower absolute values of conductivity for slice samples may be attributed to the structural defects introduced during the mechanical cryo-sectioning process.

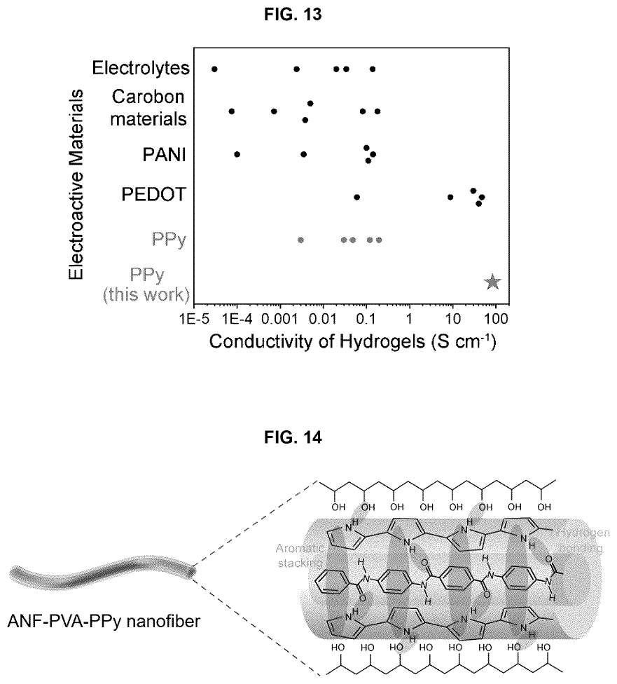

A unique feature of CNHs is that the PPy assembled into percolated conduction pathways at a very low volume fraction, which is guided by the highly connected nanofiber templates. To validate this point, a dependence of electrical percolation threshold in CNHs on the solid content of ANFs was observed. Specifically, the percolation threshold for PPy in CNHs with 5 wt % of ANFs is slightly above 1 wt %. In other CNHs with a lower content of ANFs (2.5 wt %), the percolation threshold for PPy dropped to a level below 1 wt % (FIG. 2A and FIG. 12). In FIG. 12 the electrical conductivity of ANF-PPy with a sparser nanofiber template (2.5% ANF) is higher than that of 5% ANF before the PPy content reaches 10 wt %. Other samples showed similar electrical conductivity under the same PPy contents. This phenomenon can be understood as the sparser fibrillar network requires less PPy to form a continuous coating, which corresponds to a lower threshold for the formation of conduction pathway. Similar trends were also observed in various CNHs with incorporated PVA content (FIGS. 2B and 2C). On the other hand, for the composite ANF-PVA templates, a higher amount of PPy is required to achieve electrical percolation as compared to those with pure ANFs, partly due to the higher solid content of the composite templates and the interactions between PPy and PVA. As the content of PPy increases the conductivity of CNHs steadily increases and finally reaches an unusually high level of ˜80 S cm⁻¹ (FIG. 2D). Indeed, this value was not achieved by other existing conductive hydrogels (FIG. 13 in which data points were categorized based on the electroactive components in the hydrogels. See Table 1.

TABLE 1
Summary of conductivities of other reported
electroconductive hydrogels.

| | | | Conduc- | |
|---|---|---|---|---|
| | | | tivity | |
| Electroactive Materials | | Hydrogel Matrix | (S cm⁻¹) | Ref. |
| Electrolytes | NaCl | Hydroxypropyl | 0.034 | 1 |
| | | cellulose/PVA | | |
| | LiCl | PAAm | 0.139 | 2 |
| | sodium | Aramid nanofiber/PVA | 0.02 | 3 |
| | citrate | | | |
| | Na₂SO₄ | PEG | 0.0024 | 4 |
| | choline | Poly α-lipoic acid | 0.00003 | 5 |
| | chloride | | | |
| Carbon | GO | GO | 0.005 | 6 |
| materials | graphene | PAAm | 0.000075 | 7 |
| | graphene | Collagen | 0.0038 | 8 |
| | rGO | Polydopamine/PAAm | 0.18 | 9 |
| | CNTs | P(AAm-AAc) | 0.082 | 10 |
| | CNTs | Chitosan/Gelatin | 0.00072 | 11 |
| PANI | PANI | Chitosan/PEG/PANI | 0.0035 | 12 |
| | PANI | PVA/PANI | 0.1 | 13 |
| | PANI | PSS/PANI | 0.142 | 14 |
| | PANI | Phytic acid/PANI | 0.11 | 15 |
| | PANI | N-fluorenylmethoxy- | 0.0001 | 16 |
| | | carbonyl | | |
| | | diphenylalanine/PANI | | |
| PEDOT | PEDOT | PEDOT/PSS | 8.8 | 17 |
| | PEDOT | PEDOT/PSS | 47.4 | 18 |
| | PEDOT | PEDOT/PSS | 40 | 19 |
| | PEDOT | PEDOT/sulfonated | 0.06 | 20 |
| | | lignin/PAAc | | |
| | PEDOT | PU/PEDOT/PSS | 30 | 21 |
| PPy | PPy | Agarose | 0.195 | 22 |
| | PPy | Silk fibroin | 0.03 | 23 |
| | PPy | PAAm | 0.12 | 24 |
| | PPy | Cellulose | 0.048 | 25 |
| | | nanofibers/PVA | | |
| | PPy | PAAm/Chitosan | 0.003 | 26 |

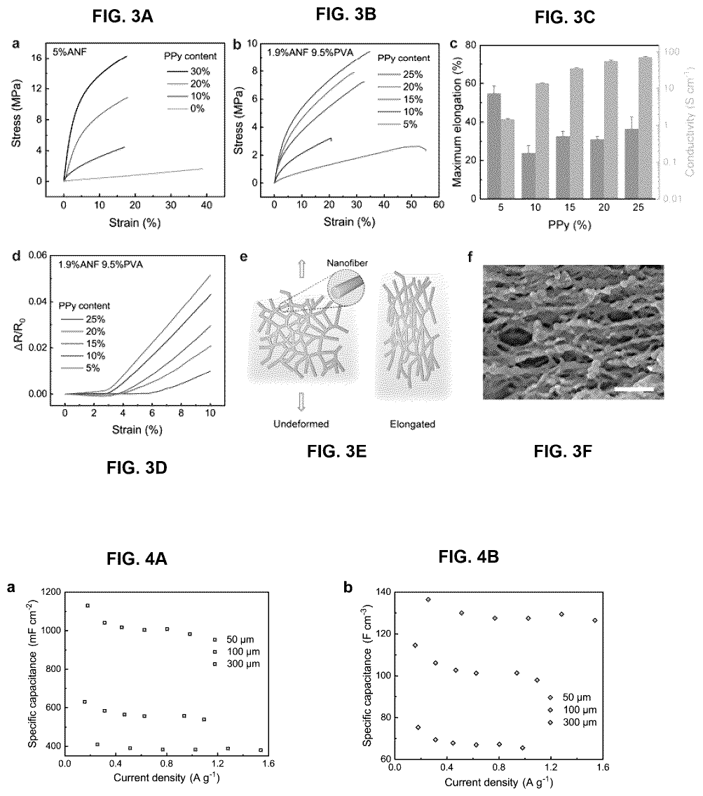

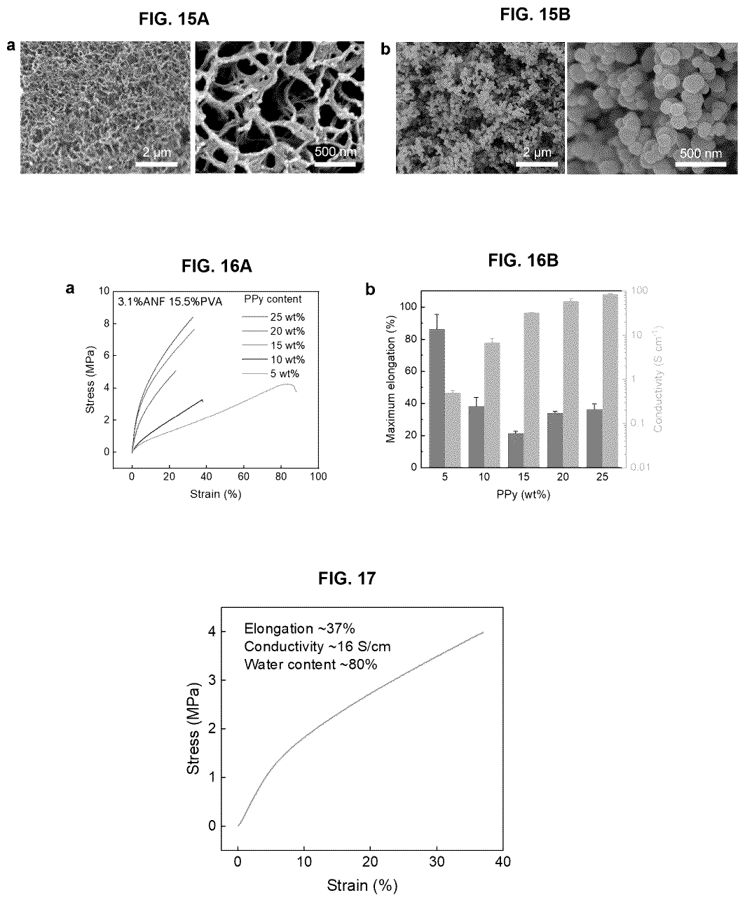

The templating effect in CNHs can be attributed to the interactions between ANFs and conducting polymers. In particular, the aromatic rings and amide groups on ANFs afford attractions to PPy via π-π stacking and hydrogen bonding (FIG. 14). Hydrogen bonding is also possible between PPy and PVA, as well as PVA and ANF. Although further details of the intermolecular interactions in ANF-PVA-PPy composites are difficult to characterize, a series of controlled experiments demonstrated the unique templating effects arising from ANFs. Under similar conditions for the synthesis of PPy, replacing ANFs with other hydrogel matrices, such as those from pure PVA or polyacrylamide (PAm), led to a reduction of conductivity by three orders of magnitude as compared with those involving ANF templates (FIG. 2E). Microstructural examination also revealed randomly distributed PPy particles in the hydrogel matrix without ANFs, which contrasts with the composite fibrillar network in CNHs (FIG. 15). In another controlled experiment, adding sodium dodecyl sulfate (SDS) during the polymerization of PPy also led to a dramatic reduction in conductivity despite the existence of an ANF template. This phenomenon occurs because the added surfactants disrupted the intermolecular interactions between ANFs and PPy, which compromised the formation of percolated conduction pathways. Furthermore, the templating effects from ANFs are also applicable to other conducting polymers such as derivatives of PEDOT. Specifically, hydroxymethyl functionalized PEDOT synthesized in the presence of ANFs led to a high conductivity of the composite hydrogel, which is three orders of magnitude greater than those without templating by ANFs. FIG. 2F shows the conductivity of ANF-P(EDOT-MeOH) as compared with hydrogels prepared with pure PVA or PAm matrix. On the other hand, a similar templating effect for conducting polymers was also observed in other nanofiber networks such as those from bacterial cellulose (FIGS. 3B and 3C), suggesting a general strategy for the synthesis of electroconductive composites. Such networks could be covered with PPy.

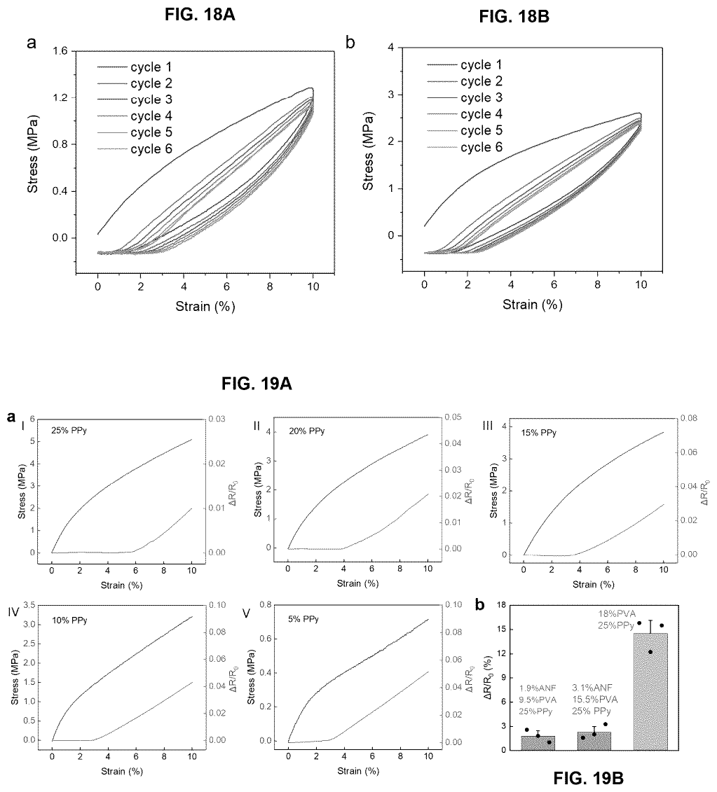

The hybrid assembly of a polymeric nanofiber network not only imparts high electrical conductivity, but also provides structural robustness of CNHs under mechanical loading. The ANF-PPy hydrogels exhibit high strength ranging from 1.6 MPa to 17.6 MPa depending on the solid content of PPy (FIG. 3A). On the other hand, incorporation of PPy led to a decrease of failure strain for the hydrogel partly due to the intrinsic molecular rigidity of the conducting polymer. Enhancing the deformability for CNHs, including flexible PVA chains in the hydrogel matrix, becomes advantageous. Indeed, the ANF-PVA-PPy hydrogels exhibit a stretchability significantly higher than that of ANF-PPy (FIG. 3B in which the scale bar is 500 nm and FIG. 16). For ANF-PVA-PPy hydrogels, as the content of PPy increases from 5% to 15%, the maximum elongation first dropped from 55% to 20% and then increased to the level of ˜34%. It is possible that incorporation of PPy interfered with the interactions between ANF and PVA which contributed to the stretchability of the original fibrillar network. As the fraction of PPy keeps increasing, deformation of the composite hydrogel becomes more dependent on the behavior of PPy along with its interactions with ANF and PVA. In this regard, further increase of its content enhanced intermolecular interactions as well the deformability of the hybrid network. Generally, the conductivity and mechanical characteristics of CNHs can be tuned by varying the solid content (FIG. 3C and FIGS. 16-18), providing designing flexibility for functional devices. In an optimized configuration, the hydrogel shows a stretchability of 37%, conductivity of 16 S/cm, and water content of 80%. The hydrogel can be synthesized based on 1.9% ANF, 9.5% PVA matrix and 0.3 wt % pyrrole, polymerized under pH 7 and 0° C. for 2 h.

The high robustness of CNHs is also exemplified by their strain-invariant electrical properties. Specifically, ANF-PVA-PPy samples exhibit negligible changes in resistance within a range of imposed elongation (up to ˜3%-6%) (FIG. 3D and FIG. 19A). In FIG. 19A the stress and change of resistance (ΔR/R₀) are recorded simultaneously under imposed tensile strain, showing strain-invariant conductance within the initial range of deformation. The negligible change in resistance can be attributed to the re-orientation of the hybrid nanofiber network, which can accommodate macroscopic stretching without altering the topology of the conduction pathway (FIG. 3E). From SEM characterization on stretched samples, the nanofiber network exhibits significant orientation according to the mechanical load (FIG. 3F), which demonstrates the microstructural reconfiguration that contributed to the strain-invariant resistance of CNHs. As the elongation exceeds the initial regime, CNHs exhibit increases in resistance with imposed strain due to successive breakage of the conduction pathway. Nevertheless, the slopes for CNHs (0.2-0.7) are much smaller than those of typical conductive nanocomposites (e.g., 1.5-1.8 for PVA-PPy) (FIG. 19B). Indeed, the stability of electrical conductance under deformation makes CNHs advantageous for diverse applications as stretchable electrodes and/or interconnects.

### Soft Bioelectronic Interfaces/Electrodes

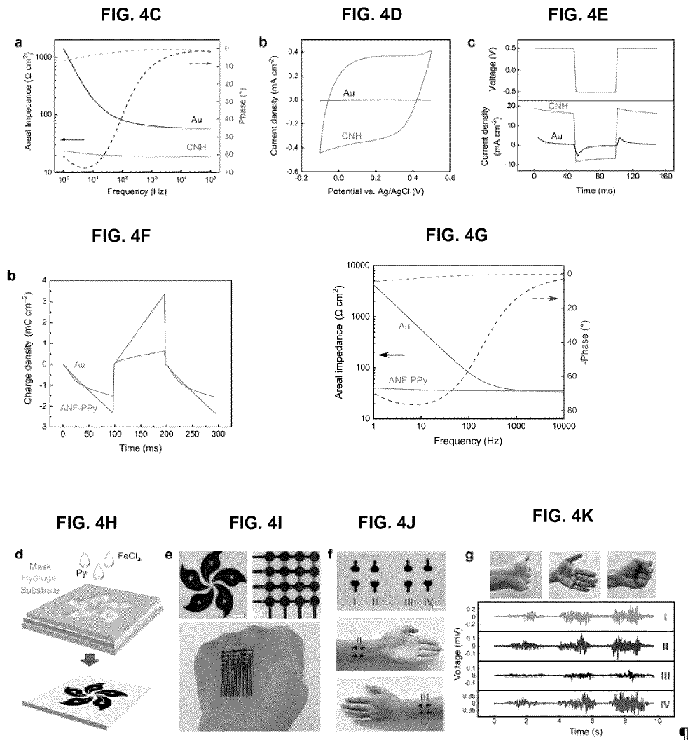

The CNHs have significant applications as soft bioelectronic interfaces. The electrochemical impedance per unit area for CNHs (3.1% ANF, 15.5% PVA, 25% PPy) in phosphate buffered saline (PBS) is 67.2% to 98.3% lower than that of thin-film gold (Au) electrode in the physiologically relevant frequency range. FIG. 4C is a graph of electrochemical impedance spectroscopy (EIS) of CNH and an Au electrode. FIG. 4G shows areal impedance of the ANF-PPy and an Au foil. The low impedance of CNHs originates from their extensive nanostructures providing high interfacial capacitance. The areal and volumetric capacitances of ANF-PPy normalized with area are shown in FIG. 4A and normalized with volume are shown in FIG. 4B. These features are beneficial for precise characterization of various bioelectric activities with low signal amplitude. CNHs also show high capacities in storing (FIG. 4D) and injecting (FIG. 4E) electric charges as compared with those of Au electrodes, which are desirable for delivering electrical stimulations to biological tissues. Table 2 shows the performances of supercapacitor electrodes according to the present invention.

TABLE 2
Performance of supercapacitor electrodes

| | | | | | Scan rate or | | | |
|---|---|---|---|---|---|---|---|---|
| | | C_{A} | C_{V} | C_{M} | current | | Conductivity | |
| Electrode | Electrolyte | (mF cm⁻²) | (F cm⁻³) | (F g⁻¹) | density | | (S cm⁻¹) | Ref. |
| Graphite | LiClO₄ | | | 40 | 0.1 | A g⁻¹ | 20 | 8 |
| Graphite oxide | EMIM TFSI | | 60 | 166 | 2.8 | A g⁻¹ | 5 | 9 |
| CNT | PVA—H₃PO₄ | 72.9 | 19 | 42.4 | 0.29 | A g⁻¹ | | 10 |
| CNT/MnO₂ | PVA—LiCl | 888.7 | 154.7 | | 2.3 | mA cm⁻² | 29 | 11 |
| MXene/PPy/PVA | PAAm—Zn(CF₃SO₃)₂ | 195 | | 49.1 | 0.2 | mA cm⁻² | 253 | 12 |
| PANI/MnO₂ | H₂SO₄—Na₂SO₄ | | | 224 | 1 | A g⁻¹ | | 13 |
| PEDOT/graphene | Na₂SO₄ | | | 104 | 0.5 | A g⁻¹ | 0.73 | 14 |
| PEDOT—PSS/CNT | NaNO₃ | | | 104 | 0.2 | A g⁻¹ | 20 | 15 |
| PEDOT—PSS | PVA—H₂SO₄ | 115 | 202 | | 0.54 | A cm⁻³ | 8.8 | 16 |
| PPy/PAAm/CMC | PVA—NaCl | | | 126.4 | 0.8 | A g⁻¹ | 0.1 | 17 |
| PPy/Au particle/CNT/PAAm | PAAm—Na₂SO₄ | 885 | | | 1 | mA cm⁻² | 0.03 | 18 |
| ANF—PPy (62.4) | H₂SO₄ | 409.2 | 136.4 | 241.3 | 0.3 | A g⁻¹ | 106 | |
| ANF—PPy (46.3) | H₂SO₄ | 583.6 | 106.1 | 209.7 | 0.3 | A g⁻¹ | 72 | This |
| ANF—PPy (35.8) | H₂SO₄ | 1040.5 | 69.4 | 213.7 | 0.3 | A g⁻¹ | 43 | work |

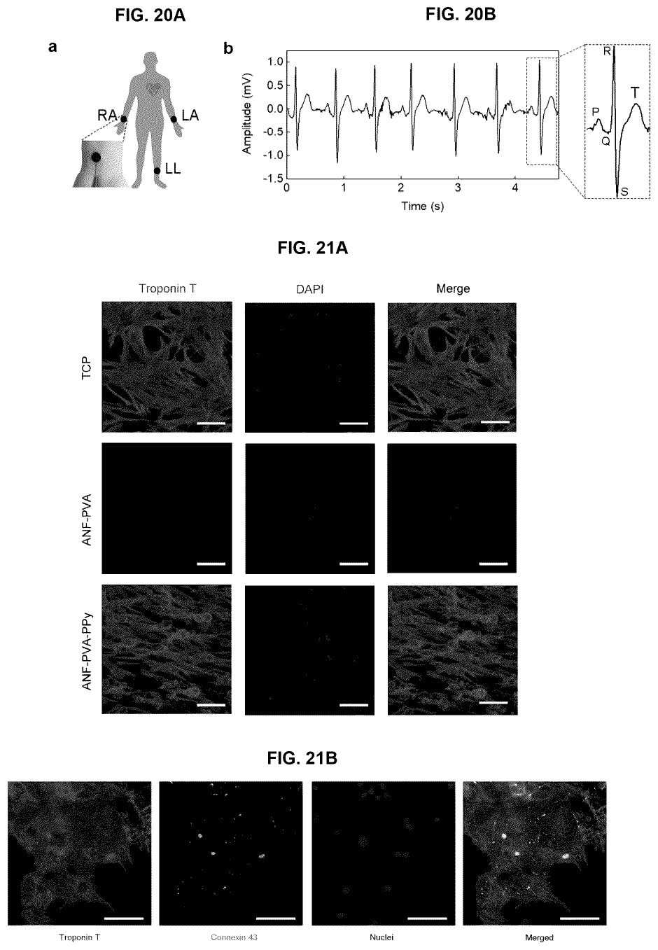

FIG. 4F shows a graph of cyclic electrochemical current pulse injection curves of electrodes based on ANF-PPY and an Au foil with a square wave between −0.5 V and 0.5V. In FIGS. 4D and 4F all electrodes have the same thickness of 100 μm and were tested in PBS using an Ag/AgCl electrode as a reference. To generate patterns of CNHs as bioelectrodes, ANF-PVA hydrogel samples were masked with waterproof adhesive tapes and then treated with Py and FeCl₃ solutions (FIG. 4H). The synthesized PPy is incorporated into the ANF-PVA matrix only in the area exposed by the mask, leading to custom patterns of CNH (FIG. 4I in which the scale bar is 1 cm). The hybrid hydrogel membranes involving CNH electrodes were laminated onto the skin for the measurement of electromyogram (EMG) (FIG. 4J in which the scale bar is 1 cm and FIG. 4K) or electrocardiogram (ECG) (FIGS. 20A and 20B), which captured high-quality electrophysiological signals. Distinct hand motions were characterized with EMG recordings from four independent CNH electrodes mounted on the forearm, proving a means for human-machine interactions. See FIGS. 4J and 4K.

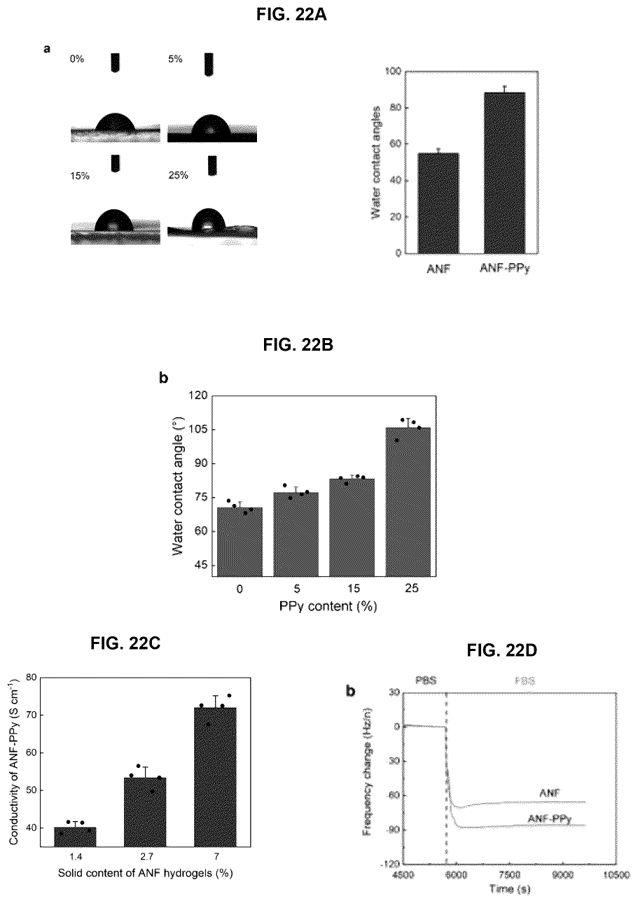

Finally, CNHs show great potential for modulating the behaviors of electrogenic cells. In pilot experiments carried out using the present invention, neonatal rat cardiomyocytes were seeded on CNH samples (3.1% ANF, 15.5% PVA, 25% PPy), as well as ANF-PVA and tissue culture plates (TCPs) for comparison. After five days of cultivation, cardiomyocytes showed significant spread on the surface of CNHs and TCPs, while ANF-PVA led to little attachment of cells (FIG. 21A). FIG. 21B shows immunofluorescent staining of cardiomyocytes cultured on ANF-PPy after 5 days. Troponin T, connexin 43, and nuclei of cardiomyocytes were fluorescently labeled in red, green, and blue respectively. The scale bar is 50 μm. The improved cell adhesion on CNHs can be attributed to the decreased hydrophilicity induced by PPy, as well as interactions between its positively charged surface and negatively charged cells [31]. FIG. 22A shows photos of water droplets (3 μl) on the surfaces of ANF-PVA-PPy (3.1% ANF, 15.5% PVA) with various PPy contents after 10s contact showing the wettability of ANF-PVA-PPy with different PPy contents and a bar graph of water contact angles as a function of ANF and ANF-PPy. FIG. 22B is a bar graph of water contact angles as a function of PPy content and FIG. 22C is a bar graph showing a correlation between the solid content of ANF hydrogels and the conductivity of resulting ANF-PPy hydrogels. FIG. 22D shows the differences in the QCM frequency change as a function of time for ANF and ANF-PPy hydrogels after contact with fetal bovine serum (FBS);

In effect, hydrophobicity is induced by PPy, which is indicated by the water contact angles calculated according to photographs of water droplets (3 l) on the surfaces of ANF and ANF-PPy after 10 s contact. FIG. 22D shows the differences in the QCM frequency change as a function of time for ANF and ANF-PPy hydrogels after contact with fetal bovine serum (FBS). The decrease of QCM frequency is proportional to the increase of hydrogel mass from the protein absorption.

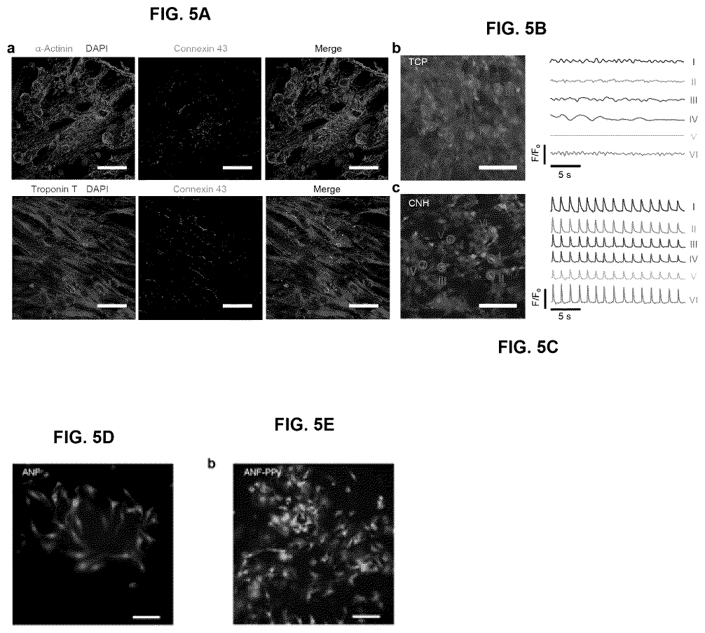

The cardiomyocytes grown on CNHs exhibit fast maturation indicated by good cell alignment [5,32] and pronounced expression of cardiac-specific proteins (FIG. 5A and FIG. 21 with the scale bar at 50 μm), where α-actinin and troponin T are responsible for myocardial contraction and connexin 43 is involved in gap junctions for electrical and mechanical coupling [33]. In FIG. 5A α-actinin is shown in purple; connexin 43 in green; troponin T in red) and the nucleus (in blue) for neonatal rat cardiomyocytes cultured on CNH (3.1% ANF, 15.5% PVA, 25% PPy. The cells are well attached to ANF-PVA-PPy substrates, showing good alignment and expression of cardiac-specific protein troponin T. Furthermore, calcium transient imaging revealed distinct contractile behaviors of the cardiomyocytes cultured on the CNHs as compared to those on TCPs. Specifically, cardiomyocytes cultured on rigid and non-conductive TCPs showed weak and random excitation without coordination between cells (FIG. 5B). In contrast, cells attached on CNHs exhibit spontaneous and synchronous excitation with the electromechanical conduction facilitated by the soft and conductive CNH substrate (FIG. 5C with a scale bar of 100 μm1). Coordinated contraction of cardiomyocytes on CNHs also led to macroscopic actuating of the liquid media, mimicking the behavior of beating myocardium. FIG. 5D shows cytocompatibility of hydrogels with live-dead staining of cardiomyocytes cultured on ANF and FIG. 5E shows staining on ANF-PPy hydrogels after 5 days; Live cells fluoresce in green, and dead cells fluoresce in red. Scale bar: 100 μm;

Methods for preparing conductive hydrogels are as follows. ANF-PVA hydrogels are fabricated according to a previously reported method [29]. Briefly, Kevlar para-aramid pulp (Type 979; DuPont) and poly (vinyl alcohol) (PVA; Mw: 146,000-186,000; 99%+hydrolysed; Sigma-Aldrich) are dissolved in dimethyl sulfoxide (DMSO) under magnetic stirring at 95° C. for 7 days. The resulting ANF and PVA liquids are mixed and then poured into a mold or cast on a flat steel plate using a film coater. Bulk and film hydrogels are achieved by solidification of an ANF-PVA mixture through solvent exchange in deionized (DI) water for 24 h.

For comparison, pure ANF, PVA, or polyacrylamide (PAm) hydrogels were also prepared. ANF hydrogels were prepared from an ANF dispersion solidified by solvent exchange in DI water. PVA hydrogels were achieved using PVA solution (15% in DI water) undergoing a freeze-thaw cycle with 24 h freezing at −20° C. and thawing to room temperature. PAm hydrogels was synthesized by polymerization of acrylamide (3 M) using crosslinker N,N′-methylenebisacrylamide (2 mM), initiator potassium persulfate (3 mM) and accelerator N,N,N′,N′-tetramethylethylenediamine (1.8 mM) in deaired DI water at room temperature for 2 h. Synthesis of ANF-PVA-PPy was performed by pre-soaking ANF-PVA hydrogel in a pyrrole (Sigma-Aldrich) solution under ice-water bath with vibration at 160 rpm by a shaker. After 1 h of pre-soaking, FeCl₃ was added into the solution and the polymerization proceeded for 2 h. Samples were soaked in 0.5 mM FeCl₃ solution to preserve their high electrical conductivity.

For further comparison, ANF-PPy, PVA-PPy and PAm-PPy were fabricated using the same procedures for the polymerization of PPy. Then ANF-PVA-SDS/PPy was fabricated with additional SDS (0.1 M in concentration) added into the aqueous solution during the polymerization. For the synthesis of PEDOT-incorporated composite hydrogels, samples were soaked in EDOT-MeOH solution (2.5 wt % in water) for 1 h. Next FeCl₃ (1.2 times molarity of EDOT-MeOH) was added to trigger the polymerization at room temperature for 24 h with vibration at 160 rpm by a shaker.

SEM examination (Hitachi S4800 FEG) was used to characterize the structure of the various hydrogels. In particular, hydrogels were cut to expose cross-sections after plunge-freezing in liquid nitrogen and dried by critical point drying (CPD; Tousimis Autosamdri 931). The chemical structures of samples were investigated by Fourier Transform Infrared (FTIR) (Thermo Fisher IS50), Raman spectroscopy (DXRxi, Thermo Fisher), and XPS (K-Alpha, Thermo Fisher).

Electrical conductivity was measured by the two-probe method (Keithley 2450) or a standard four-point probe (HPS2661, Helpass electronic technologies) (FIG. 8). Using the two-probe method, bias voltage over the range from −0.5 to 0.5 V was applied on both ends of cuboid hydrogels with fixed length (L) of 25 mm, width (W) of 4 mm and thickness (T) of 0.15 mm, unless stated otherwise. The conductivity (κ, S cm-1) was calculated by the following relationship:

$$κ = ( \frac{I}{V} ) ( \frac{L}{W \times T} ) \qquad ( 1 )$$

where V is the voltage, and I is the current. A four-point probe was applied on square film samples with side length of 30 mm and thickness less than 0.15 mm. To study the distribution of PPy and the gradient of conductivity in a thick hydrogel, an 8 mm-thick ANF-PVA-PPy sample was cut into slices that were 100 μm in thickness by a microtome cryostat (HM525 NX, Thermo Fisher) from the top surface downwards. The conductivity of each slice was tested and the PPy content of each slice was determined with an organic elemental analyzer (Unicube, Elementary). Alternatively, to confirm the PPy content in the hydrogels, the conductive hydrogel was first weighed and then dried completely in a vacuum oven at 80° C., followed by measurement of the weight again. The solid content of the hydrogel matrix was measured before PPy loading. Then the PPy content was calculated by:

$${C}_{PPy} = \frac{{W}_{dry} - {W}_{ANF - PVA}}{{W}_{wet}} \times 100 \% \qquad ( 2 )$$

where CPPy is the weight fraction of PPy in the CNH, Wwet is the weight of the CNH in hydrated state, Wdry is the weight of the CNH after complete dehydration, and WANF-PVA is the dry weight of the hydrogel matrix.

For the measurement of resistance change of CNHs under applied strain, samples with a size of 25 mm×4 mm×0.15 mm were loaded on a tensile-compressive tester (Zwick Roell) at a deformation rate of 1% s-1. Meanwhile the resistance of the conductive hydrogel was recorded until fracture.

Electrochemical impedance spectroscopy (EIS) of CNHs was recorded with a sine wave of amplitude at 10 mV. Cyclic voltammetry was performed with a scan rate of 0.15 mV s-1. Square-wave voltage pulses were applied on CNHs from −0.5 to 0.5 V each with a duration of 50 ms and the corresponding current was simultaneously recorded. All the electrochemical tests were conducted on CNHs or Au foil with an exposed area of 1 cm2 in PBS, using Pt foil as a counter electrode and silver/silver chloride (Ag/AgCl) as a reference electrode by an electrochemical workstation (PGSTAT302N, Metrohm Autolab).

Mechanical tests were performed on a tensile-compressive tester (Zwick Roell). CNHs with a sample of size 25 mm×4 mm×0.15 mm were loaded with a deformation rate of 1% s-1.

The devices of the present invention can be used to record electrophysiological characteristics. Three CNH electrodes with identical geometry were used to detect ECG signals (FIG. 20A). The electrodes were adhered to a volunteer's left-forearm, right-forearm, and left leg by conductive gel (SignaGel Electrode Gel). For EMG measurement, patterned CNH electrodes arrays were attached to a volunteer's forearm, and a commercial gel electrode was attached to the elbow as a reference electrode. Electrophysiological signals were all collected by using a commercial data acquisition system (PowerLab T26, AD Instruments). All the physiological experiments were standardized.

For cell experiments, CNH samples were rinsed thoroughly by PBS and soaked in Dulbecco's modified Eagle medium (DMEM) supplemented with 10% fetal bovine serum (FBS) and 1% penicillin-streptomycin (Gibco). Neonatal rat cardiomyocytes (NRCMs) were harvested from the left ventricles of 2-day-old Sprague-Dawley rats as reported previously. [35] Briefly, ventricles were separated and collected from the neonatal hearts. After overnight digestion by 0.1% trypsin (Gibco) at 4° C., tissues were washed with DMEM (Gibco) and further dissociated into single cells using 1 mg mL-1 rat collagenase type II (Worthington) at 37° C. for 20 min. The digestion step was repeated three times until no visible tissues was left, followed with Percoll density-gradient centrifugation for 30 min to remove the non-cardiomyocyte population. After cell counting, 5×105 NRCMs in a volume of 20 μL were seeded onto gelatin-treated [36] CNHs or TCPs as controls. After 30 min incubation, culture medium containing 10% FBS (Gibco) and 1× antibiotic-antimycotic (Gibco) was added.

For immunocytochemistry, the cells were fixed with 4% paraformaldehyde (PFA) for 15 min at 4° C., permeabilized with 0.2% Triton X-100 in PBS for another 15 min at room temperature, and blocked by 3% BSA for 1 h. Subsequently, the cells were incubated with primary antibodies to detect α-sarcomeric actinin (1:100, Sigma-Aldrich) or troponin T (1:100, Thermo Fisher) overnight at 4° C. After washing three times with PBS, the cells were incubated for 1 h at room temperature in the dark with Alexa Fluor 488 conjugated goat anti-rabbit antibody (1:500; Thermo Fisher) or Alexa Fluor 568 conjugated goat anti-mouse antibody (1:500; Thermo Fisher). Cells and then were washed three times before adding DAPI solution (VectaShield) for nuclear staining. Samples were observed under a scanning laser confocal microscope (Eclipse Ni, Nikon).

Intracellular calcium transient imaging was performed using Fluo-4 AM (Thermo Fisher) to label calcium ions in NRCMs. Briefly, 5 days after cell seeding, hydrogels were washed twice with PBS and incubated with the calcium indicator media containing 10 μM fluo-4 AM and 0.1% Pluronic F-127 (Thermo Fisher) in PBS for 30 min at 37° C. Next, the samples were washed with media and observed under an inverted fluorescence microscope (Eclipse Ti2, Nikon).

### Flexible Electrochemical Electrodes/Supercapacitors

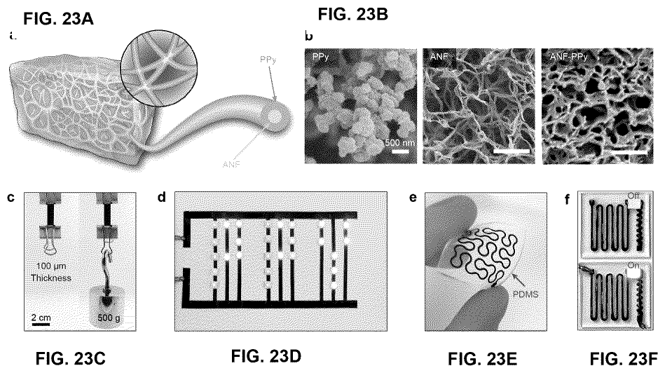

An embodiment of the present invention involves creating a novel material system to achieve strong and high-conductivity hydrogels as high-performance electrochemical electrodes for various device applications. Distinct from previous approaches, a nanostructured conductive network is built via conformal assembly of a conducting polymer (polypyrrole (PPy) onto a well-established nanofibrous network (aramid nanofibrous (ANF) hydrogel network) (FIG. 23A). The resulting ANF-PPy hydrogel (ANF-PPy), which possesses unique conductive pathways of hyper-connective topology, has a series of superior properties distinguishable from those of other conductive hydrogels. It involves an ultralow threshold for PPy electrical percolation, superior conductivity, high fracture strength, and great electrochemical stability. Benefiting from the bulky nanoarchitectures and the intrinsic pseudocapacitance of PPy, ANF-PPy has an outstanding specific capacitance in acidic electrolytes, indicating its potential applications as stretchable supercapacitors. Furthermore, ANF-PPy prepared readily from one-step-synthesis allows the printing of patterned electrodes on soft PDMS substrate, serving as a high-performance platform for bioelectronic applications. The novel strategy to fabricate ANF-PPy nanofibrous conductive hydrogel creates new opportunities for electrode design which can be readily scalable to a wide range of flexible devices.

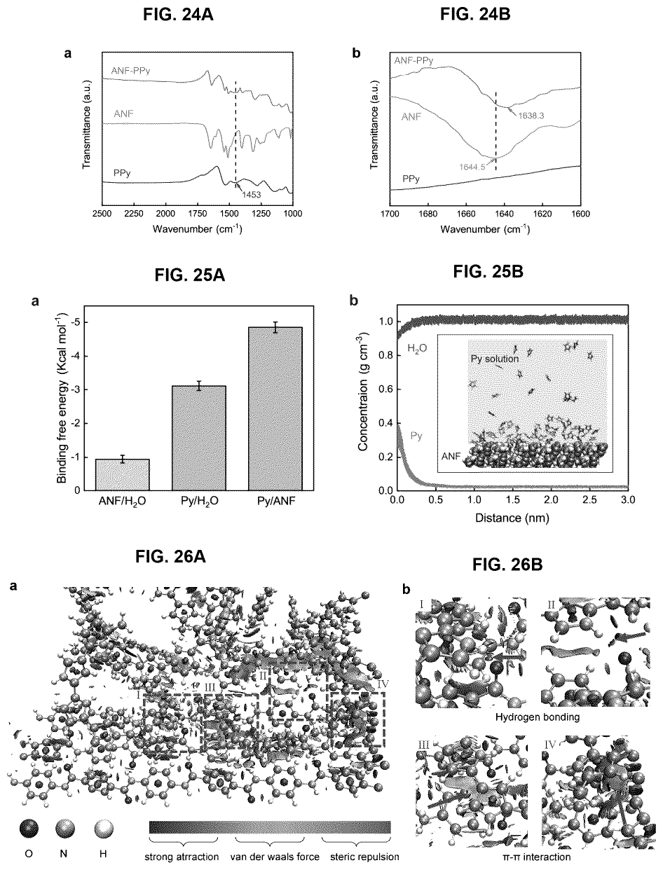

ANF-PPy conductive hydrogel (ANF-PPy) is fabricated by in-situ polymerization of PPy within an ANF hydrogel network. To achieve ANF hydrogels with high solid content, which is essential for enhancing the conductivity of ANF-PPy (FIG. 22C), ANF hydrogel (7 wt % solid content) is obtained by solvent exchange of high-concentration ANF dispersion (5 wt %) with pure water, serving as a nanofibrous template for following PPy growth. Pyrrole monomers permeate the nanofibrous network of ANF hydrogels, and then undergo chemical oxidative polymerization initiated by ferric chloride (FeCl₃) as an oxidant. The constituents of the resulting composite hydrogel were validated by Fourier transform infrared spectroscopy (FTIR), indicating the successful polymerization process (FIGS. 24A & B). Nanoparticle morphology is observed in PPy polymerized in water solution in the absence of an ANF template (FIG. 23B). Notably, a highly connective network of PPy, in contrast, is generated based on ANF templates. For further understanding the underlying assembling mechanism, molecular dynamics (MD) simulations were conducted to research the ternary system of pyrrole, H₂O, and ANFs (FIGS. 25A & B). The binding free energy between pyrrole and ANF surpasses that of other molecules, resulting in a localized high-pyrrole-concentration area near ANF surface. Accelerated polymerization process occurs in this localized area, initiating PPy deposition on ANFs from the perspective of chemical kinetics. Additionally, MD simulations with non-covalent interaction method according to the reduced density gradient function [52] were performed to research the non-covalent intermolecular interactions of ANF-PPy composite (FIGS. 26A & B). Extensive non-covalent interactions of hydrogen bonding and π-π interactions exist between PPy and ANFs, which also supports the conformal assembling of PPy according to the thermodynamics.

ANF-PPy conductive hydrogel has an all-polymer nanofibrous network which leads to its superior mechanical and electrical properties. High strength and flexibility of the hydrogel are illustrated by a slice of ANF-PPy (7 mm width; 0.1 mm thickness) which can endure a weight loading of 500 g (FIG. 23C) and bear substantial bending and twisting without structural damage (FIGS. 9C & D), respectively. Furthermore, benefiting from its facile fabrication process, various patterning techniques are utilized to pattern ANF-PPy for circuit leads or electrodes. Machining with laser cutting enables ANF-PPy interconnects possible for a circuit with integrated light-emitting diode (LED) lights (FIG. 23D). Additionally, ANF-PPy conductive patterns printed on soft PDMS are achieved by inserting ANF-PPy into PDMS microchannels demonstrating its potential applications for flexible electronics (FIGS. 23E & F).

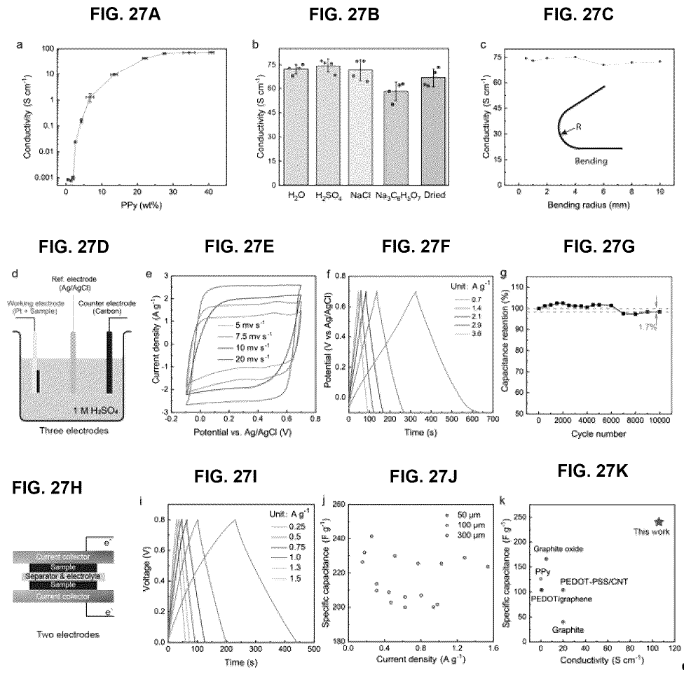

The effect of PPy content on the conductivity of ANF-PPy was analyzed. (FIG. 27A). As PPy is the only conductive component, it can be conceived that too low PPy content (≤1.4 wt %) results in composite hydrogel without electron-transport abilities. However, there is a sharp increase in the conductivity when PPy content reaches a specific threshold, indicating PPy percolates in the 3D network. Notably, the percolation threshold for PPy in the composite is exceptionally low at ˜2 wt %, much lower than the value (˜20 vol %) for nanoparticle electroactive fillers in conductive materials [53,54]. This feature benefits from the unique conductive pathways guided by ANF nanofibrous templates, allowing the engineering of conductive composites with minimal dosage of electroactive fillers. With further increase in the PPy content to ˜42.1%, the conductivity of ANF-PPy (˜46.3% solid content; 100 m thickness) reaches an exceptionally high value of ˜72 S cm-1, outperforming those of other PPy-based conductive hydrogels by several orders of magnitude [55,56].

The electrical stability of ANF-PPy (46.3% solid content; 100 m thickness) under various chemical environments and large-scale mechanical deformation is critical for its role as an electrode material. For instance, supercapacitor electrodes generally work in acidic or salt-rich solutions [57], while electrodes for soft electronics tolerate substantial mechanical deformation in practical applications[58]. The chemical inertness of ANF-PPy was characterized by soaking ANF-PPy in H2SO4 or saline solution of NaCl and sodium citrate at a high concentration of 1 M, respectively. FIG. 27B shows an SEM image with 2.5% ANF at 5 μm and FIG. 27C shows 5% PVA at 1 μm. ANF-PPy treated with H2SO4 and NaCl solution has a minimal change on the conductivity. ANF-PPy in the sodium citrate solution experiences a slight decrease in the conductivity but still keeps a high value at ˜58 S cm-1, possibly due to the reducibility of sodium citrate which may interfere with doping state of PPy [59]. Furthermore, the conductivity of ANF-PPy after removal of solvent by critical point drying is ˜67 S cm-1, indicating its applicability at dry conditions. On the other hand, the electrical stability of ANF-PPy under deformation was determined by its conductivity as a function of the bending radius. ANF-PPy shows negligible changes in the conductivity even at an extreme condition with a bending radius of 0.5 mm, indicating its superior flexibility for soft-device applications.

For the proof-of-concept as electrochemical electrodes, the performance of ANF-PPy as supercapacitor electrodes in both three-electrode and two-electrode configurations were reviewed. For a three-electrode configuration, ANF-PPy on a Pt electrode was used as the working electrode, with a counter electrode of carbon and a reference electrode of Ag/AgCl (FIG. 27D). Initial information about the capacitance of ANF-PPy electrode was provided by cyclic voltammetry (CV) curves (FIG. 27E). At a low scan rate of 5 and 7.5 my s-1, the gravimetric capacitances are 213.8 and 199.3 F g-1, respectively. ANF-PPy shows a typical scan-rate dependence with gravimetric capacitances of 151.3 and 106.6 F g-1 at scan rates of 10 and 20 my s-1, respectively, which is partially due to the incomplete developing of Faradaic capacitance at high scan rates [60]. More definitive information about the capacitance was developed by galvanostatic charge/discharge (GCD) curves (FIG. 27F). The gravimetric capacitance stayed consistent between 200.3 and 202.3 F g-1 at varying current density from 0.7 to 3.6 A g-1. Both methods demonstrate that the ANF-PPy electrode has an excellent capacitance over 200 F g-1. Furthermore, the capacitance of ANF-PPy only has a 1.7% drop after 10,000 charging/discharging cycles at a high scan rate of 80 my s-1, demonstrating remarkable stability in comparison with other supercapacitors[13,25] (FIG. 27G).

As the best-practice method for determining the capacitance of electrodes, a two-electrode symmetrical supercapacitor cell based on two ANF-PPy plates separated by a glass paper permeated with H₂SO₄ electrolyte (1 M) is used to determine the capacitance according to GCD cycles (FIG. 27H). The specific capacitances normalized with area, volume, and gravity are all measured for ANF-PPy electrodes with different thickness of about 50, 100, and 300 m respectively (FIG. 27J, FIG. 4A and FIG. 4B). The areal capacitance increases with the thickness of electrodes, because more electroactive material of PPy is involved in thicker electrodes. As a vital parameter signifying the performance of a supercapacitor, the areal capacitance of ANF-PPy reaches 1040.5 mF cm-2 at a thickness of 300 m, outperforming most of recent supercapacitor electrodes (Table 2). In contrast, the volumetric capacitance decreases with the thickness of electrodes, which is caused by the inhomogeneous distribution of PPy within hydrogel network (i.e., the PPy contents of ANF-PPy decreases with the thickness) (Table 2). Notably, the gravimetric capacitance has a narrow distribution between 200 to 240 F g-1 with different electrode thickness. This consistency of gravimetric capacitance suggests a similar contribution capability of PPy to the total capacitance regardless of the PPy concentrations, indicating efficient conductive pathways built by the unique assembly of PPy. Furthermore, the comparable capacitance values measured under both three-electrode and two-electrode configurations provides support for the robustness of the test methods. Overall, ANF-PPy electrodes show superior properties on both conductivity and capacitance, compared with other supercapacitor electrodes (FIG. 27K and Table 2). Specifically, the conductivity of ANF-PPy exceeds that of other PPy-based hydrogels by several orders of magnitude, benefiting from its unique nanoarchitectures. Additionally, its gravimetric capacitance is also much higher than that of other PPy-based electrodes, and even surpasses that of graphite-based electrodes which has significantly lower density. Its superiority is attributed to its structural integrity and hyper-connective nanofibrous structures.

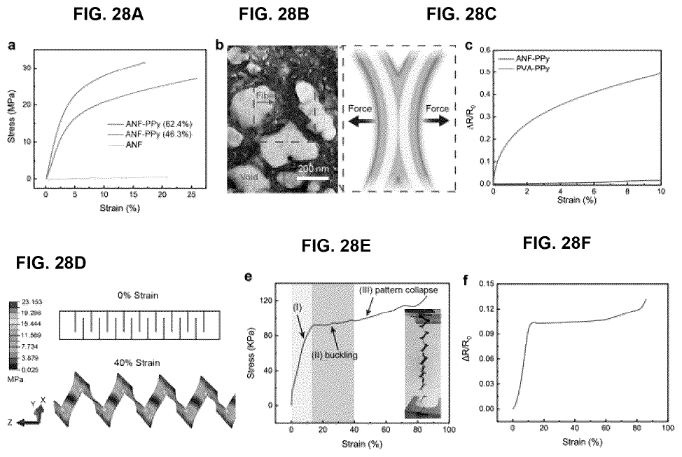

A composition with PPy leads to a marked enhancement of the mechanical properties for ANF hydrogels (FIG. 28A). Pure ANF hydrogel (7% solid content; 100 m thickness) has a favorable strength of ˜0.9 MPa and a tensile Young's modulus of ˜4.8 MPa at a high water content. Considering the strong single ANF of both strength and Young's modulus at GPa levels [62], the mechanical properties of ANF hydrogels are compromised, which may arise from the weak interactions between nanofibers and, thus, easy relative sliding and separation of nanofibers under imposed deformation [63]. Notably, the incorporated PPy bridges the neighboring nanofibers to facilitate load transfer within the network, as visualized by TEM images of ANF-PPy (FIG. 28B). The establishment and reinforcement of binding nodes between nanofibers have been proved to significantly enhance the mechanical performance of nanofibrous network. [64,65] As a result, the ANF-PPy hydrogel (˜46.3% solid content; 100 m thickness) with incorporated PPy at ˜42.1 wt % content shows a superior strength and Young's modulus at ˜27.2 and ˜512.3 MPa, respectively, along with a ductility at ˜22.4%. Lowering the thickness of ANF film facilitates mass diffusion within hydrogel network during the PPy polymerization process, leading to an ANF-PPy composite (˜62.4% solid content; 50 m thickness) with high PPy content at ˜59.3%. This ANF-PPy composite has a high conductivity of ˜106 S cm-1 and an excellent strength and Young's modulus at ˜31.8 and ˜716.3 MPa respectively (Table 2).

Resistance stability under applied deformation is a favorable attribute for conductive materials in many scenarios of device applications [66]. ANF-PPy (˜46.3% solid content; 100 μm thickness) with a high conductivity of 72 S cm-1 only has a negligible resistance change upon 10% tensile strain, showing a gauge factor calculated to be as low as 0.16 (FIG. 28C). The strain-invariant electrical properties of ANF-PPy originates from nanofiber alignment during the deformation, which preserves the intrinsic topology of the conductive pathways [50]. For comparison, a control sample is employed in the form of a conventional conductive hydrogel termed “PVA-PPy.” Unlike the nanofibrous template, this hydrogel is generated through PPy polymerized within PVA hydrogel network. In this case, PPy nanoparticles are randomly distributed within PVA network, leading to a low conductivity of 0.08 S cm-1 of PVA-PPy hydrogel. PVA-PPy hydrogel has a marked increase of ˜50% in the resistance under 10% tensile strain, due to the re-arrangement of the conductive pathway where the interparticle distance of PPy for the electron tunnelling increases in the tensile direction [54,67].

Alternatively, compliant ANF-PPy can be achieved by introducing periodic laser-cut slits (0.75 cm) into a pristine sample (1 cm×5 cm) to increase the ductility with reduced Young's modulus (FIG. 28D). Theoretical simulation by finite element modeling (FEM) reveals the kirigami ANF-PPy has a peak stress of 23 MPa at the tip of crack, below the strength of ANF-PPy, at a tensile strain of 40%, indicating the structural integrity under such deformation. Experimental tensile tests were conducted on kirigami ANF-PPy (7% solid content; 100 μm thickness), and the strain-stress curve shows 3 main regions: (I) The initial elastic region (green) generally caused by crack opening within the strain below 13%. (II) The out-of-plane buckling region (purple) after the applied stress exceeds a critical value. The stress reaches a plateau in the buckling process (13-40% strain). (III) The final pattern-collapse region where cracks propagate due to overhigh stress at crack tips (FIG. 28E). Overall, the kirigami ANF-PPy shows a markedly reduced Young's modulus at KPa levels and can sustain tensile deformation above 80% before failure, which expands its applications in soft electronics [68]. Furthermore, the resistance change of kirigami ANF-PPy remains at a low level (<12%) during tension before breakage. An increase in the resistance is observed at the beginning and end of tension, resulting from the crack opening [69] and propagation, respectively.

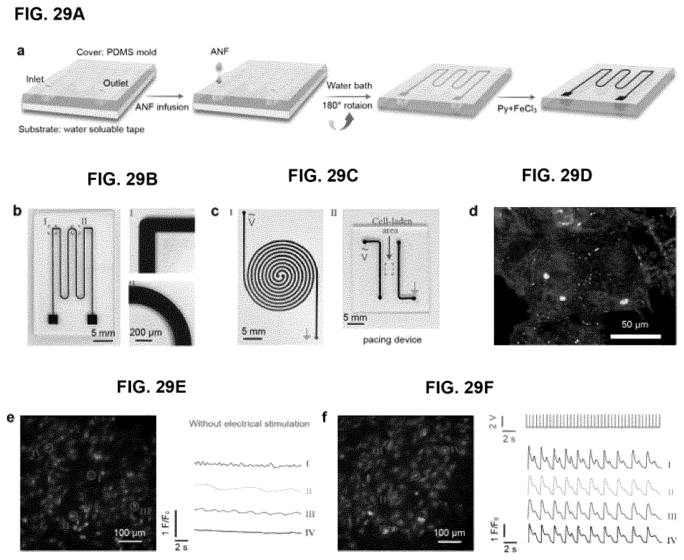

The favorable bioelectronic interface created by ANF-PPy is initially evidenced by its low impedance in phosphate-buffered saline (PBS) (FIG. 4G). Compared with an Au foil of the same thickness, ANF-PPy (7% solid content; 100 m thickness) has markedly lower areal impedance under frequency below 1000 Hz, which covers the frequency band for common bio-signals [70]. The low impedance allows ANF-PPy to be used as a favorable bioelectrode to record physiological signals, such as electrocardiograph (ECG) signals, with high quality (FIG. 21A). Alternatively, the advantages of ANF-PPy bioelectrodes are also revealed by its capability in delivering electrical stimulation. The bulky nanostructures of ANF-PPy provide high interfacial capacitance, allowing good control over the current flow with high charge storage capability and charge injection capacity (FIGS. 4D and 4F). Furthermore, due to the capacitive nature, ANF-PPy electrodes can avoid non-reversible Faradaic reactions, which may bring on electrode degradation and harmful byproducts during electrical stimulation [71]. To pattern ANF-PPy for custom-built bioelectrodes, a PDMS mold containing microchannels was prepared by microfabrication techniques. The PDMS mold was placed face down on a water-soluble tape, and ANF dispersion (3 wt %) was infused from the inlet into the microchannel (FIG. 29A). Patterned ANF hydrogel was generated by soaking the PDMS in water for both the removal of the tape and solidification of ANF dispersion. After that, a final ANF-PPy pattern embedded within the PDMS channel was achieved through the incorporation of PPy into the hydrogel network by sequential treatment with Py and FeCl₃ solutions. The successful printing of ANF-PPy on PDMS was demonstrated by a micropatterned ANF-PPy showing high printing resolution of 200 μm linewidth and high printing fidelity with clear right-angle and curve corners (FIG. 29B).

To confirm the performance of ANF-PPy bioelectrodes in delivering electrical stimulation, two parallel ANF-PPy electrodes were printed on PDMS (FIG. 29C). First, electrogenic cells of cardiomyocytes (CMs) seeded on the surface of ANF and ANF-PPy hydrogels show great cell viability, while ANF-PPy, in comparison with ANF, provides more biocompatible substrate for CM attachment (FIGS. 5D and 5E). This result may originate from the protein-affinity surface of ANF-PPy with appropriate hydrophilicity [72] (FIGS. 22A and 22D). The favorable biointerface between the ANF-PPy electrodes and cells is further validated by fast maturation of CMs cultured on ANF-PPy, as evidenced by pronounced expression of typical cardiac markers of troponin T and connexin-43 for CMs after culture of 5 days (FIG. 29D). A typical cardiac pacer device to deliver electrical stimulation to CMs was connected to two parallel linear electrodes with cells laden in the area between two electrodes. [73,74] Two linear ANF-PPy electrodes (0.3 mm thickness, 0.6 mm width, and 20 mm length) were printed on PDMS with the gap width between two parallel electrodes of 5 mm (FIG. 29C (II)). Due to the capacitive nature of electrodes, bioelectrical stimulations could be delivered through charging and discharging the electrodes to avoid irreversible Faradaic reactions at the biointerface. Previous research has revealed that long-term electrical stimulation by pulsatile electrical fields can accelerate the maturation of CMs and generate synchronously contractile cardiac constructs [71]. To investigate CM responses upon electrical stimulation at the initial period, CMs were seeded on the pacing device between two electrodes. Three days after CM seeding without electrical stimulation, CMs showed weak and random excitation by monitoring cellular electrical activity through calcium imaging (FIG. 29E). However, after electrical signals of monophasic pulses (2 Hz) were input through ANF-PPy electrodes for 10 min, overdrive pacing of these immature CMs was achieved, but the contraction rate synchronized to the frequency of 1 Hz, half the value of the pacing frequency (FIG. 29F). As shown from the plots of calcium transients, after each synchronized calcium spike, immature CMs take a long time, about 2 s, to reduce the calcium concentration to the previous low level. Between two calcium spikes, a much weaker excitation is observed at high intracellular calcium concentrations, which is compulsorily driven by the external electrical stimulation. This compulsory excitation at high intracellular calcium concentrations may enhance CMs' capability in calcium ion transport and contribute to their synchronized contraction at high frequencies during long-term electrical stimulation.

Thus, a high-performance conductive hydrogel has been developed through nano-structuring PPy conductive pathways with topology copied from the hyper-connective nanofibrous network of aramid hydrogels. Efficient electron/ions transport capability arising from the nanoarchitectures allows ANF-PPy superior electrical conductivity and capacitance. ANF-PPy also shows excellent mechanical properties and electrical stability even under extreme physicochemical conditions. Enabled by these merits, ANF-PVA is utilized as electrodes for supercapacitors with favorable specific capacitance. The simple fabrication processes enable ANF-PPy ready integrability into patterning techniques with other materials, generating patterned ANF-PPy suitable for efficient bioelectrodes used in physiological-signal recording and delivering. This invention not only presents a nanostructured conductive hydrogel addressing the existing limits of conventional conductive hydrogels, but also provides an efficient methodology for a new fabrication strategy of hydrogel electrodes applied in flexible devices and bioelectronics.

Molecular dynamics (MD) simulations were conducted with GROMACS 2022 under the General AMBER Force Field (GAFF2) force field. [157,158] Initially, the structures of H₂O, Py, and ANF molecules were constructed and optimized by Gaussian 16, and then the advanced restrained electrostatic potential charges (RESP2) of these molecules were assessed by Multiwfn 3.7 for following simulations. [159,140]. According to the experimental conditions, a cuboid periodic boundary condition box [5] (5×5×8 nm3) with a H₂O-to-Py molar ratio of 90:1 was generated by random distribution. Three stacked infinite layers of ANF molecules were orderly organized though intermolecular hydrogen bonding, which was placed on the bottom of the box to act as the ANF surface. This system was further optimized by energy minimizations using 5,000 steepest descent steps to exclude unfavorable organizations. After that, an equilibrium state of the system was achieved in an isothermal-isobaric ensemble at 298° K and 1 atm. Finally, a 50 ns production run was conducted, and data of the last 10 ns were collected for further analysis. The real-space cut-off for noncovalent interactions is 12 Å and the time step is 1 fs [162]. Long-range electrostatic interactions and van der Waals forces were calculated using the particle mesh Ewald method. The visualization process was assisted by VMD software, [162].

To probe the non-covalent interactions between ANF and PPy, a cuboid periodic boundary condition box (5×5×8 nm3) with a bottom layer of ANF and 45 PPy molecules [159](degree of polymerization: 10) was generated. Similar to the aforementioned methods, a balanced organization between ANF and PPy was achieved after 50 ns MD simulation in an isothermal-isobaric ensemble at 298° K and 1 atm. Molecular interactions were further studied by a non-covalent interaction method with Multiwfn 3.7 according to a reduced density gradient (RDG) function. [163].

$$RDG ( r ) = \frac{1}{2 {( 3 {π}^{2} )}^{1 / 3}} \frac{\vert ∇ p ( r ) \vert}{{[ ρ ( r ) ]}^{4 / 3}}$$

where ρ is electron density and r is coordinate vector. Visualization process was assisted by VMD software with the RDG isosurfaces of a cut-off value at 0.5 a.u. sign(λ₂)ρ is mapped on the isosurfaces between −0.035 a.u. (blue) to 0.02 a.u. (red).

A denser nanofibrous network leads to a higher conductivity of fabricated ANF-PPy hydrogels for solid contents of ANF hydrogels between 1.4% and 7%. ANF hydrogels with solid contents of 1.4%, 2.7%, and 7% were fabricated by water exchange of ANF dispersion of 1%, 2%, and 5% solid contents, respectively. See FIG. 22C.

Chemical compositions characterized by FTIR spectroscopy are shown in FIG. 24A for ANF, PPy, and ANF-PPy. ANF-PPy has a peak at 1453 cm-1 corresponding to the characteristic peak of ═C—N asymmetric ring stretching for PPy, demonstrating the successful incorporation of PPy. FIG. 24B shows magnified plots of the spectra of aramid C═O stretching vibration. ANF-PPy has a slight red shift of C═O stretching vibration compared with ANF, indicating the effects of hydrogen bonding between ANF and PPy. FIG. 25A shows molecular interactions of ANF hydrogel in Py solution for binding free energy between ANF, H₂O, and Py molecules. FIG. 25B shows the distribution of H2O and Py molecules in a localized area near ANF surface.

Noncovalent interactions between ANF and PPy can be sorted among strong attraction, van der waals force and steric repulsion. FIG. 26A shows this as a reduced density gradient (RDG) function isosurface map of ANF and PPy. FIG. 26B is a magnified display of the corresponding area in RDG function isosurface map, showing the existence of hydrogen boding and π-π interaction between ANF and PPy.

The parameters of ANF and ANF-PPy hydrogel firms are listed in Table 3

TABLE 3
Parameters of ANF and ANF—PPy hydrogel films

| | PPy | Solid | | | Young's | | |
|---|---|---|---|---|---|---|---|
| | content | content | Thickness | Conductivity | modulus | Strength | Elongation |
| | (%) | (%) | (μm) | (S cm⁻¹) | (MPa) | (MPa) | (%) |
| ANF | 0 | 7 ± 0.3 | 100 | — | 4.8 ± 0.14 | 0.9 ± 0.03 | 21.7 ± 0.92 |
| ANF—PPy | 59.3 ± 3.9 | 62.4 ± 2.7 | 50 | 105.9 ± 5.8 | 716.3 ± 39.4 | 31.8 ± 2 | 16.8 ± 1.1 |
| ANF—PPy | 42.1 ± 2.7 | 46.3 ± 1.5 | 100 | 72 ± 3.2 | 512.3 ± 20 | 27.2 ± 1.7 | 22.4 ± 2.6 |
| ANF—PPy | 30.8 ± 3.2 | 35.8 ± 1.7 | 300 | 43.4 ± 2.7 | 349 ± 25 | 21.8 ± 1.6 | 21.3 ± 2.3 |

### Solar Desalination

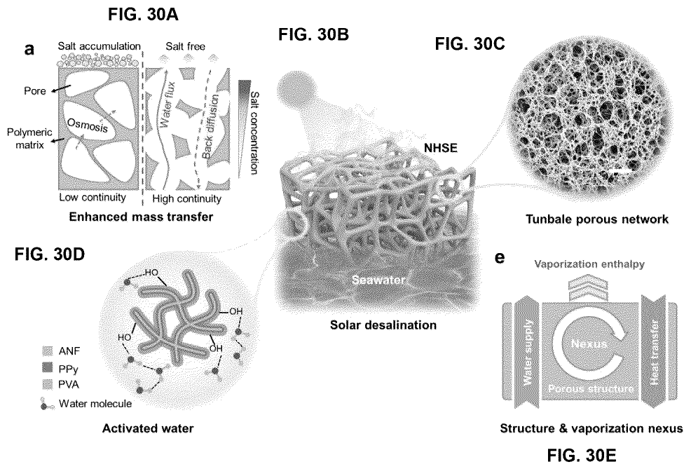

The present inventors recently developed a series of porous composites based on aramid nanofibers (ANFs) with tunable microstructures and compositions, enabling a versatile platform for the engineering of functional hydrogels for solar desalination.[111,112]. It has been determined that nanofibrous hydrogel solar evaporators (NHSEs) can be designed which involve tunable open porous network to achieve high-performance solar desalination (FIG. 30). The self-assembled network provides continuous microchannels for efficient mass transfer, thus preventing salt accumulation. It also presents abundant hydrophilic groups that facilitate water activation for reduced vaporization enthalpy, as well as uniformly loaded polypyrrole (PPy) for effective photothermal conversion. Through quantitative analysis on the interplay between various structural and thermodynamic parameters, in combination with rational experimental tuning, a materials configuration showing stable evaporation rate of 2.85 kgm⁻²h⁻¹ in 20% brine under solar illumination has been identified. The outstanding performance of NHSEs and the underlying design principles may provide insights for the development of advanced solar desalination systems.

The NHSEs were fabricated based on solution processing. Specifically, ANFs and polyvinyl alcohol (PVA) were dispersed in dimethyl sulfoxide (DMSO) followed by mixing in the liquid phase. The incorporation of PVA provides extensive hydrophilic interface for water activation, and it toughens the fibrillar networks via hydrogen bonding with ANFs. [87,111,113] Solid hydrogels with open porous network were obtained by replacing DMSO with water. Tuning of porosity was achieved by varying the ANF and PVA contents in the hydrogels. Samples with high solid content (e.g., ≈12 wt %) demonstrates hyper-connective nanofibrous networks ascribed to the high aspect ratio of ANFs and interfibrillar interactions (FIG. 31A-C).[111,112] On the other hand, with the decrease of solid content (e.g., to ≈3.9 wt %), a hierarchical open porous structure started to form as ANFs aggregate into fiber bundles followed by assembly into a network at a higher length scale (FIGS. 31D, 31E). The phenomenon is possibly induced by the extensive hydrogen bonding between fibrils and the energy minimum under low solid content. SEM images of a bulk sample indicate uniform distribution of ANF in the hydrogel. A dependence of the pore size on the ANF concentration was observed. Interactions between water molecules and the hydrophilic matrix are conducive to the reduction of vaporization enthalpy.[114] The water activation effect of hydrated polymeric network can be verified by Raman spectrum. An ultralow vaporization enthalpy of 1050 J g⁻¹ was achieved by NHSEs

The resulting 3D networks exhibit pore sizes varying by ≈10 times (i.e., from 28 to 2491 nm) within a relatively narrow range of solid content (≈3.9%-10.9%), allowing for convenient tuning of transport properties. The high porosity (>90%) of the nanofiber hydrogels (FIGS. 9C and 9D Figure S5, Supporting Information) is conducive to the mass transfer as indicated by the equation of effective diffusion coefficient (Deff) in hydrogels: [115 40]

$${D}_{eff} = \frac{{D}_{0}}{1 + p ( 1 - ϵ )} \qquad ( 1 )$$

where D0 represents the bulk solution diffusion coefficient, p is the structure factor, and ϵ is the porosity. In addition, the hyperconnective nanofiber network endows high structural stability and mechanical strength to the hydrogels (FIG. 31G). The NHSEs were obtained by incorporating PPy as photothermal conversion material with ANF-PVA network. Notably, the PPy polymerizes along the nanofibers without compromising the porous structure of NHSE, which is partly due to the π-π stacking and hydrogen bonding between ANFs and PPy.[112] Due to the simple solution-based processing, NHSEs can be molded into various 3D shapes (FIG. 31H), allowing for flexible designs for evaporators.

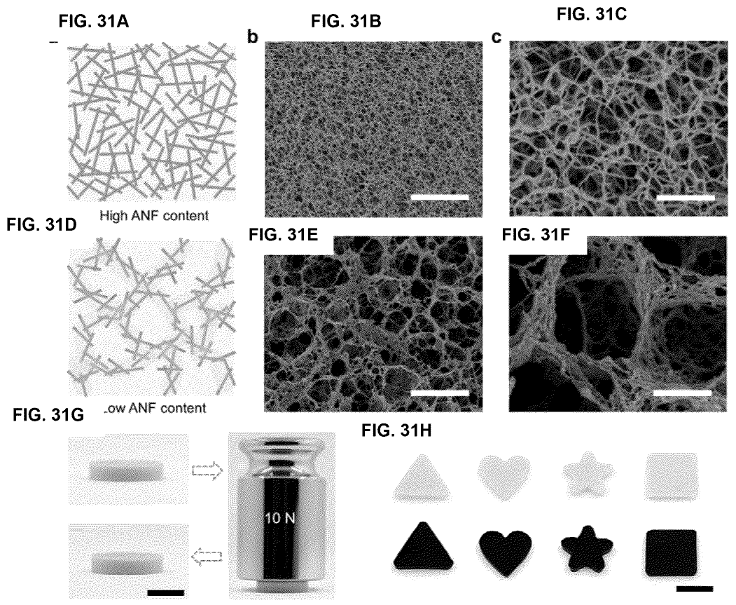

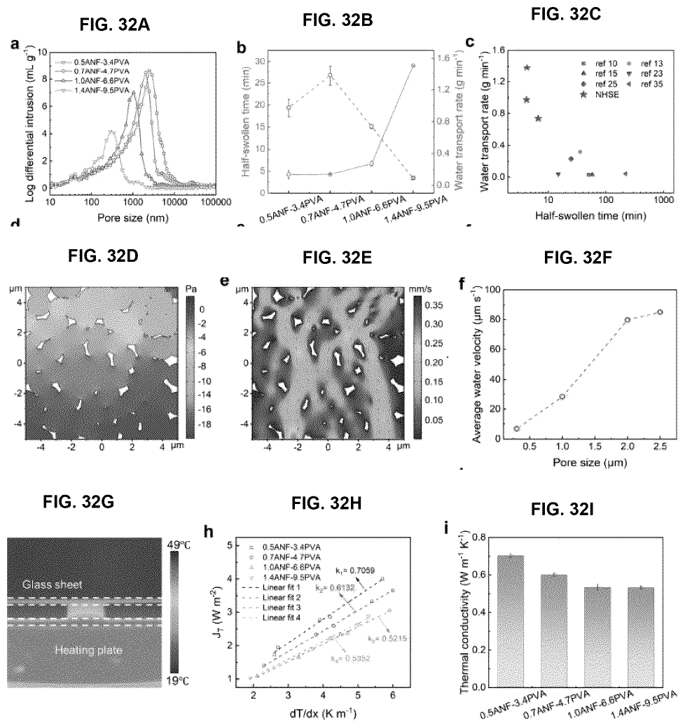

Swelling experiments demonstrate excellent water transport properties of NHSEs. The swelling capacity of hydrogels can be obtained by measuring the water content (Q):

$$Q = ( W - {W}_{d} ) / {W}_{d} \qquad ( 2 )$$

where Wd and W represent the weight of an aerogel sample prepared by critical point drying and the weight of corresponding hydrogel after hydration in water, respectively. The NHSEs present tunable saturated water content (Qs) ranging from 5.24 to 11.85 g g-1 Water transport rate V can be estimated by an established formula: [85]

$$V = \frac{0.5 {Q}_{s}}{t} \qquad ( 3 )$$

where t represents the half-swollen time. The water transport rate increases with increasing pore size, partly due to the lower tortuosity in hydrogel with larger pores (FIG. 32B). A sharp increase of water transport rate occurred at hydrogel with 1.0% ANF and 6.6% PVA, which is ascribed to the formation of hierarchical structures and larger pores. The highest water transport rate of 1.38 g min was achieved by the sample with 0.7% ANF and 4.7% PVA (FIG. 32B). As the ANF content drops below 1%, the ANF-PVA hydrogels demonstrated fast water transport superior to most of the reported HSEs (pore size≈100 m) for solar desalination (FIG. 32C). With a similar pore size, the ANF-PVA hydrogels exhibit water absorption rate two orders of magnitude higher than that of PVA hydrogels, demonstrating the advantages of open microchannels in NHSE.

The effect of pore size on the water transport velocity was confirmed by numerical simulation. A computational model composed of overlapping circles was created in COMSOL to simulate the interconnected pores in NHSEs, where the diameter of the circle corresponds to the average pore size of the hydrogel. One sun illumination was applied at the upper boundary of the model for evaporation. The negative pressure on surface generated by evaporation combined with capillarity drives the water transport in microchannels (FIGS. 32D & E). The simulated water velocity in the microchannels increases as the pore size increases, which is consistent with the result obtained in the swelling experiments (FIGS. 32B & F).

The NHSEs also exhibit low thermal conductivity, which facilitates heat localization during solar desalination. Considering that the NHSEs works in a hydrated state, measurement of thermal conductivity was conducted using hydrogels with saturated water content. The thermal conductivity of NHSEs decreases with decreasing pore size (FIGS. 32G & H), which is attributed to the increased thermal resistance by more interfaces and boundary effects with smaller pores. An ultralow thermal conductivity of 0.53 W m⁻¹ K⁻¹ is achieved by hydrogel with 1% ANF and 7.4% PVA. (FIG. 32I). The PPy in NHSEs induces negligible deterioration on water transport and heat preservation.

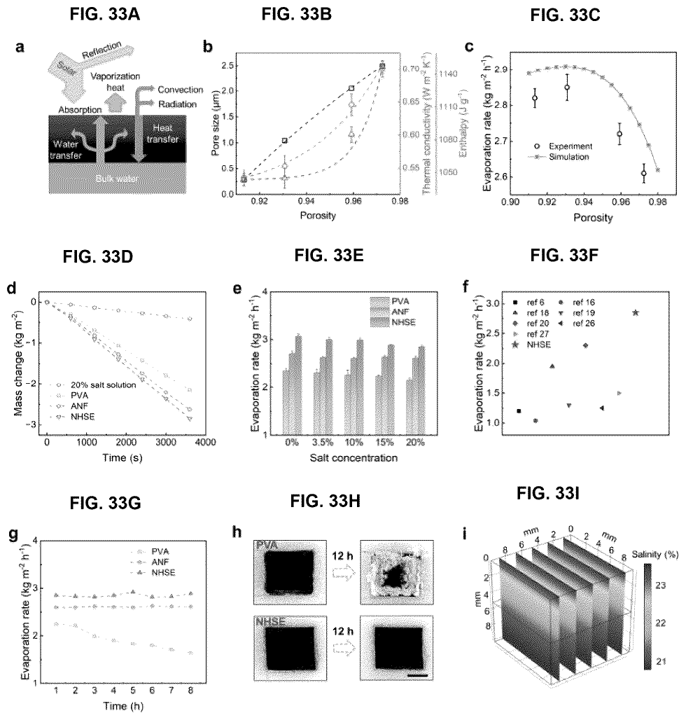

Indeed, there is usually a trade-off between efficient water transport and heat localization for effective photothermal evaporation. Furthermore, the variation of evaporation enthalpy originating from materials and microstructures may also present significant influences. Therefore, it is critically important to understand the quantitative contributions of various structural, thermodynamic, and kinetic parameters to the evaporation performance for the engineering of NHSEs. To clarify the complex nexus between the physical processes, a computational model was developed based on the energy balance during solar evaporation (FIG. 33A). The model was conducted by COMSOL Multiphysics 5.6, which coupled the heat transfer in porous media and the porous media flow into a stationary solver. All NHSEs were assumed with the same thermal convection and radiation to the environment (Equations (13) and (14)). Thermal insulation assumption was adopted as the boundary conditions of four side faces due to the wrapped thermal insulation foam in the experiments. The NHSEs demonstrated excellent solar absorptance above 98% as indicated by UV-vis-NIR spectra, which was set as energy input efficiency of the evaporation system. Models of NHSEs were constructed based on the various porosity, pore size, vaporization enthalpy, and thermal conductivity. Quantitative relationships between the various parameters and porosity of NHSEs were fitted based on experimental data (FIG. 33B, Equations (17)-(19)), which facilitate the calculation of evaporation rate as a function of porosity of NHSEs.

The simulation shows a maximum evaporation rate achieved by NHSEs with an intermediate porosity of 93%, which is consistent with the experimental observation (FIG. 33C). These results can be understood from the coupling between vaporization enthalpy, water transfer, and heat localization of NHSEs during evaporation. On the one hand, NHSEs with lower porosity exhibit lower vaporization enthalpy and thermal conductivity, which facilitates evaporation. On the other hand, their smaller pores lead to less efficient water transfer, which might downgrade the evaporation performance. Indeed, the intermediate porosity associated with the maximum evaporation rate represents an optimum regarding these correlated processes. Simulations with independently controlled variables indicate further design principles for NHSEs. Specifically, the evaporation performance is tightly correlated with the porosity and vaporization enthalpy, which represent water transport and energy efficiency, respectively. However, the effect of thermal conductivity on the evaporation rate is relatively minor especially with pore sizes below 30 μm. This result may be related to the limited fluid flow in small pores regardless of the differences in temperature field induced by various thermal conductivities. It should be noted that the present simulation is based on the constitutive relationships derived from macroscopic models, which may have limitations when investigating complex thermal hydraulic behaviors of mesoscopic porous media. Further development of microscopic models and examinations with other materials systems would be helpful for a complete mechanistic understanding.

The NHSE with 1% ANF and 6.6% PVA exhibited the best evaporation performance, which was utilized for the application of solar-driven desalination. Stable water transfer and thermal conductivity of NHSEs in various salt solutions indicate robust performances for treating brines. Evaporation performance of NHSEs was measured under one sun illumination using a custom testing device. Compared with the pure ANF hydrogels and PVA hydrogels, NHSEs achieved evaporation rates 10%-30% higher (FIGS. 33C & 33D, which results from the combination of porous nanofiber network and hydrated polymer matrix. NHSEs demonstrate the lowest surface temperature among these evaporators due to the significant vaporization that takes away heat from the surface. [87] This low surface temperature facilitates the heat localization by minimizing the heat radiation to the environment. With brines with salinity ranging from 0% to 20%, the NHSE achieved high evaporation rate from 3.1 to 2.85 kg m-2 h-1 (FIG. 33D and efficiency from 90% to 83%, which outperforms most of the reported works for strong brine desalination (FIG. 33F).

The open porous network of NHSEs enables efficient back diffusion of salt from evaporation surface to the bulk water, resulting in excellent salt-resistance. The desalination stabilities of various hydrogel evaporators were evaluated by conducting continuous evaporation in 20% brine (FIG. 33F). Both NHSE and ANF hydrogels demonstrated stable evaporation, verifying the efficient mass transfer in 3D nanofibrous network. In contrast, a decline of evaporation rate was observed in PVA hydrogels after 2 h of evaporation. Severe salt accumulation occurred at the surface of PVA hydrogel after 12 h of continuous evaporation in 20% brine, while no salt crystals appeared on NHSE (FIG. 33H). Additionally, the PVA hydrogel evaporator suffered a dramatic shrinkage at the evaporating interface during the solar desalination, prompted by the mismatch between water supply and vaporization. Continuous evaporation concentrates the salt solution in the water channels, which may lead to deswelling of polymeric matrix driven by osmotic pressure. This phenomenon is represented by the shrinkage of PVA hydrogel in 20% brine. The shrinkage further decreases the water transfer in the hydrogel, resulting in a vicious cycle. Inversely, the ANF network in NHSEs provides a rigid skeleton that prevents the shrinking effect and maintains the efficient water transfer in high-salinity brine during solar desalination. The efficient mass transfer enables self generation of NHSE by dissolving salt crystals added on the surface. Moreover, numerical simulations indicated that the steady-state salt concentration at the evaporation surface of NHSE stays below the saturation level (FIG. 33I), which prevents salt crystallization. This property of NHSEs is beneficial for long-lasting solar desalination with strong brine.

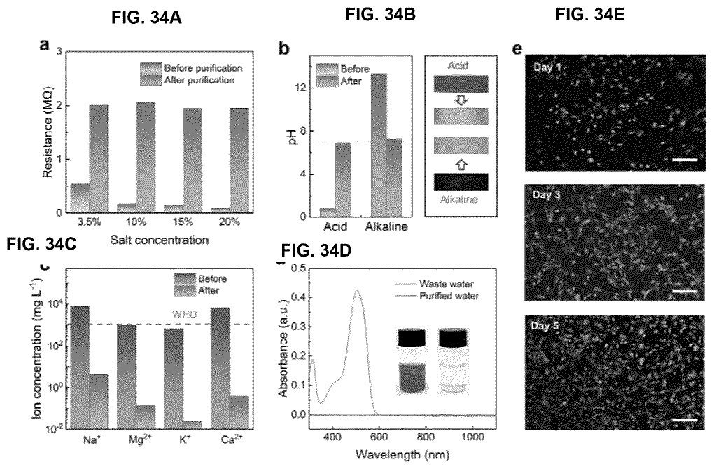

The quality of water treated by NHSEs can be further characterized. The decrease of salinity is validated by the increased resistance of the water samples from kilohm to megohm (FIG. 34A) via a multimeter with the fixed distance between two electrodes. In addition to brine, NHSE can be used to purify acid and alkaline liquids. The purified water samples demonstrate pH values close to neutral (FIG. 34B). Moreover, solar desalination was performed using real seawater collected from Kennedy Town, Hong Kong. The concentrations of four typical elements (Na+, Mg2+, K+, Ca2+) in seawater decreased by approximately three orders of magnitude after purification, which meet the standards for drinking water set by the World Health Organization (FIG. 34C). Liquid samples with colored containments were also purified, leading to colorless and transparent water as proved via the UV-vis spectra (FIG. 34D). Furthermore, successful cell proliferation in the nutrient solution with the purified water as the solvent indicates the desired water purity (FIG. 34E). An efficient water production of 10.4 kg m-2 d-1 was achieved by NHSE in an outdoor evaporation experiment, indicating the reliability of NHSE for practical applications.

For this purpose, i.e., efficient solar desalination, the ANF-PVA hydrogels were prepared using solvent exchange methods reported by previous work. [111 36] Briefly, ANF and PVA dispersion was prepared by dissolving Kevlar para-aramid pulp (Type 979; DuPont) and poly(vinyl alcohol) (PVA; Mn≈75.000; hydrolysis degree of 96%≈98%; Aladdin Reagent) in dimethyl sulfoxide (DMSO; Aladdin Reagent) under magnetic stirring at 95° C. for 7 days, respectively. The ANF (1.5%) and PVA (10%) dispersion were mixed up with the ratio of 1:1, followed by poured into a mold. The hydrogels were obtained after exchanging DMSO with deionized (DI) water completely. Solid content of the hydrogel was regulated by diluting the ANF/PVA mixture with DMSO or evaporating excess DMSO in vacuum oven. No volume change occurred in all NHSEs after solvent exchange. PVA hydrogels were prepared using freeze-thaw method. Typically, 0.5 mg mL⁻¹ of PVA aqueous solution was poured into a mold then frozen in the refrigerator at −24° C. Thereafter, the frozen mixture was thawed at room temperature. PVA hydrogels were obtained after five freeze-thaw cycles.

For the coating of PPy, the hydrogels were soaked with 0.5 m FeCl₃ (Sigma-Aldrich) solution. Then the hydrogels were exposed to pyrrole (Sigma-Aldrich) vapor at 4° C. for 10 min. After polymerization, the hydrogels were washed with DI water to remove unreacted ions and pyrrole.

SEM images were obtained by scanning electron microscope (Hitachi S4800 FEG) after dried by critical point dryer (CPD; Tousimis Autosamdri 931). Pore size distribution was characterized by mercury intrusion porosimeter (MIP, AutoPore IV 9500). Absorption spectra of hydrogels and liquids were conducted using UV-vis-NIR spectrometer (UV 36001 plus, Shimadzu) and UV-vis adsorption spectra (UV-2600, Shimadzu), respectively. Ohmic resistance and pH value of water samples were tested by a digital multimeter (Victor, VC890C) and a pH meter (Mettler Toledo, FiveEasy), respectively. Ion concentration was measured by inductively coupled plasma mass spectrometry (ICP-MS, ELAN DRC-e, Perkin Elmer).

The porosity (E) of samples was calculated by:

$$ϵ = ( 1 - \frac{11.5 {ρ}_{0}}{0.5 {ρ}_{a} \to 10 {ρ}_{p}} ) \times 100 \% \qquad ( 4 )$$

where ρ0, ρa, and ρp represent the density of the bulk sample, ANF (1.44 g cm-3), and PVA (1.19 g cm-3), respectively. The coefficients in the equation refer to the ratio of ANF and PVA (1.5:10) in hydrogel.

Solar vapor generation experiments were conducted.: The hydrogel evaporator with size of 1 cm×1 cm×0.5 cm was floated on the water for purification with the assistance of polyethylene foam. A solar simulator (Aulight, CEL-S500-T5) was used to provide continuous one sun illumination. The solar intensity on the evaporator surface was measured using a solar power meter (Tenmars, TM-207). The mass change of the evaporation device was measured using an electronic balance (Mettler Toledo) after stabilization under one sun for 0.5 h. An infrared camera (Fluke Ti480) was used to record the temperature change of the evaporator. All the evaporation tests were conducted at room temperature (25° C.) with the relative humidity of 45%.

The swelling behavior of NHSEs was also studied. The water transport rates of the hydrogels were measured by evaluating their swelling behaviors. A CPD dried hydrogel with the size of 1 cm×1 cm×0.5 cm was immersed in the pure water or salt solutions at 30° C. to monitor the weight change until fully swollen (no weight change in 0.5 h). Specially, the samples were wiped by air-laid paper to remove the water in capillary channels before each measurement.

The thermal conductivities of hydrogels were measured according to Fourier's law. The hydrogel with the size of 1 cm×1 cm×0.3 cm was sandwiched between two 1 mm glass plates with the conductivity of 1.3 W m-1 K-1. The sandwich was heated at different temperatures on a heating plate to record the steady-state temperatures at interfaces (heating plate-bottom glass, bottom glass hydrogel, and hydrogel-top glass) of the sandwich using thermocouples. Assuming that the glass plate and hydrogels have similar heat fluxes, the thermal conductivities of hydrogels were calculated by:[117]

$${J}_{T} = - k \frac{dT}{dk} \qquad ( 5 )$$

where JT is the heat flux per unit area, k is the thermal conductivity of the material, and dT/dx is the temperature gradient at the heat transfer direction.

Equivalent vaporization enthalpies of hydrogels were obtained by measuring the evaporation rates of samples in the dark condition. [87] Water and hydrogels with the same evaporation area were placed in the same dark environment at 25° C. and 45% of humidity for 24 h to ensure the same energy input (Uin). The equivalent vaporization enthalpy was obtained by:

$${U}_{i n} = {h}_{0} {m}_{0} = {h}_{eq} m \qquad ( 6 )$$

where h0 is the enthalpy of pure water (≈2450 J g-1), m0 is the mass loss of water, m is the mass decline of hydrogel, and heq is the equivalent vaporization enthalpy of the hydrogel, respectively.

Evaporation efficiencies are calculated by the equation as below:

$$η = \frac{\dot{m} {h}_{eg}}{{C}_{opt} {q}_{i}} \qquad ( 7 )$$

where η represents the evaporation efficiency, m is the evaporation rate, heq is the equivalent enthalpy, Copt is the optical concentration, and qi is the solar irradiation power.

The commercial software COMSOL Multiphysics v5.6 was used to simulate the water transfer, evaporation flux, and salt distribution of NHSEs.

A model was constructed for simulation analysis of water velocity in microchannels. The interconnected pores were represented by overlapping circles with various diameters corresponding to the average pore sizes of different NHSEs. A constant solar irradiation of 1 kW m-2 was applied to the top surface for driving evaporation. The water flux entered the evaporator equals to the escaped water flux caused by evaporation. Considering the water confinement of the capillary channels, the water transport was simulated by solving the following equations:

$$ρ ( u \times ∇ ) u = ∇ \times ( - p l + μ ( ∇ u + {( ∇ u )}^{⊤} ) ) + F + ρ \text{?} \qquad ( 8 )$$

$$ρ ∇ \times \text{?} = 0 \qquad ( 9 )$$

$$\text{?} \text{indicates text missing or illegible when filed}$$

where the ρ and μ are the mass density and viscosity of water. U, p, and T represent the fluid flow speed, pressure, and temperature of water, respectively. I and F are the second order unit tensor and gravity of water, respectively.

The simulation of solar evaporation considers vaporization enthalpy, heat transfer, and water transport in NHSEs. The 3D domain (1 cm×1 cm×0.5 cm) referred to the size of NHSE was discretized with a mesh consisting of up to 20 000 tetrahedron-dominant elements. The heat transfer in solar evaporator follows the equations as below:

$$ρ {C}_{p , tot} \text{?} \times ∇ T + ∇ \times q = Q + {Q}_{evap} + {Q}_{c} + {Q}_{e} \qquad ( 10 )$$

$$q = - k \text{?} ∇ T \qquad ( 11 )$$

$${Q}_{evap} = - {h}_{evap} \dot{m} \qquad ( 12 )$$

$$Q \text{?} = - h \text{?} ( T - {T}_{e} ) \qquad ( 13 )$$

$$Q \text{?} = - \text{?} ( {T}^{4} - {T}_{e}^{4} ) \qquad ( 14 )$$

$$\text{?} \text{indicates text missing or illegible when filed}$$

where ρ is the density, Cp,tot is the total heat capacity at constant pressure, q is the conductive heat flux, keff is the effective thermal conductivity, u is the velocity field, Qevap is the latent heat source in NHSE, hevap is the vaporization enthalpy, m is the evaporation flux on the evaporating surface, Qc is the heat convection on the surface, he is the convection coefficient (10 W m-2 K-1), Qe is the heat radiation on the surface, Em is the surface emissivity (0.9), σ is the Stefan-Boltzmann constant (5.67×10-8 Wm-2 K-4), Te is the environment temperature (298.15 K), Q is the energy input on the top surface of evaporator, qi is the solar intensity (1 kW m-2) applied to the evaporator, and a is the solar absorptance efficiency (98%) of NHSE.

As the water evaporation comes from the difference between the mass concentration of the evaporating surface and the air, the m can be calculated as below:[118]

$$\dot{m} = - {k}_{m} \frac{M}{R} ( \frac{P \text{?}}{T} - H \text{?} \frac{P \text{?}}{T \text{?}} ) \qquad ( 16 )$$

$$\text{?} \text{indicates text missing or illegible when filed}$$

where M represents the molar mass of water (18×10-3 kg mol-1), R is the universal gas constant (8.314 J mol-1 K-1), Pv, sat w and Pv, sat e are the pressures of water vapor at the surface temperature (T) and at the environment temperature (Te), respectively, HR is the relative humidity (45%), and km is the convective mass transfer coefficient (0.01 m s⁻¹).

The variation of thermal conductivity (keff), pore size (Rc), and vaporization enthalpy (hevap) as the function of porosity (E) were obtained by fitting the experimental data (FIG. 34B):

$${k}_{eff} = 0.532 + 2.834 \times {10}^{- 4} ( e \text{?} + e \text{?} ) \text{?} {Wm}^{- 2} {K}^{- 1} \text{?} \qquad ( 17 )$$

$${R}_{c} = - 579.249 + 588.425 ( 1 - e \text{?} ) \text{?} μm \text{?} \qquad ( 18 )$$

$${h}_{evap} = 1027.432 + 8.875 ( e \text{?} + e \text{?} ) \text{?} {Jg}^{- 1} \text{?} \qquad ( 19 )$$

$$\text{?} \text{indicates text missing or illegible when filed}$$

To simulate the salt distribution in NHSEs, a mass flux of NaCl (Jevap) was applied on the desalination interface:[119]

$${J}_{evap} = \frac{{q}_{evap} \text{?}}{{h}_{evap} ρ} \qquad ( 20 )$$

$$ρ ( c , T ) = {ρ}_{0} ( T ) \to β \cdot c \qquad ( 21 )$$

$$\text{?} \text{indicates text missing or illegible when filed}$$

where qevap represents the evaporation heat flux on the surface, c is the brine concentration, hevap is the latent heat at the desalination interface, ρ is the brine density, which is affected by the brine concentration and temperature, and β (0.033 kg mol⁻¹) is a proportionality constant. The salt concentration at the bottom of NHSEs was set as the 3.5%-20%.

Thus, this embodiment of the present invention relates to composite nanofibrous hydrogels for solar desalination applicable in high-salinity brine. The efficient mass transfer, low thermal conductivity, and reduced vaporization enthalpy inherited from the hybrid nanofiber network are crucial for the efficient and stable performance in solar desalination. The mechanistic insights regarding the microstructural, thermodynamic, and kinetic processes and their quantitative contributions to the evaporation performance are conducive to the engineering of other HSEs. Developing mesoscopic models, such as those involving lattice Boltzmann method, would further advance the understanding of mass and heat transfer in the porous media.[116] In most cases, promoting hydration of the polymeric network by introducing appropriate functional groups would be helpful to lower vaporization enthalpy with minimal impact on the water transfer. Finally, device-level designs for solar evaporators would provide additional performance enhancement with improved efficiency in solar energy utilization. Kirigami Enabled 3D Evaporator Arrays

### Kirigami 3D Evaporator Array

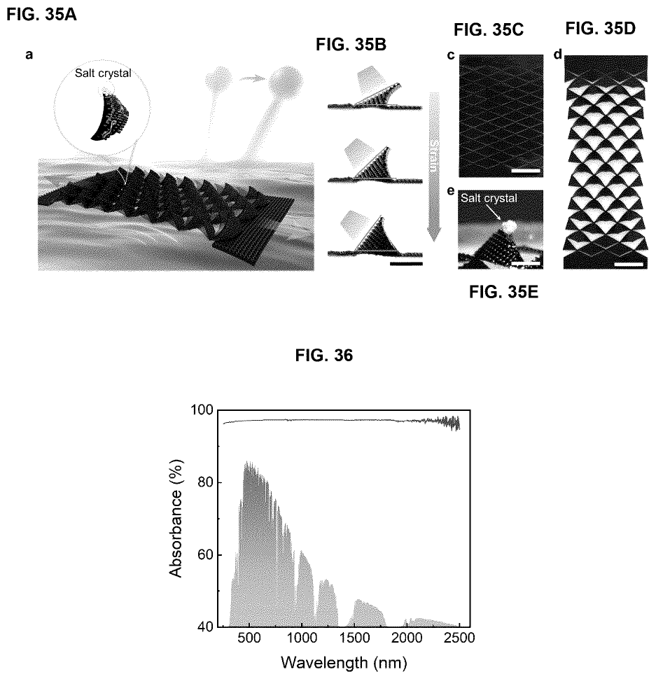

The dynamic 3D interfacial solar evaporators (ISE) using a kirigami-based hydrogel solar evaporator (KHSE) of the present invention achieves scalable fabrication, durable desalination even in saturated brine and dynamic solar tracking. FIG. 35A In this scheme, a mechanically robust hydrogel membrane based on aramid nanofibers (ANF) is utilized as the substrate that enables engineering of kirigami without structural failure. The periodic triangular notches on the substrate generate 3D conical arrays under strain, leading to scalable 3D evaporators. The 3D conical array decorated with biomimetic microstructures realizes localized crystallization at peak, achieving stable evaporation of 3.4 kg m-2 h⁻¹ in saturated brine. Additionally, high reconfigurability of KHSE allows for dynamic solar trackable evaporation by varying imposed strain, enhancing the energy efficiency in field applications. FIG. 35B Using the solar trackable KHSE, the water production increased by 18% and 82% compared to static KHSE and conventional flat evaporator, respectively. The proposed kirigami-enabled production of scalable and solar-trackable 3D evaporators provide inspiration for the next generation of ISEs.

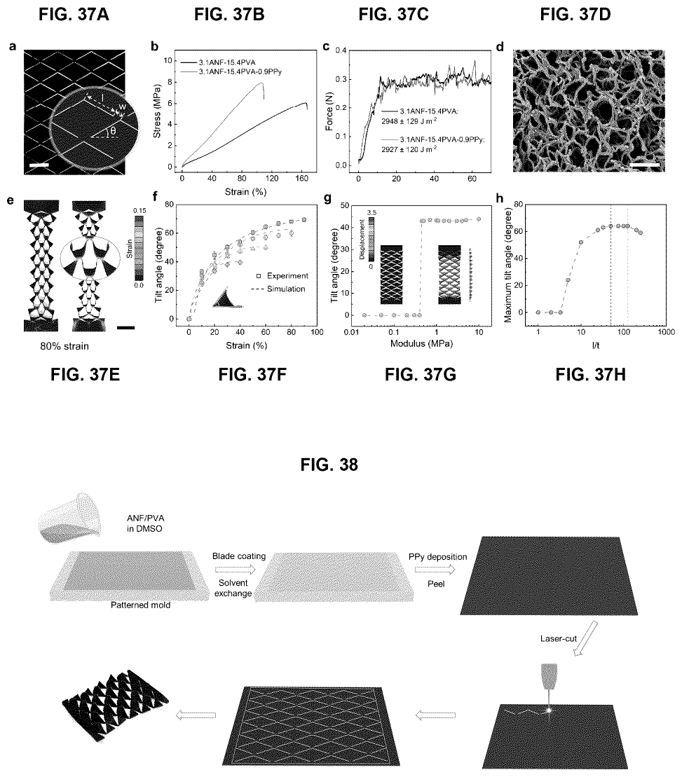

A dynamic 3D interfacial solar evaporator (ISE) using KHSE technology according to the present invention achieves scalable fabrication, durable desalination even in saturated brine, and dynamic solar tracking. Fabrication of KHSE begins with a hydrogel membrane assembled by an aramid nanofibers (ANF) network incorporating polyvinyl alcohol (PVA) via solution-based processing. After coating polypyrrole (PPy) for photothermal conversion (FIG. 36), the resultant hydrogel membrane was laser engraved with periodic triangular notches to obtain KHSE (FIGS. 37A and 38). The ANF-PVA hydrogel offers an ideal engineering platform for kirigami due to its high mechanical robustness. The pristine ANF-PVA hydrogel membrane with the thickness of 220 μm exhibits a high stretchability ≈120%, strength ≈8 MPa, and modulus ≈10 MPa (FIG. 37B). A high fracture energy up to 2948 J m-2 is achieved (FIG. 37C), which is comparable to some silicon rubbers. 32 Excellent mechanical properties of ANF-PVA hydrogel inherit from the interconnected ANF network toughened by PVA via extensive hydrogen bonds (FIG. 37D).[150]The loaded PPy is arranged along ANFs via π-π stacking and hydrogen bonding, providing efficient solar absorption without compromising the mechanical properties.[151] These advantages bless kirigami membrane with structure robustness under tension. For example, a KHSE membrane (1=1 cm, dimensionless ratio 1/w=10, and θ=20°) can withstand 80% of strain without fracture (FIG. 37E). Finite element analysis (FEA) exhibits the strain distribution at the joint, which is well below the failure strain of the substrate, indicating its high stretchability.

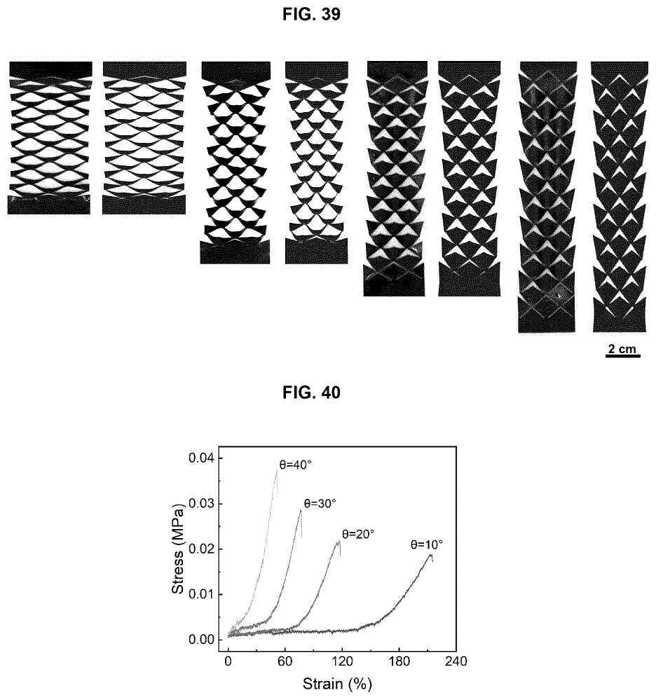

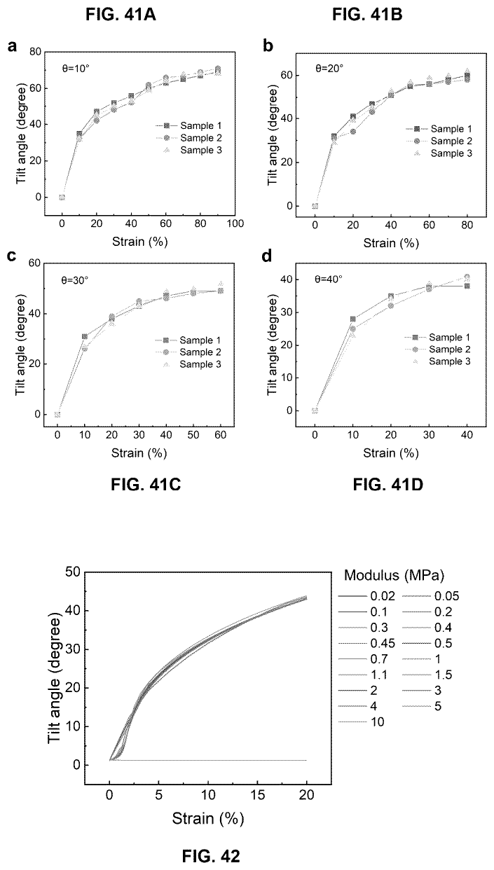

Deformability is an essential consideration of KHSE, as it determines the formation of 3D evaporators and the dynamic behavior for solar tracking. In this design, both the stretchability and tilt angle (γ) are tunable by varying cut angles (FIG. 37F and FIGS. 39-41). The KHSE with 10° of cut angle demonstrates the highest stretchability of 200% and the largest saturated tilt angle of 70° under 90% strain. The maximum deformation is inversely proportional to the size of cut angle, partly due to the increased structure stiffness (S), which depends on the elastic modulus (E), cut angle (θ), cut length (l), and thickness of the membrane (t) via a scaling law:

$$S ∝ E sin θ {lt}^{3} \qquad ( 1 )$$

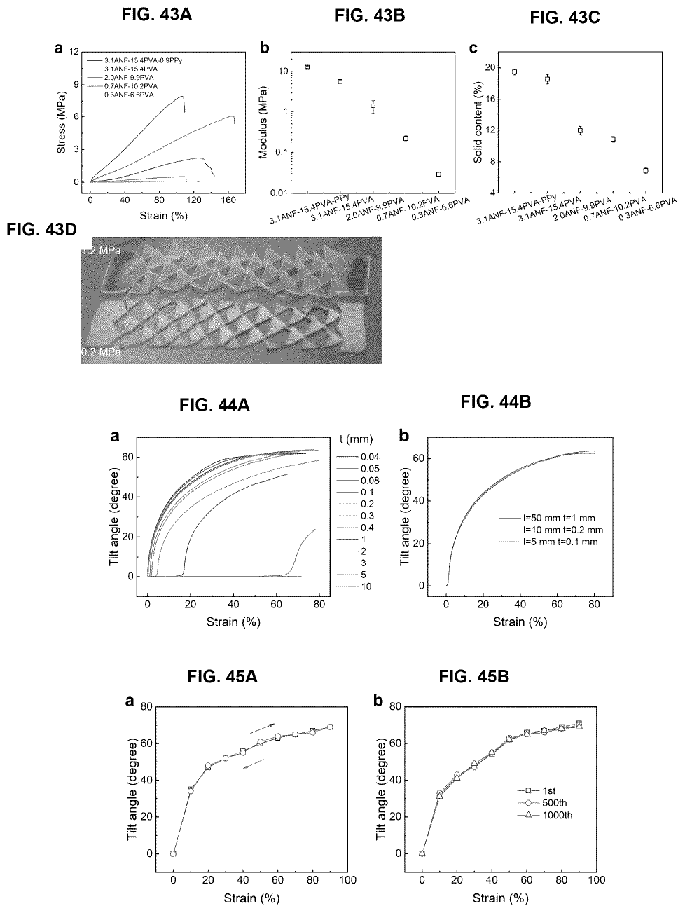

Indeed, high structural stiffness will resist the out-of-plane deformation. However, for soft hydrogels, enough modulus is necessary to overcome the gravity of swollen hydrogel for popping up. The deforming behavior of KHSE was simulated with different modulus under a gravitational field. The out-of-plane deformation occurs when the modulus increases to 0.45 MPa for KHSE (1=10 mm, t=0.2 mm, and 0=20°), indicating the modulus threshold for popping up, which is consistent with the experimental observation (FIG. 37G and FIGS. 42-43). Though a modulus of 0.45 MPa is sufficient for out-of-plane deformation, stiffer hydrogel membrane facilitates sturdy construction of KHSE, preventing overall bending during operation. In this regard, high modulus of ANF-PVA-PPy hydrogel makes it advantageous for base material of KHSE. The ratio of cut length and thickness (l/t) also affects the range of tilt angle by influencing the stiffness (FIG. 37H and FIG. 44A). With the same cut length of 10 mm, the decreased thickness of membrane results in the larger tilt angle, followed by a plateau that indicates the range for optimum design. The subsequent decline of maximum tilt angle might be ascribed to the decreased structural stiffness that cannot bear the buckling. This finding improves the designing flexibility of KHSE by increasing the thickness within a certain range (l/t=50˜125) for higher stiffness, without deteriorating its deformation characteristics. These design principles are applicable across various scales (FIG. 44B), providing universal guidelines for designing KHSE or other hydrogel-based kirigami devices. Additionally, the KHSE demonstrates stable deformation over 1000 cycles of 80% elongation (FIG. 45), indicating its durability for prolonged utilization.

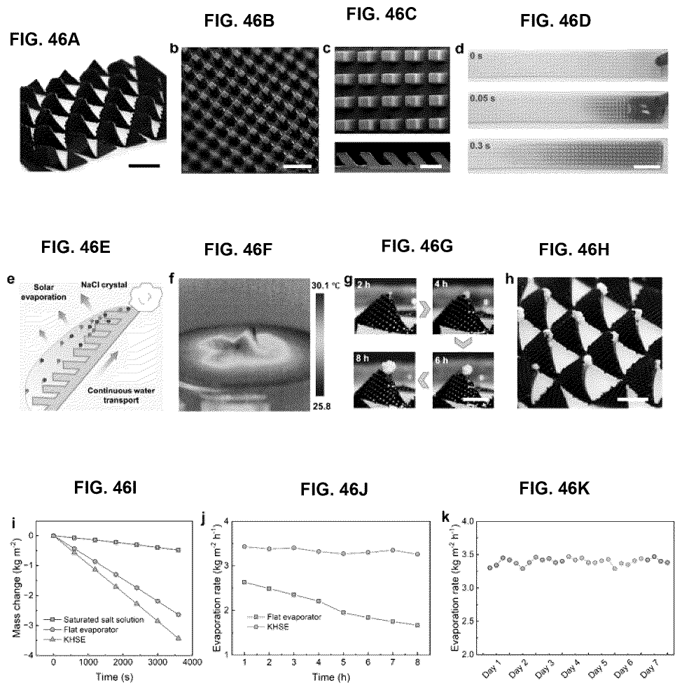

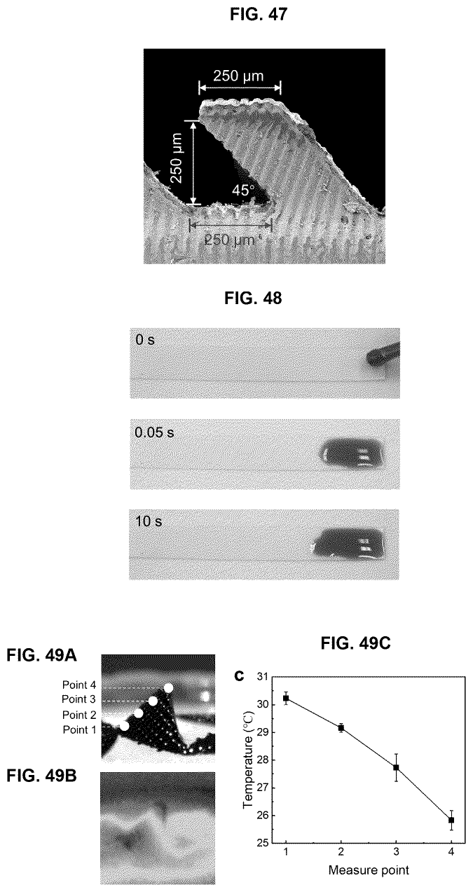

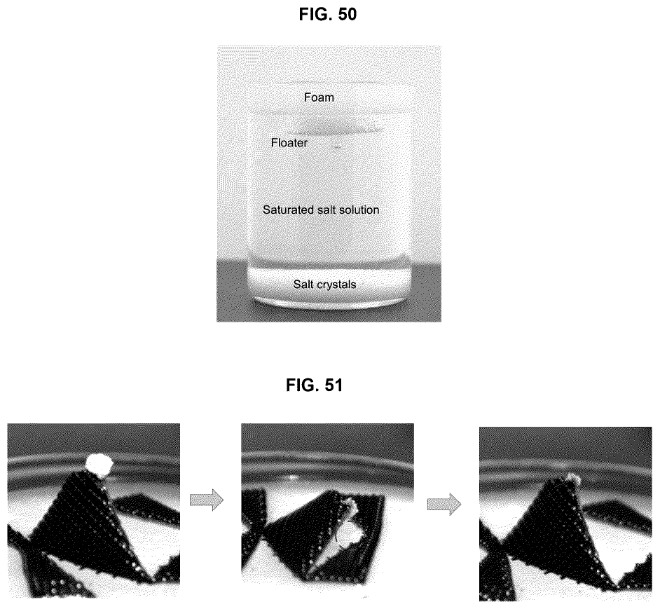

Efficient water supply is vital for solar desalination in brine, especially for KHSE with distant evaporating interface from the water surface and limited contact area with water. The solution-based processing enables generating micropatterns via a simple molding method (FIG. 38). Here, biomimetic 3D capillary ratchets were patterned on KHSE to facilitate the liquid suction. [152] The anti-swelling ANF-PVA hydrogel allows for maintaining the precise microstructure in the fully hydrated state (FIG. 46B). SEM images exhibit the periodic arrangement of micro-ratchets on the surface, with the same width, height, and pitch of 250 m, and tilt angle of 45° (FIG. 46C and FIG. 47). These micropatterns enable water to fully spread on a hydrogel strip (0.5 cm×7 cm) in 0.3 s (FIG. 46D), showing the greater water transfer capacity than that of smooth hydrogel (FIG. 48). 3D conical arrays generated by stretching the KHSE, incorporating the enhanced water transfer, achieved stable solar desalination in saturated brine via localized crystallization. This peak-preferential crystallization can be attributed to the variation of evaporation efficiency along the 3D evaporator (FIG. 46E). With the same energy input to the evaporator, lower temperature indicates more energy consumption for evaporation, leading to the higher evaporation rate.[124] As the most efficient evaporation at the peak, the liquid here is prior to reach the critical crystallization concentration, resulting in the peak-preferential crystallization. Both the KHSE unit and arrays demonstrates the capability of localized crystallization when purifying saturated brine (FIG. 46G, FIG. 46H & FIG. 50). Furthermore, the reconfigurability of KHSE enables salt crystals to drop from the evaporator by repeated stretch (FIG. 51), providing a novel approach to salt cleanup without manual intervention.

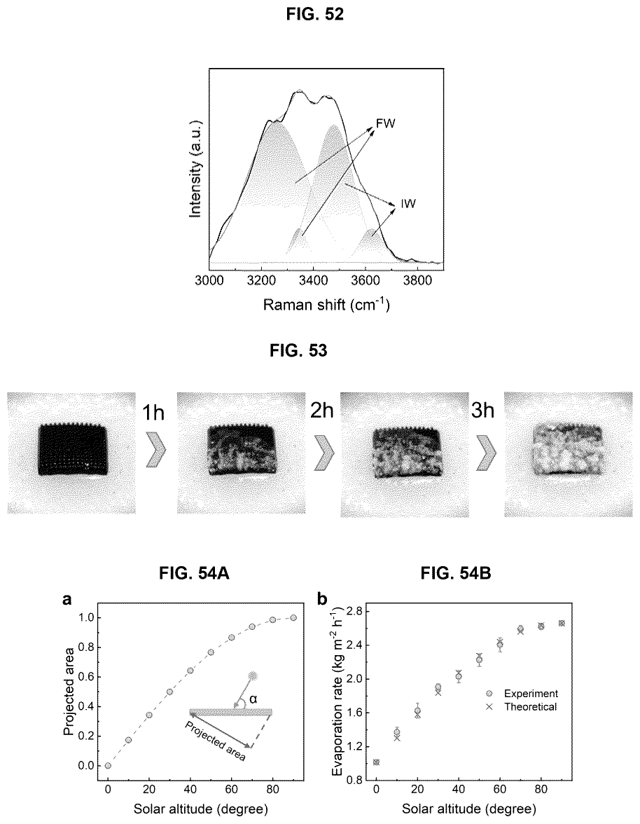

The KHSE (tilt angle of 40°) demonstrates a high evaporation rate of 3.4 kg m⁻²h⁻¹, 1.3 and 7 times higher than that of flat evaporator and pure solution, respectively (FIG. 46I). Five factors may contribute to the efficient evaporation. First, the tilt evaporator positions the evaporating interface away from the water surface, significantly reducing the heat loss to the bulk water, resulting in effective heat localization. Second, the out-of-plane deformation allows for additional evaporation at the backside, increasing the effective evaporative area. Third, the temperature gradient induces the Marangoni effect, driving the liquid from the high temperature to the low temperature under surface tension gradient.[132] Redistribution of energy by liquid migration also facilitates the energy efficiency of KHSE. Fourth, the hydrated polymeric networks in KHSE reduce vaporization enthalpy by activating water molecules, which is validated by characterizing water states via Raman spectrum (FIG. 52). [124] Finally, the top of KHSE demonstrates lower temperature than the ambient environment, which can capture energy from the surroundings under the law of thermodynamics, in favor of higher energy efficiency.[128] Besides efficient evaporation, the KHSE also demonstrates more stable evaporation than that of flat evaporator in 8 h of continuous evaporation (FIG. 46J and FIG. 52). Stable evaporation of KHSE in saturated bine for 7 days (8 h per day) indicates it durability for long-term solar desalination (FIG. 46K).

### Solar Tracking

Generally, evaporation performance is evaluated in the laboratory with static and constant solar illumination, which is divergent from the real-world application. [135] Actually, the deviation of the evaporator interface from the incident angle of sunlight causes degraded evaporation performance.[147] The ultimate evaporation value consists of two components, natural evaporation, and solar-induced evaporation. The latter significantly reduces with the decline of the projected area, which represents the energy gain from solar, and scales with the sine of solar altitude (FIG. 54A). Assuming solar is the sole energy source to the system, the evaporation rate (E) can be described as the following equation:

$$E = {E}_{n} + sin α \cdot {E}_{0} \qquad ( 2 )$$

where E_n, E_0, and α represent the natural evaporation, the solar-induced evaporation with vertically incident sunlight, and the solar altitude, respectively, which is consistent with experimental observations (FIG. 54B). The measured E_0 is 1.64 kg m-2 h-1, which accounts for 62% of the total evaporation, indicating the significance of stable projected area.

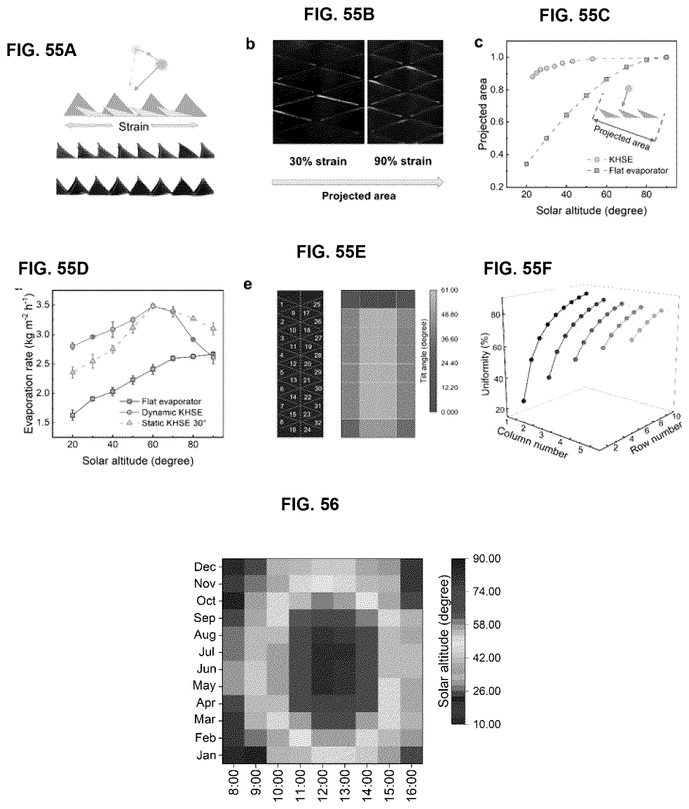

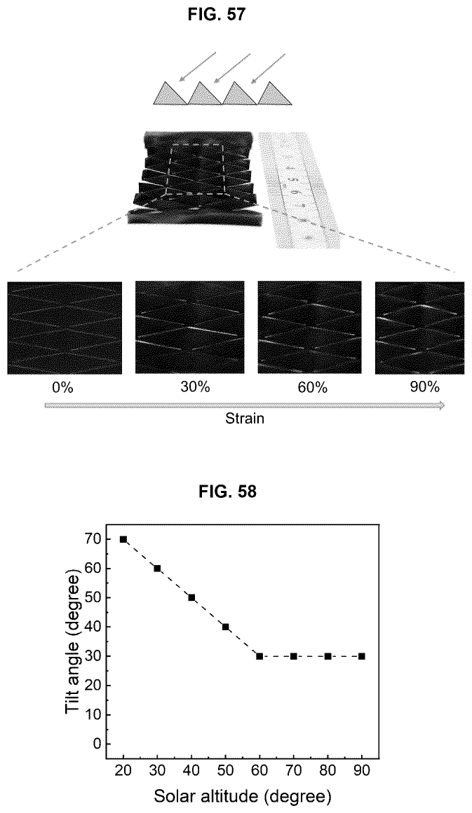

The reconfigurability of KHSE allows for dynamically capturing solar trajectory by aligning the tilt angle to the solar altitude (FIG. 55A). Considering the 8 h of effective evaporation in a day (for instance, Hong Kong), the KHSE with the maximum tilt angle of 700 is sufficient for solar tracking (FIG. 56). On the other hand, longitudinal stretching of the KHSE results in the transverse structural shrinkage, which decreases the projected area (FIG. 35 and FIG. 57). Therefore, the KHSE with the cut angle of 10° was utilized for solar tracking due to its large span of tilt angle and low Poisson's ratio. The KHSE maintains 88% of projected area with the solar altitude of 20°, 158% higher than that of flat evaporator (FIG. 55C). However, solar tracking throughout does not necessarily result in the maximum evaporation. The dynamic KHSE that tracks solar throughout demonstrates low tilt angle as the solar altitude exceeds 60°, which is insufficient to fully exploit the advantages of 3D evaporator (FIG. 55D). This limitation results in a lower evaporation rate despite the large projected area. Inversely, the static KHSE with the tilt angle of 30° demonstrates more efficient evaporation compared to dynamic KHSE at this stage, followed by decreased instantly below that of dynamic KHSE. This difference indicates the compromise between energy efficiency and energy gain of KHSE, which determines the ultimate evaporation rate. As the optimal configuration, the tilt angle of KHSE should vary with solar movement until the solar altitude is up to 60° and maintain at 30° (FIG. 58).

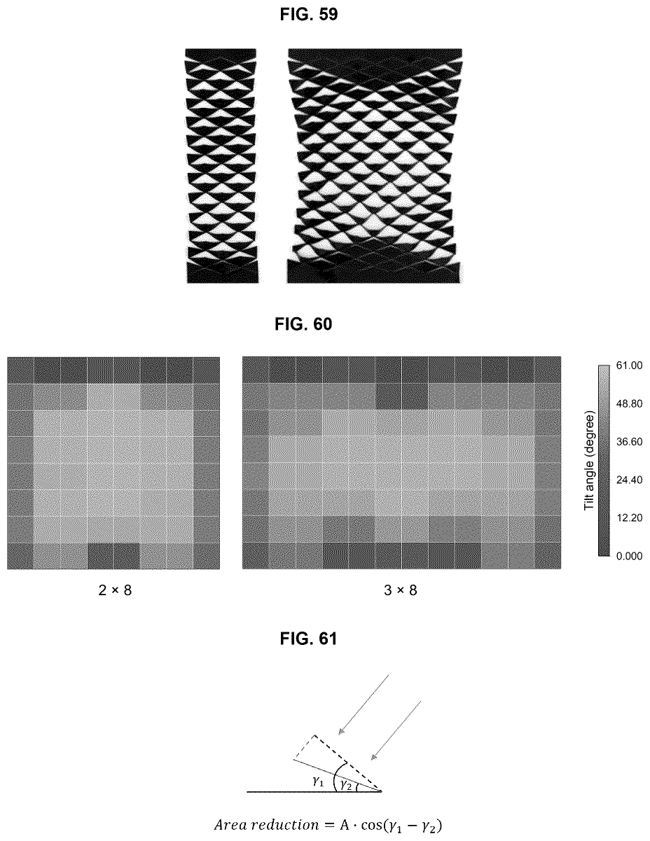

However, for large-scale KHSE, the units at both ends of KHSE are difficult to fully pop up, resulting in heterogeneous solar tracking at different areas (FIG. 59). The variation of tilt angle in KHSE via FEA simulation was systematically investigated. The KHSE was gridded into 4Nr Nc parts, where Nr and Nc represent number of rows and number of columns, respectively (FIG. 55E). Statistics of tilt angle in KHSE were visualized as color maps to indicate the variation (FIG. 55E and FIG. 60). The deviation of projected area scales with the cosine of the differential seat angle (FIG. 61). When a deviation within 10% is defined in the projected area as acceptable for uniform deformation, the difference between tilt angles should be less than 25°. The uniformity (P) can be summarized as (FIG. 55F):

$$P = ( 1 - \frac{4 {N}_{c} - 1}{2 {N}_{r} {N}_{c}} ) \qquad ( 3 )$$

which provides guidelines for designing large-scale KHSEs.

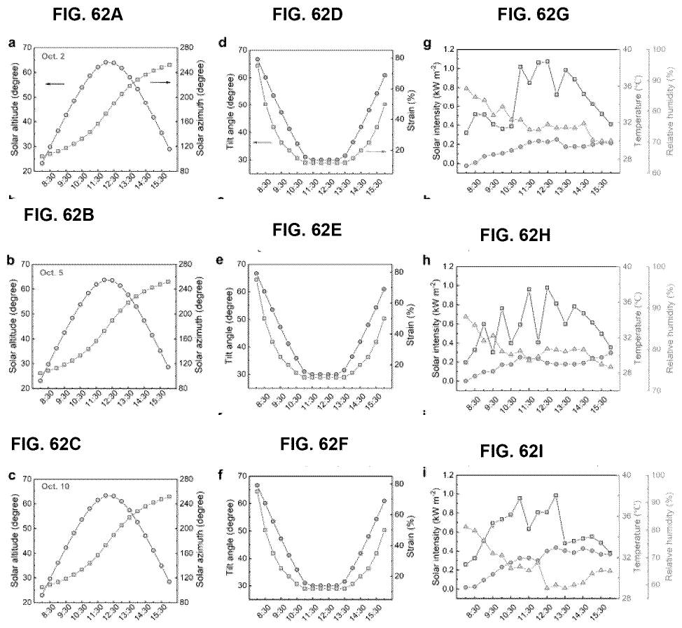

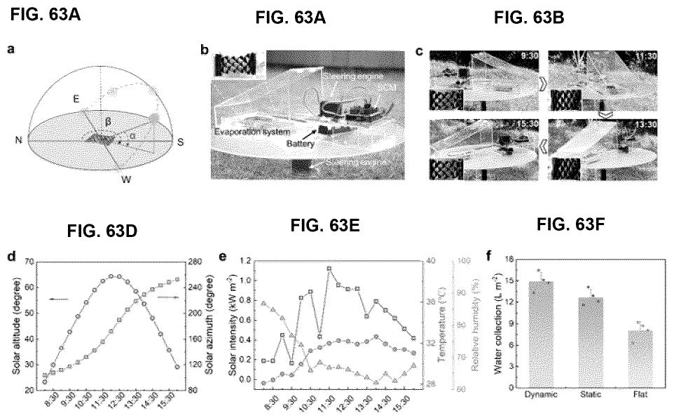

Finally, the evaporation performance of KHSE is evaluated under the real-world conditions. The movement of sun causes changes in both altitude and azimuth (FIG. 63A), representing variations in the angle of sunlight in vertical and horizontal directions, respectively. A prototype was developed to facilitate the angle coupling of KHSE with solar (FIG. 63B). In this design, two steering engines (DSSERVO, DS3115) are integrated to control the stretch of KHSE and rotation of the evaporation system, which accommodates solar altitude and azimuth, respectively. A single-chip microcontroller (SCM, Arduino Uno) was used to program the rotation of the steering engines to match the solar trajectory, which is predictable based on location and time. [152] All components are powered by a battery, which can also be replaced with solar panels to provide a low carbon emission system. The outdoor tests were conducted on sunny days from 8 am to 16 pm with the automatic solar tracking system (FIG. 63C). At noon the solar angles, solar intensity, temperature, and humidity changes were also recorded (FIG. 63D, FIG. 63E and FIG. 62). An efficient water yield of 14.9 L m-2 was achieved by KHSE with solar tracking, 18% and 85% higher than that of static KHSE and flat evaporator with the same scale (FIG. 63F). The simple structure incorporated efficient water production, which indicates the promising prospects for KHSE in practical applications.

The present invention presents a strategy for the scalable construction of solar-trackable 3D evaporators based on a kirigami-design. Excellent mechanical properties including high strength, toughness, and modulus combined with hydrated polymeric network of ANF-PVA hydrogel membranes make it favorable for designing kirigami evaporators. The hydrogel-based evaporator offers a versatile platform that allows for applying various exiting technologies to optimize the performance, for example, reducing vaporization enthalpy and optimizing water supply. Further integrating a horizontal rotation component into the large-scale KHSE to capture the solar azimuth will promote its widespread implementation. Finally, systematic investigations of KHSE reveals intrinsic connections between mechanics of base materials and deforming behaviors, which may also provide insights for engineering other kirigami enabled devices based on hydrogels.

In fabrication of KHSE, preparation of ANF and PVA dispersions in dimethyl sulfoxide (DMSO) are followed by a method reported elsewhere. [150, 153] Fabrication of ANF-PVA hydrogel membrane begins with mixing 3 wt % ANF dispersion and 15% PVA dispersion for a homogenous mixture. Then ANF-PVA membrane was obtained by blade-coating the mixture with controlled thickness followed by fully solvent exchange with deionized water. The hydrogel membrane with micropatterns was produced by blade-coating the mixture on a mold. Whereafter, the obtained hydrogel membrane was immersed into 0.1 M FeCl₃ solution and then exposed to pyrrole vapor to obtain ANF-PVA-PPy membrane. The periodic triangular notches were laser patterned on the hydrogel membrane to obtain KHSE.

The microstructures of KHSE were characterized using SEM (Hitachi S4800 FEG) after drying by critical point dryer (CPD; Tousimis Autosamdri 931). The solar adsorption was obtained using UV-vis-NIR spectrometer (UV 36001 plus, Shimadzu). Raman spectra (Horiba Scientific LabRAM HR Evolution) was used to characterize water states in KHSE.

Mechanical performance of hydrogel membranes was evaluated on a tensile-compressive tester. The samples with the size of 30 mm×5 mm×0.22 mm were loaded with the stretching rate of 1% s-1. Tearing test was carried out to evaluate the fracture energy (Γ) of hydrogel membranes (20 mm×50 mm×0.22 mm). Γ was calculated by Γ=2F/t, where F and t represent steady state tearing force and thickness of the membrane, respectively.

Tests of the solar evaporation of KHSE were conducted under a solar simulator (Aulight, CEL-S500-T5) with the intensity of 1 kW m-2. The incident solar angle was regulated by rotating the light outlet. The mass loss and temperature of KHSE was recorded using an electronic balance (Mettler Toledo) and an infrared camera (Fluke Ti480), respectively. The temperature and humidity during the evaporation tests were 25° C. and 50%, respectively. Projected area of KHSE was estimated by capturing photos perpendicular to the tilt angle and measuring the area (FIG. 56). For outdoor test, the solar intensity was measured using a solar power meter (Tenmars, TM-207). Ambient temperature and humidity were recorded using a multidetector (Renke, COS-03). The solar altitude and azimuth were collected from Hong Kong Observatory.

Numerical simulations were conducted with a commercial FEA package ABAQUS 2019 (SIMULIA). The Poisson's ratio was 0.27 for all simulations, which was obtained by experiment. Four-node shell elements (Abaqus element type S4R with a mesh seed size of 0.1 mm) with reduced integration and hourglass control were used to discretize the KHSE with a width much smaller than the length/width. Gravity was loaded on the whole model with an acceleration of 9.80 m/s2, where the influence of gravity on post-buckling behaviours was concerned. The density of KHSEs was maintained as 1.03 g/cm3. The tilt angle (γ) was calculated in a 3D rectangular coordinates system:

$$γ = {tan}^{- 1} ( \frac{{z}_{1} - {z}_{2}}{{y}_{1} - {y}_{2}} ) \qquad ( 4 )$$

where [symbol] (y) [symbol] _1,z [symbol] _1) and [symbol] (y) [symbol] _2,z [symbol] _2) represent the coordinates of the vertex and the joint in a tilt cut, respectively. Deforming uniformity of KHSE was evaluated by statistically analysing all tilt angles as demonstrated in FIGS. 55E and 55F.

In summary, the present invention is an efficient assembly of conducting polymer nanostructures templated by ANFs for the construction of electronically conductive hydrogels. The hybrid polymeric nanofiber network combines high electrical conductivity with desired mechanical robustness and manufacturability for device applications. The versatile CNHs with tunable compositions and properties may expand the materials toolbox for the design of hydrogel bioelectronics, which currently focuses on quite limited options based on PEDOT [34]. The processing of CNHs does not involve high temperatures or dramatic volume changes as do those required for the reported PEDOT hydrogels [2,25]. This feature makes CNHs favorable for further integration with bioactive molecules, microfluidic channels or other functional components for advanced bio-interfaces. The good biocompatibility of CNHs also permits development of implantable system for neural and cardiac applications. On the other hand, tissue engineering technologies may benefit from CNHs due to their excellent electrical properties along with biomimetic nanofibrous structures. Further tuning of mechanical behaviors, structural anisotropy, mesoscale patterning or other attributes of CNHs may create diverse opportunities for controlling behaviors of cells and tissues through electromechanically coupled interfaces.

The above are only specific implementations of the invention and are not intended to limit the scope of protection of the invention. Any modifications or substitutes apparent to those skilled in the art shall fall within the scope of protection of the invention. Therefore, the protected scope of the invention shall be subject to the scope of protection of the claims.

### REFERENCES

The cited references in this application are incorporated herein by reference in their entirety and are as follows:
[1] Liu, Y. et al. Soft and elastic hydrogel-based microelectronics for localized low-voltage neuromodulation. Nat. Biomed. Eng. 3, 58-68 (2019).
[2] Yuk, H. et al. 3D printing of conducting polymers. Nat. Commun. 11, 1604 (2020).
[3] Keplinger, C. et al. Stretchable, Transparent, Ionic Conductors. Science 341, 984-987 (2013).
[4] Li, T. et al. Fast-moving soft electronic fish. Sci. Adv. 3, e1602045 (2017).
[5] Dvir, T. et al. Nanowired three-dimensional cardiac patches. Nat. Nanotechnol. 6, 720-725 (2011).
[6] Park, J. et al. Electrically Conductive Hydrogel Nerve Guidance Conduits for Peripheral Nerve Regeneration. Adv. Funct. Mater. 30, 2003759 (2020).
[7] Zhou, X., Zhao, F., Guo, Y., Rosenberger, B. & Yu, G. Architecting highly hydratable polymer networks to tune the water state for solar water purification. Sci. Adv. 5, eaaw5484 (2019).
[8] Zhao, F. et al. Highly efficient solar vapour generation via hierarchically nanostructured gels. Nat. Nanotechnol. 13, 489-495 (2018).
[9] Kim, C. C., Lee, H. H., Oh, K. H. & Sun, J. Y. Highly stretchable, transparent ionic touch panel. Science 353, 682-7 (2016).
[10] Li, L., Zhang, Y., Lu, H., Wang, Y. & Liu, T. Cryopolymerization enables anisotropic polyaniline hybrid hydrogels with superelasticity and highly deformation-tolerant electrochemical energy storage. Nat. Commun. 11, 62 (2020).
[11] Mo, F. et al. A Self-Healing Crease-Free Supramolecular All-Polymer Supercapacitor. Adv. Sci. 8, 2100072 (2021).
[12] Wang, Y. K. et al. A Highly Elastic and Reversibly Stretchable All-Polymer Supercapacitor. Angew. Chem. Int. Ed. 58, 15707-15711 (2019).
[13] Zhang, W., Feng, P., Chen, J., Sun, Z. & Zhao, B. Electrically conductive hydrogels for flexible energy storage systems. Prog. Polym. Sci. 88, 220-240 (2019).
[14] Yang, C. & Suo, Z. Hydrogel ionotronics. Nat. Rev. Mater. 3, 125-142 (2018).
[15] Li, Y. & Gregory, S. Diffusion of ions in sea water and in deep-sea sediments. Geochim. Cosmochim. Acta 38, 703-714 (1974).
[16] Southern, E. M. Detection of specific sequences among DNA fragments separated by gel electrophoresis. J. Mol. Biol. 98, 503-517 (1975).
[17] Ahn, Y., Lee, H., Lee, D. & Lee, Y. Highly conductive and flexible silver nanowire-based microelectrodes on biocompatible hydrogel. ACS Appl. Mater. Interfaces 6, 18401-18407 (2014).
[18] Han, L. et al. Mussel-Inspired Adhesive and Conductive Hydrogel with Long-Lasting Moisture and Extreme Temperature Tolerance. Adv. Funct. Mater. 28, (2018).
[19] Liang, Y., Zhao, X., Hu, T., Han, Y. & Guo, B. Mussel-inspired, antibacterial, conductive, antioxidant, injectable composite hydrogel wound dressing to promote the regeneration of infected skin. J. Colloid Interface Sci. 556, 514-528 (2019).
[20] Pan, L. et al. Hierarchical nanostructured conducting polymer hydrogel with high electrochemical activity. PNAS 109, 9287-9292 (2012).
[21] Kim, Y. et al. Stretchable nanoparticle conductors with self-organized conductive pathways. Nature 500, 59-63 (2013).
[22] White, S. I. et al. Electrical Percolation Behavior in Silver Nanowire-Polystyrene Composites: Simulation and Experiment. Adv. Funct. Mater. 20, 2709-2716 (2010).
[23] Ohm, Y. et al. An electrically conductive silver-polyacrylamide-alginate hydrogel composite for soft electronics. Nat. Electron. 4, 185-192 (2021).
[24] Zhao, Y. et al. Hierarchically Structured Stretchable Conductive Hydrogels for High-Performance Wearable Strain Sensors and Supercapacitors. Matter 3, 1196-1210 (2020).
[25] Lu, B. et al. Pure PEDOT:PSS hydrogels. Nat. Commun. 10, 1043 (2019).
[26] Li, G. et al. Highly Conducting and Stretchable Double-Network Hydrogel for Soft Bioelectronics. Adv. Mater. 34, 2200261 (2022).
[27] Feig, V. R., Tran, H., Lee, M. & Bao, Z. Mechanically tunable conductive interpenetrating network hydrogels that mimic the elastic moduli of biological tissue. Nat. Commun. 9, 2740 (2018).
[28] Zhu, J. et al. Branched Aramid Nanofibers. Angew. Chem. Int. Ed. 56, 11744-11748 (2017).
[29] Xu, L., Zhao, X., Xu, C. & Kotov, N. A. Water-Rich Biomimetic Composites with Abiotic Self-Organizing Nanofiber Network. Adv. Mater. (2018).
[30] He, H. et al. Ultrastrong and multifunctional aerogels with hyperconnective network of composite polymeric nanofibers. Nat. Commun. 13, 4242 (2022).
[31] Chakraborty, P. et al. A Self-Healing, All-Organic, Conducting, Composite Peptide Hydrogel as Pressure Sensor and Electrogenic Cell Soft Substrate. ACS Nano 13, 163-175 (2019).
[32] Shevach, M., Fleischer, S., Shapira, A. & Dvir, T. Gold Nanoparticle-Decellularized Matrix Hybrids for Cardiac Tissue Engineering. Nano Lett. 14, 5792-5796 (2014).
[33] Kim, S. et al. High-performance, polymer-based direct cellular interfaces for electrical stimulation and recording. NPG Asia Mater. 10, 255-265 (2018).
[34] Yuk, H., Lu, B. & Zhao, X. Hydrogel bioelectronics. Chem. Soc. Rev. 48, 1642-1667 (2019).
[35] Lee, S. et al. In vivo transduction of ETV2 improves cardiac function and induces vascular regeneration following myocardial infarction. Exp. Mol. Med. 51, 1-14 (2019).
[36] Park, B. W. et al. In vivo priming of human mesenchymal stem cells with hepatocyte growth factor-engineered mesenchymal stem cells promotes therapeutic potential for cardiac repair. Sci. Adv. 6, eaay6994 (2020).
[37] Pu, X. et al. Ultrastretchable, transparent triboelectric nanogenerator as electronic skin for biomechanical energy harvesting and tactile sensing. Sci. Adv. 3, e1700015 (2017).
[38] Ma, L. et al. Super-stretchable zinc-air batteries based on an alkaline-tolerant dual-network hydrogel electrolyte. Adv. Energy Mater. 9, 1803046 (2019).
[39] Lai, J. et al. Neuromorphic sensorimotor loop embodied by monolithically integrated, low-voltage, soft e-skin. Science (80-.). 742, 735-742 (2023).
[40] Liu, Y. et al. Soft and elastic hydrogel-based microelectronics for localized low-voltage neuromodulation. Nat. Biomed. Eng. 3, 58-68 (2019).
[41] Walker, B. W. et al. Rational design of microfabricated electroconductive hydrogels for biomedical applications. Prog. Polym. Sci. 92, 135-157 (2019).
[42] Park, J. et al. Electrically conductive hydrogel nerve guidance conduits for peripheral nerve regeneration. Adv. Funct. Mater. 30, 2003759 (2020).
[43] Yao, B. et al. Ultrahigh-Conductivity Polymer Hydrogels with Arbitrary Structures. Adv. Mater. 29, (2017).
[44] Fang, Y. et al. Micelle-enabled self-assembly of porous and monolithic carbon membranes for bioelectronic interfaces. Nat. Nanotechnol. 16, 206-213 (2021).
[45] Zhou, T. et al. 3D Printable High Performance Conducting Polymer Hydrogel for All-Hydrogel Bioelectronics. Nat. Mater. 2022.01.29.478311 (2023) doi:10.1038/s41563-023-01569-2.
[46] Wang, L. et al. Functionalized helical fibre bundles of carbon nanotubes as electrochemical sensors for long-term in vivo monitoring of multiple disease biomarkers. Nat. Biomed. Eng. 4, 159-171 (2020).
[47] Guo, S. et al. Integrated contact lens sensor system based on multifunctional ultrathin MoS2 transistors. Matter 4, 969-985 (2021).
[48] Yuk, H., Lu, B. Y. & Zhao, X. H. Hydrogel bioelectronics. Chem. Soc. Rev. 48, 1642-1667 (2019).
[49] Hua, M. et al. Tough-Hydrogel Reinforced Low-Tortuosity Conductive Networks for Stretchable and High-Performance Supercapacitors. Adv. Mater. 33, 1-8 (2021).
[50] He, H. et al. Hybrid assembly of polymeric nanofiber network for robust and electronically conductive hydrogels. Nat. Commun. 14, 759 (2023).
[51] Cogan, S. F. Neural stimulation and recording electrodes. Annu. Rev. Biomed. Eng. 10, 275-309 (2008).
[52] Johnson, E. R. et al. Revealing Noncovalent Interactions. J. Am. Chem. Soc. 132, 6498-6506 (2010).
[53] Ajmal, C. M., Bae, S. & Baik, S. A Superior Method for Constructing Electrical Percolation Network of Nanocomposite Fibers: In Situ Thermally Reduced Silver Nanoparticles. Small 15, 1803255 (2019).
[54] Chun, K. Y. et al. Highly conductive, printable and stretchable composite films of carbon nanotubes and silver. Nat. Nanotechnol. 5, 853-857 (2010).
[55] Hur, J. et al. Polypyrrole/Agarose-Based Electronically Conductive and Reversibly Restorable Hydrogel. ACS Nano 8, 10066-10076 (2014).
[56] Mo, F. et al. A Self-Healing Crease-Free Supramolecular All-Polymer Supercapacitor. Adv. Sci. 8, 2100072 (2021).
[57] Choi, C. et al. Improvement of system capacitance via weavable superelastic biscrolled yarn supercapacitors. Nat. Commun. 7, 1-8 (2016).
[58] Yao, B. et al. Hydrogel Ionotronics with Ultra-Low Impedance and High Signal Fidelity across Broad Frequency and Temperature Ranges. Adv. Funct. Mater. 32, 1-10 (2022).
[59] Pang, A. L., Arsad, A. & Ahmadipour, M. Synthesis and factor affecting on the conductivity of polypyrrole: a short review. Polym. Adv. Technol. 32, 1428-1454 (2021).
[60] Huang, H., Zeng, X., Li, W. & Wang, H. Reinforced conducting hydrogels prepared from the in situ polymerization of aniline in an aqueous solution of sodium alginate. J. Mater. Chem. A Mater. energy Sustain. 2, 16516-16522 (2014).
[61] Cheng, Y., Ren, X., Duan, L. & Gao, G. A transparent and adhesive carboxymethyl cellulose/polypyrrole hydrogel electrode for flexible supercapacitors. J. Mater. Chem. C 8, 8234-8242 (2020).
[62] Trexler, M. M. et al. Synthesis and mechanical properties of para-aramid nanofibers. J. Polym. Sci. Part B Polym. Phys. 57, 563-573 (2019).
[63] Xu, L., Zhao, X., Xu, C. & Kotov, N. A. Water-Rich Biomimetic Composites with Abiotic Self-Organizing Nanofiber Network. Adv. Mater. (2018).
[64] Ni, J. et al. Strong fatigue-resistant nanofibrous hydrogels inspired by lobster underbelly. Matter 4, 1919-1934 (2021).
[65] He, H. et al. Ultrastrong and multifunctional aerogels with hyperconnective network of composite polymeric nanofibers. Nat. Commun. 13, 4242 (2022).
[66] Jiang, Y. et al. Topological supramolecular network enabled high-conductivity, stretchable organic bioelectronics. Science (80-.). 375, 1411-1417 (2022).
[67] Park, M. et al. Highly stretchable electric circuits from a composite material of silver nanoparticles and elastomeric fibres. Nat. Nanotechnol. 7, 803-809 (2012).
[68] He, S. et al. Stretchable supercapacitor based on a cellular structure. J. Mater. Chem. A 4, 10124-10129 (2016).
[69] Wu, S., Moody, K., Kollipara, A. & Zhu, Y. Highly Sensitive, Stretchable, and Robust Strain Sensor Based on Crack Propagation and Opening. ACS Appl. Mater. Interfaces 15, 1798-1807 (2023).
[70] Li, Y., Poon, C. C. Y. & Zhang, Y. T. Analog integrated circuits design for processing physiological signals. IEEE Rev. Biomed. Eng. 3, 93-105 (2010).
[71] Tandon, N. et al. Electrical stimulation systems for cardiac tissue engineering. Nat. Protoc. 4, 155-173 (2009).
[72] Chakraborty, P. et al. A Self-Healing, All-Organic, Conducting, Composite Peptide Hydrogel as Pressure Sensor and Electrogenic Cell Soft Substrate. ACS Nano 13, 163-175 (2019).
[73] Zhang, F. et al. Gelatin-based hydrogels combined with electrical stimulation to modulate neonatal rat cardiomyocyte beating and promote maturation. Bio-Design Manuf. 4, 100-110 (2021).
[74] Yoshida, S., Sumomozawa, K., Nagamine, K. & Nishizawa, M. Hydrogel Microchambers Integrated with Organic Electrodes for Efficient Electrical Stimulation of Human iPSC-Derived Cardiomyocytes. Macromol. Biosci. 19, 1-9 (2019).
[75] Stoller, M. D. & Ruoff, R. S. Best practice methods for determining an electrode material's performance for ultracapacitors. Energy Environ. Sci. 3, 1294-1301 (2010).
[76] L. Zhou, Y. Tan, J. Wang, W. Xu, Y. Yuan, W. Cai, S. Zhu, J. Zhu, Nat. Photonics 2016, 10, 393.
[77] E. Chiavazzo, M. Morciano, F. Viglino, M. Fasano, P. Asinari, Nat. Sustain. 2018, 1, 763.
[78] P. Tao, G. Ni, C. Song, W. Shang, J. Wu, J. Zhu, G. Chen, T. Deng, Nat. Energy 2018, 3, 1031.
[79] L. Wu, Z. Dong, Z. Cai, T. Ganapathy, N. X. Fang, C. Li, C. Yu, Y. Zhang, Y. Song, Nat. Commun. 2020, 11, 521.
[80] Y. Guo, H. Lu, F. Zhao, X. Zhou, W. Shi, G. Yu, Adv. Mater. 2020, 32, 1907061.
[81] X. Chen, S. He, M. M. Falinski, Y. Wang, T. Li, S. Zheng, D. Sun, J. Dai, Y. Bian, X. Zhu, J. Jiang, L. Hu, Z. J. Ren, Energy Environ. Sci. 2021, 14, 5347.
[82] N. Li, K. Shao, J. He, S. Wang, S. Li, X. Wu, J. Li, C. Guo, L. Yu, P. Murto, J. Chen, X. Xu, Small 2023, 19, 2301474.
[83] N. Li, L. Qiao, J. He, S. Wang, L. Yu, P. Murto, X. Li, X. Xu, Adv. Funct. Mater. 2021, 31, 2008681.
[84] F. Li, N. Li, S. Wang, L. Qiao, L. Yu, P. Murto, X. Xu, Adv. Funct. Mater. 2021, 31, 2104464.
[85] X. Zhou, F. Zhao, Y. Guo, Y. Zhang, G. Yu, Energy Environ. Sci. 2018, 11, 1985.
[86] Y. Guo, G. Yu, Acc. Mater. Res. 2021, 2, 374.
[87] F. Zhao, X. Zhou, Y. Shi, X. Qian, M. Alexander, X. Zhao, S. Mendez, R. Yang, L. Qu, G. Yu, Nat. Nanotechnol. 2018, 13, 489.
[88] X. Zhou, F. Zhao, Y. Guo, B. Rosenberger, G. Yu, Sci. Adv. 2019, 5, eaaw5484.
[89] J. Zeng, Q. Wang, Y. Shi, P. Liu, R. Chen, Adv. Energy Mater. 2019, 9, 1900552.
[90] M. Tan, J. Wang, W. Song, J. Fang, X. Zhang, J. Mater. Chem. A 2019, 7, 1244.
[91] Y. Kuang, C. Chen, S. He, E. M. Hitz, Y. Wang, W. Gan, R. Mi, L. Hu, Adv. Mater. 2019, 31, 1900498.
[92] W. Zhao, H. Gong, Y. Song, B. Li, N. Xu, X. Min, G. Liu, B. Zhu, L. Zhou, X.-X. Zhang, J. Zhu, Adv. Funct. Mater. 2021, 31, 2100025.
[93] L. Li, N. He, B. Jiang, K. Yu, Q. Zhang, H. Zhang, D. Tang, Y. Song, Adv. Funct. Mater. 2021, 31, 2104380.
[94] W. Xu, X. Hu, S. Zhuang, Y. Wang, X. Li, L. Zhou, S. Zhu, J. Zhu, Adv. Energy Mater. 2018, 8, 1702884.
[95] Z. Wang, J. Gao, J. Zhou, J. Gong, L. Shang, H. Ye, F. He, S. Peng, Z. Lin, Y. Li, F. Caruso, Adv. Mater. 2023, 35, 2209015.
[96] N. He, Y. Yang, H. Wang, F. Li, B. Jiang, D. Tang, L. Li, Adv. Mater. 2023, 35, 2300189.
[97] C. Ma, Q. Liu, Q. Peng, G. Yang, M. Jiang, L. Zong, J. Zhang, ACS Nano 2021, 15, 19877.
[98] W. Tu, Z. Wang, Q. Wu, H. Huang, Y. Liu, M. Shao, B. Yao, Z. Kang, J. Mater. Chem. A 2020, 8, 10260.
[99] H. Yang, Y. Sun, M. Peng, M. Cai, B. Zhao, D. Li, Z. Liang, L. Jiang, ACS Nano 2022, 16, 2511.
[100] H. Zou, X. Meng, X. Zhao, J. Qiu, Adv. Mater. 2023, 35, 2207262.
[101] X. Dong, Y. Si, C. Chen, B. Ding, H. Deng, ACS Nano 2021, 15, 12256.
[102] X. Dong, L. Cao, Y. Si, B. Ding, H. Deng, Adv. Mater. 2020, 32, 1908269.
[103] X. Liu, F. Chen, Y. Li, H. Jiang, D. D. Mishra, F. Yu, Z. Chen, C. Hu, Y. Chen, L. Qu, Adv. Mater. 2022, 34, 2203137.
[104] Y. Guo, L. S. de Vasconcelos, N. Manohar, J. Geng, K. P. Johnston, G. Yu, Angew. Chem., Int. Ed. 2022, 61, e202114074.
[105] J. Jiang, H. Jiang, Y. Xu, L. Ai, Desalination 2022, 539, 115943.
[106] T. Xu, Y. Xu, J. Wang, H. Lu, W. Liu, J. Wang, Chem. Eng. J. 2021, 415, 128893.
[107] J. Li, L. Yan, X. Li, W. Song, Y. Li, J. Environ. Chem. Eng. 2022, 10, 107690.
[108] X. Chen, Z. Wu, D. Lai, M. Zheng, L. Xu, J. Huo, Z. Chen, B. Yuan, M.-L. Fu, J. Mater. Chem. A 2020, 8, 22645.
[109] P. Liu, Y.-b. Hu, X.-Y. Li, L. Xu, C. Chen, B. Yuan, M.-L. Fu, Angew. Chem., Int. Ed. 2022, 61, e202208587.
[110] X. Zhou, Y. Guo, F. Zhao, W. Shi, G. Yu, Adv. Mater. 2020, 32, 2007012.
[111] L. Xu, X. Zhao, C. Xu, N. A. Kotov, Adv. Mater. 2018, 30, 1703343.
[112] H. He, H. Li, A. Pu, W. Li, K. Ban, L. Xu, Nat. Commun. 2023, 14, 759.
[113] H. Liu, H. Li, Z. Wang, X. Wei, H. Zhu, M. Sun, Y. Lin, L. Xu, Adv. Mater. 2022, 34, 2207350.
[114] B. Luo, J. Wen, H. Wang, S. Zheng, R. Liao, W. Chen, O. Mahian, X. Li, Energy Environ. Mater. 2023, 6, e12353.
[115] Y. Alsaid, S. Wu, D. Wu, Y. Du, L. Shi, R. Khodambashi, R. Rico, M. Hua, Y. Yan, Y. Zhao, D. Aukes, X. He, Adv. Mater. 2021, 33, 2008235.
[116] S. Chen, G. D. Doolen, Annu. Rev. Fluid Mech. 1998, 30, 329.
[117] H. Wang, R. Zhang, D. Yuan, S. Xu, L. Wang, Adv. Funct. Mater. 2020, 30, 2003995.
[118] R. Fillet, V. Nicolas, V. Fierro, A. Celzard, Int. J. Heat Mass Transfer 2021, 181, 121852.
[119] L. Zhang, X. Li, Y. Zhong, A. Leroy, Z. Xu, L. Zhao, E. N. Wang, Nat. Commun. 2022, 13, 849.
[120] Tao P, et al. Solar-driven interfacial evaporation. Nat. Energy 3, 1031-1041 (2018).
[121] Wang Z, et al. Pathways and challenges for efficient solar-thermal desalination. Sci. Adv. 5, eaax0763.
[122] Wang Z, Wu X, He F, Peng S, Li Y. Confinement Capillarity of Thin Coating for Boosting Solar-Driven Water Evaporation. Adv. Funct. Mater. 31, 2011114 (2021).
[123] Liang H, et al. Thermal Efficiency of Solar Steam Generation Approaching 100% through Capillary Water Transport. Angew. Chem. Int. Ed. 58, 19041-19046 (2019).
[124] Ghasemi H, et al. Solar steam generation by heat localization. Nat. Commun. 5, 4449 (2014).
[125] Liu H, et al. Bioinspired Self-Standing, Self-Floating 3D Solar Evaporators Breaking the Trade-Off between Salt Cycle and Heat Localization for Continuous Seawater Desalination. Adv. Mater. 35, 2301596 (2023).
[126] Zhao F, et al. Highly efficient solar vapour generation via hierarchically nanostructured gels. Nat. Nanotechnol. 13, 489-495 (2018).
[127] Zhou X, Zhao F, Guo Y, Zhang Y, Yu G. A hydrogel-based antifouling solar evaporator for highly efficient water desalination. Energy Environ. Sci. 11, 1985-1992 (2018).
[128] Wu X, Wu Z, Wang Y, Gao T, Li Q, Xu H. All-Cold Evaporation under One Sun with Zero Energy Loss by Using a Heatsink Inspired Solar Evaporator. Adv. Sci. 8, 2002501 (2021).
[129] Li X, et al. Enhancement of Interfacial Solar Vapor Generation by Environmental Energy. Joule 2, 1331-1338 (2018).
[130] Shi Y, et al. A 3D Photothermal Structure toward Improved Energy Efficiency in Solar Steam Generation. Joule 2, 1171-1186 (2018).
[131] Zou H, Meng X, Zhao X, Qiu J. Hofmeister Effect-Enhanced Hydration Chemistry of Hydrogel for High-Efficiency Solar-Driven Interfacial Desalination. Adv. Mater. 35, 2207262 (2023).
[132] Guo Y, Zhao X, Zhao F, Jiao Z, Zhou X, Yu G. Tailoring surface wetting states for ultrafast solar-driven water evaporation. Energy Environ. Sci. 13, 2087-2095 (2020).
[133] Chen C, Kuang Y, Hu L. Challenges and Opportunities for Solar Evaporation. Joule 3, 683-718 (2019).
[134] Wu L, et al. Highly efficient three-dimensional solar evaporator for high salinity desalination by localized crystallization. Nat. Commun. 11, 521 (2020).
[135] Li L, et al. Highly Salt-Resistant 3D Hydrogel Evaporator for Continuous Solar Desalination via Localized Crystallization. Adv. Funct. Mater. 31, 2104380 (2021).
[136] Xia Y, et al. Spatially isolating salt crystallisation from water evaporation for continuous solar steam generation and salt harvesting. Energy Environ. Sci. 12, 1840-1847 (2019).
[137] Shan H, Ye Z, Yu J, Wang R, Xu Z. Improving solar water harvesting via airflow restructuring using 3D vapor generator. Device, 100065 (2023).
[138] Singh SC, et al. Solar-trackable super-wicking black metal panel for photothermal water sanitation. Nat. Sustain. 3, 938-946 (2020).
[139] Bian Y, et al. Enhanced Contactless Salt-Collecting Solar Desalination. ACS Appl. Mater. Interfaces 14, 34151-34158 (2022).
[140] Xu L, Shyu T C, Kotov N A. Origami and Kirigami Nanocomposites. ACS Nano 11, 7587-7599 (2017).
[141] Rafsanjani A, Jin L, Deng B, Bertoldi K. Propagation of pop ups in kirigami shells. Proc. Natl. Acad. Sci. U.S.A. 116, 8200-8205 (2019).
[142] Rafsanjani A, Zhang Y, Liu B, Rubinstein S M, Bertoldi K. Kirigami skins make a simple soft actuator crawl. Sci. Robot. 3, eaar7555 (2018).
[143] Liu H, et al. Robust and Multifunctional Kirigami Electronics with a Tough and Permeable Aramid Nanofiber Framework. Adv. Mater. 34, 2207350 (2022).
[144] Rao Z, et al. Curvy, shape-adaptive imagers based on printed optoelectronic pixels with a kirigami design. Nat. Electron. 4, 513-521 (2021).
[145] Yang P-K, et al. Paper-Based Origami Triboelectric Nanogenerators and Self-Powered Pressure Sensors. ACS Nano 9, 901-907 (2015).
[146] Babaee S, Pajovic S, Rafsanjani A, Shi Y, Bertoldi K, Traverso G. Bioinspired kirigami metasurfaces as assistive shoe grips. Nat. Biomed. Eng. 4, 778-786 (2020).
[147] Li J, et al. Aerodynamics-assisted, efficient and scalable kirigami fog collectors. Nat. Commun. 12, 5484 (2021).
[148] Xu S, et al. Assembly of micro/nanomaterials into complex, three-dimensional architectures by compressive buckling. Science 347, 154-159 (2015).
[149] Lamoureux A, Lee K, Shlian M, Forrest S R, Shtein M. Dynamic kirigami structures for integrated solar tracking. Nat. Commun. 6, 8092 (2015).
[150] Li Y, et al. Printed Kirigami Organic Photovoltaics for Efficient Solar Tracking. Adv. Funct. Mater. 32, 2204004 (2022).
[151] Wang Z, Xiang C, Yao X, Le Floch P, Mendez J, Suo Z. Stretchable materials of high toughness and low hysteresis. Proc. Natl. Acad. Sci. U.S.A. 116, 5967-5972 (2019).
[152] Xu L, Zhao X, Xu C, Kotov N A. Water-Rich Biomimetic Composites with Abiotic Self-Organizing Nanofiber Network. Adv. Mater. 30, 1703343 (2018).
[153] He H, Li H, Pu A, Li W, Ban K, Xu L. Hybrid assembly of polymeric nanofiber network for robust and electronically conductive hydrogels. Nat. Commun. 14, 759 (2023).
[154] Feng S, et al. Three-dimensional capillary ratchet-induced liquid directional steering. Science 373, 1344-1348 (2021).
[155] Hoyt D V. A model for the calculation of solar global insolation. Sol. Energy 21, 27-35 (1978).
[156] Zhu J, et al. Branched Aramid Nanofibers. Angew. Chem. Int. Ed. 56, 11744-11748 (2017).
[157] Vassetti, D., Pagliai, M. & Procacci, P. Assessment of GAFF2 and OPLS-AA General Force Fields in Combination with the Water Models TIP3P, SPCE, and OPC3 for the Solvation Free Energy of Druglike Organic Molecules. J. Chem. Theory Comput. 15, 1983-1995 (2019).
[158] Nian, B., Xu, Y.-J. & Liu, Y. Molecular dynamics simulation for mechanism revelation of the safety and nutrition of lipids and derivatives in food: State of the art. Food Res. Int. 145, 110399 (2021).
[159] Han, L. et al. Transparent, Adhesive, and Conductive Hydrogel for Soft Bioelectronics Based on Light-Transmitting Polydopamine-Doped Polypyrrole Nanofibrils. Chem. Mater. 30, 5561-5572 (2018).
[160] Lu, T. & Chen, F. Multiwfn: A multifunctional wavefunction analyzer. J. Comput. Chem. 33, 580-592 (2012).
[161] Liu, J. et al. Hydrogen-bonding regulated supramolecular chirality with controllable biostability. Nano Res. 15, 2226-2234 (2022).
[162] Valdés-Tresanco, M. S., Valdés-Tresanco, M. E., Valiente, P. A. & Moreno, E. Gmx_MMPBSA: A New Tool to Perform End-State Free Energy Calculations with GROMACS. J. Chem. Theory Comput. 17, 6281-6291 (2021).
[163] Johnson, E. R. et al. Revealing Noncovalent Interactions. J. Am. Chem. Soc. 132, 6498-6506(2010).
[164] Meng, Y. et al. All-graphene core-sheath microfibers for all-solid-state, stretchable fibriform supercapacitors and wearable electronic textiles. Adv. Mater. 25, 2326-2331 (2013).
[165] Zhu, Y. et al. Carbon-Based Supercapacitors Produced by Activation of Graphene. Science (80-.). 332, 1537-1542 (2011).
[166] He, S. et al. Stretchable supercapacitor based on a cellular structure. J. Mater. Chem. A 4, 10124-10129 (2016).
[167] Choi, C. et al. Improvement of system capacitance via weavable superelastic biscrolled yarn supercapacitors. Nat. Commun. 7, 1-8 (2016).
[168] Li, J., Cao, Z., Hu, H. & Ho, D. Diameter-optimized PVA@PPy nanofibers: MXene interlayer space expansion without sacrificing electron transport. J. Mater. Chem. C 13056-13063 (2022) doi:10.1039/d2tc00231k.
[169] Huang, H. et al. Facile fabrication of MnO2-embedded 3-D porous polyaniline composite hydrogel for supercapacitor electrode with high loading. High Perform. Polym. 32, 286-295 (2020).
[170] Zhou, H., Yao, W., Li, G., Wang, J. & Lu, Y. Graphene/poly(3,4-ethylenedioxythiophene) hydrogel with excellent mechanical performance and high conductivity. Carbon N. Y. 59, 495-502 (2013).
[171] Antiohos, D. et al. Compositional effects of PEDOT-PSS/single walled carbon nanotube films on supercapacitor device performance. J. Mater. Chem. 21, 15987-15994 (2011).
[172] Yao, B. et al. Ultrahigh-Conductivity Polymer Hydrogels with Arbitrary Structures. Adv. Mater. 29, (2017).
[173] Cheng, Y., Ren, X., Duan, L. & Gao, G. A transparent and adhesive carboxymethyl cellulose/polypyrrole hydrogel electrode for flexible supercapacitors. J. Mater. Chem. C 8, 8234-8242 (2020).
[174] Chen, C. R., Qin, H., Cong, H. P. & Yu, S. H. A Highly Stretchable and Real-Time Healable Supercapacitor. Adv. Mater. 31, 1-10 (2019).

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. An electroconductive hydrogel formed by a hybrid assembly of polymeric nanofiber networks that self-organize into a template of highly connected 3D nanostructures on to which conducting polymers assemble through in-situ synthesis.

2. The hydrogel of claim 1 wherein the highly connected 3D nanostructures are formed from aramid nanofibers (ANFs) and polypyrrole (PPy) forms the conducting polymers resulting in an ANF-PPy hydrogel.

3. The hydrogel of claim 1 wherein the highly connected 3D nanostructures are formed from aramid nanofibers (ANFs), polyvinyl alcohol (PVA) welds the fibrillar joints and polypyrrole (PPy) forms the conducting polymers resulting in an ANF-PVA-PPy hydrogel.

4. The hydrogel of claim 1 wherein the highly connected 3D nanostructures are formed from aramid nanofibers (ANFs) and the conducting polymers are poly(3,4-ethylenedioxythiophene) (PEDOT) resulting in an ANF-PEDOT hydrogel.

5. The hydrogel of claim 1 wherein the highly connected 3D nanostructures are formed from cellulose and polypyrrole (PPy) forms the conducting polymers resulting in cellulose-PPy hydrogel.

6. The hydrogel of claim 1 wherein the resultant hybrid network of conductive nanofiber hydrogels (DNHs) exhibit a combination of high electronic conductivity of ˜8,000 S m⁻¹, structural robustness of 1.6 MPa to 17.6 MPa, preferrably ˜9.4 MPa, and stretchability of 55% to 20%, preferably ˜34% to ˜37%, without sacrificing porosity or water content.

7. The hydrogel of claim 6 wherein the water content is ˜80%.

8. A method for forming an electroconductive hydrogel comprising the steps of:
dispersing aramid nanofibers (ANFs) in dimethyl sulfoxide (DMSO);
conducting a solvent exchange with water to generate hydrogels with connective 3D fibrillar networks that serve as templates for the assembly of conducting polymers;
incorporating polyvinyl alcohol (PVA) during the processing of the hydrogels to weld the fibrillar joints via hydrogen bonding;
infiltrating monomers into the nanoporous hydrogels in an aqueous media; and
polymerizing the hydrogels with added oxidants.

9. The method of claim 8 wherein the monomers are pyrrole (Py) and the oxidants are FeCl₃.

10. The method of claim 9 wherein the synthesisation is based on 1.9% ANF, 9.5% PVA matrix and 0.3 wt % pyrrole, polymerized under pH 7 and at a temperature 0° C. for 2 hours.

11. A method of forming a bioelectrode pattern comprising the steps of:
masking ANF-PVA hydrogel samples with waterproof adhesive tapes;
treating the masked hydrogel samples with Py and FeCl₃ solutions; and
incorporating PPy into the ANF-PVA matrix only in the area exposed by the mask, leading to custom patterns of CNH.

12. A method for measuring bioelectric activities of humans with the bioelectrode pattern of claim 11 comprising the steps of laminating the bioelectrode patterns onto the skin of the human.

13. The method for measuring bioelectric activities of humans according to claim 12 wherein an activity is an electromyogram (EMG).

14. The method for measuring bioelectric activities of humans according to claim 11 wherein the activity is an electrocardiogram (ECG).

15. A method for preparing conductive ANF-PVA hydrogels comprising the steps of:
dissolving Kevlar para-aramid pulp and PVA in dimethylsulfoxide (DMSO) under magnetic stirring at 95° C. for 7 days;
dissolving Kevlar para-aramid pulp and PVA in dimethylsulfoxide (DMSO) under magnetic stirring at 95° C. for 7 days;
mixing the resulting ANF and PVA liquids;
pouring the mixture into a mould or casting the mixture on a flat steel plate using a film coater; and
solidifying the ANF-PVA mixture through solvent exchange in deionized (DI) water for 24 hours.

16. The method of claim 15 further including the steps of:
pre-soaking ANF-PVA hydrogel in a pyrrole solution under ice-water bath with vibration at 160 rpm by a shaker;
adding FeCl₃ after 1 h of pre-soaking into the solution;
allowing polymerization to proceed for 2 h to form samples; and
soaking the samples in 0.5 mM FeCl₃ solution to form ANF-PVA-PPy.

17. A method for patterning ANF-PPy as a custom-built bioelectrode, comprising the steps of:
preparing a PDMS mold with microchannels by microfabrication techniques;
placing the PDMS mold face down on a water-soluble tape,
infusing an ANF dispersion into the microchannels;
generating a patterned ANF hydrogel by soaking the PDMS in water for both the removal of the tape and solidification of the ANF dispersion; and
embedding a final ANF-PPy pattern within the PDMS channel through the incorporation of PPy into the hydrogel network by sequential treatment with Py and FeCl₃ solutions.

18. A nanofibrous hydrogel solar evaporators (NHSE) comprising an electroconductive hydrogel according to claim 1 having an intrinsic open network with high porosity, wherein the hydrogel is made by dispersing ANF and PVA in DMSO followed by mixing in the liquid phase and PPy is added as a photothermal conversion material to polymerize along the nanofibers.

19. The NHSE of claim 18 with a tunable open porous network to achieve high-performance solar desalination, whereby the tuning of porosity was achieved by varying the ANF and PVA contents in the hydrogels.

20. The NHSE of claim 18 wherein the hydrogel had an ANF between 0.7% and 1.0% and a PVA between 4.7% and 7.4% PVA.

21. A method of producing ANF-PVA hydrogels for NHSE comprising the steps of:
separately dissolving Kevlar para-aramid pulp and PVA in dimethyl sulfoxide (DMSO) under magnetic stirring at 95° C. for 7 days, respectively;
mixing the ANF and PVA dispersions a ratio of 1:1;
pouring the mixture into a mold;
allowing an exchange of DMSO with deionized water;
controlling the solid content of the hydrogel by diluting the ANF/PVA mixture with DMSO or evaporating excess DMSO in vacuum oven;
wherein the PVA hydrogels are prepared using a freeze-thaw method in which PVA aqueous solution is poured into a mold and then frozen at −24° C.;
thereafter the frozen mixture is thawed at room temperature and repeating the PVA freeze-thaw cycles at least five times.

22. The method according to claim 21 wherein the PVA has Mn≈75.000; hydrolysis degree of 96%≈98%; and the ANF is included at 1.5% and the PVA is included at 10%.

23. The method of claim 22 further including the step of:
coating the structure with PPy by soaking the hydrogels with FeCl₃ solution;
exposing the soaked hydrogels to pyrrole vapor at 4° C. for 10 min for polymerization; and
washing the hydrogels with DI water to remove unreacted ions and pyrrole.

24. A 3D interfacial solar evaporators (ISE) using a kirigami-based hydrogel solar evaporator (KHSE) comprising:
a substrate formed from a mechanically robust hydrogel membrane based on aramid nanofibers (ANF);
periodic triangular notches on the substrate to form 3D conical arrays under strain; and
biomimetic microstructures on the conical arrays to realize localized crystallization at their peak.

25. The KHSE of claim 24 which is highly reconfigurability to allow for dynamic solar trackable evaporation by varying imposed strain, thereby enhancing the energy efficiency in field applications.

26. The KHSE of claim 25 further including two steering engines attached to the KHSE to control the stretch of the KHSE to cause it to change in one or both of altitude and azimuth; and a processor programmed to cause the steering engines to move the KHSE to match solar trajectory.

27. The KHSE of claim 24 in which both its stretchability and tilt angle (γ) are tunable by varying the cut angles, wherein the maximum deformation is inversely proportional to the size of the cut angle, partly due to the increased structure stiffness (S), which depends on the elastic modulus (E), cut angle (θ), cut length (l), and thickness of the membrane (t) via a scaling law.

28. The KHSE of claim 25 wherein the cut angle is up to about 10°, the highest stretchability is about 200% and the largest saturated tilt angle is about 70° under 90% strain.

29. The KHSE of claim 24 further including biomimetic 3D capillary ratchets patterned on the conical arrays of the KHSE to facilitate liquid suction.

30. A method of forming a 3D interfacial solar evaporator using a kirigami-based hydrogel solar evaporator (KHSE) comprising the steps of:
forming a hydrogel membrane assembled with an aramid nanofiber (ANF) network incorporating polyvinyl alcohol (PVA) via solution-based processing;
coating the network with polypyrrole (PPy) for photothermal conversion to achieve a resultant hydrogel membrane; and
laser engraving the membrane with periodic triangular notches to form 3D conical arrays of the KHSE.
